(19) United States
(12) Patent Application Publication
(10) Pub. No.: US 2026/0356879 A1
(43) Pub. Date: Nov. 12, 2026

(54) CELASTROL DERIVATIVES

(71) Applicant: Celloram Inc., Cleveland, OH (US)

(72) Inventors: Seunghwan LIM, Cleveland, OH (US); Tej Kumar PAREEK, Cleveland, OH (US); Liraz LEVI, Cleveland, OH (US); Elizabeth MEYERS, Cleveland, OH (US); William J. GREENLEE, Cleveland, OH (US); Seong-Jin KIM, Cleveland, OH (US)

(73) Assignee: Celloram Inc., Cleveland, OH (US)

(21) Appl. No.: 18/994,317

(22) PCT Filed: Jul. 12, 2023
(86) PCT No.: PCT/US2023/027456

§ 371 Date: Jan. 14, 2025

Related U.S. Application Data
(60) Provisional application No. 63/389,593, filed on Jul. 15, 2022.

(51) Int. Cl. C07J 63/00; A61K 31/58; A61P 29/00

(52) U.S. Cl. CPC C07J 63/008; A61K 31/58; A61P 29/00

(57) ABSTRACT

The present disclosure relates to compounds that are derivatives of the compound, celastrol, and pharmaceutical compositions that include these celastrol derivative compounds. The disclosure also relates to uses of the celastrol derivative compounds described herein, including uses in medicaments, therapy, and methods of treating celastrol-responsive disease or disorders, such as cancers, inflammatory and/or autoimmune disorders, neurological disorders, brain injuries and disorders, obesity-related diseases and disorders, and/or liver-related diseases and disorders.

### CROSS REFERENCE TO RELATED APPLICATIONS

This application claims priority of U.S. Provisional Patent Application No. 63/389,593, filed Jul. 15, 2022, the entirety of which is hereby incorporated by reference herein.

### FIELD

The present disclosure relates to compounds that are derivatives of celastrol, pharmaceutical compositions comprising these compounds, and uses thereof.

### BACKGROUND

Celastrol, also named tripterine, a naturally occurring compound derived from the plant species, Tripterygium wilfordii Hook F. Celastrol has been found to exhibit significant anti-tumor activity in preclinical studies, including studies for the treatment of liver cancer, breast cancer, prostate cancer, lung cancer, leukemia, melanoma (see e.g., Kashyap et al., 2018; Yadav et al., 2018) and was shown to have radiosensitizing effect (see e.g., Dai et al., 2011; Lee et al., 2011). Results from a range tumor cell lines and animal cancer models have suggested that celastrol can kill tumors via a range of mechanisms of action including: i) induced apoptosis and autophagy, ii) cell cycle arrest, iii) antimetastatic and anti-angiogenic actions, iv) anti-inflammatory effects, and v) antioxidant activities. (see e.g., Cascão et al., 2017; Kashyap et al., 2018). It is believed that celastrol acts by targeting multiple signaling pathways, including but not limited to, reactive oxygen species (ROS)/JNK and Akt/mTOR (see e.g., Liu et al., 2019), NF-κb (see, e.g., Chiang et al., 2014), STAT3/JAK2 (see, e.g., Rajendran et al., 2012), HSP90 (see, e.g., Sreeramulu et al., 2009; Zhang et al., 2009), Cdc37, p23, Iκκb, p-Akt (see e.g., Kannaiyan et al., 2011), ERα (see e.g., Jang et al., 2011).

Celastrol has been shown to act as an inflammasome inhibitor (see e.g., Lee et al., 2019; Yu et al., 2017), and has been proposed for the treatment of a range of inflammatory indications, autoimmune, and several chronic diseases including, but not limited to, rheumatoid arthritis (RA), multiple sclerosis (MS) (see e.g., Wang et al., 2015; Abdin and Hasby, 2014), ankylosing spondylitis, systemic lupus erythematosus (SLE), inflammatory bowel disease, osteoarthritis (OA), acute respiratory distress syndrome (ARDS) (see e.g., Wei and Wang, 2017), Guillain-Barré syndrome (GBS) (e.g. Shao et al., 2023), Sickle cell disease (SCD) (see e.g., Kumar et al., 2016), allergy (e.g. Asthma), psoriasis and other inflammatory skin conditions (see e.g., Vankatesha and Moudgil, 2021; Song et al., 2023).

Celastrol has also been shown to exhibit neuroprotective activity (see e.g., Cascão et al., 2017; Cleren et al, 2005; Paris et al., 2010), and has been proposed for the treatment of a range of neurological disorders, including but not limited to, Parkinson's disease, Huntington disease, Alzheimer's disease, amyotrophic lateral sclerosis (ALS), Gaucher disease (GD) (see e.g., Vankatesha and Moudgil, 2021).

Celastrol, and celastrol analog compounds, have also been proposed as a treatment for multiple metabolic and atherosclerotic diseases, liver diseases, and cardiac disorders, including but not limited to obesity (see e.g., Liu et al., 2015; Feng et al., 2019), atherosclerosis (see e.g., Coll et al., 2015; Voutyritsa et al., 2021), type 2 diabetes (T2D) (see e.g. Liu et al, 2015; Zhou et al., 2021), diabetic nephropathy (see e.g. Nie et al., 2020), gout (Yan et al., 2021; Wen et al., 2014), cardiac fibrosis (see e.g. Fan et al., 2023), non-alcoholic steatohepatitis, hyperlipidemia, hypertension, diabetes, lipodystrophy, fatty liver.

Celastrol was also shown to be useful in the treatment and prevention of multiple brain disorders and injuries due to its anti-inflammatory activities. These include but not limited to middle cerebral artery occlusion (MCAO)-induced brain injury, cerebral ischaemia/reperfusion (I/R) injury, Vascular dementia (VD), and acute ischemic stroke-induced brain injury (see e.g. Jiang et al., 2018).

In recent years more and more application for celastrol were explored for treatment of ocular disorders, this includes but not limited to dry eye disease (see e.g. Siu Law, 2022), ocular inflammation, age-related macular degeneration (AMD) (see e.g., Zhang et al., 2019), subconjunctival fibrosis (see e.g., Li et al., 2023). Celastrol was also shown to protect against the development of ocular hypertension-induced degeneration of retinal ganglion cells (see e.g., Gu et al., 2018), bright light-induced degeneration (see e.g., Bian et al., 2016), and macrophage-induced corneal neovascularization (see e.g., Li et al., 2016), and to support corneal allograft survival (see e.g., Li et al., 2016) and recovery from injuries caused by optic nerve crush (see e.g., Kyung et al., 2015).

Although celastrol interacts with many cellular targets and exhibits strong activities useful for many potential therapeutic indications, the compound exhibits poor ADME characteristics that have limited its further clinical development, including, water stability, low bioavailability, a narrow therapeutic window, and undesired side effects (see e.g., Cascão et al., 2017; Hou et al., 2020).

There exists a need for celastrol derivative or analog compounds that exhibit one or more improved ADME characteristics, while retaining some or all of functional activities of celastrol that are associated with its therapeutic efficacy.

### SUMMARY

The present disclosure relates generally to celastrol derivative compounds, compositions, formulations, and their use in medicaments and therapeutic methods. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a compound of structural formula (I)

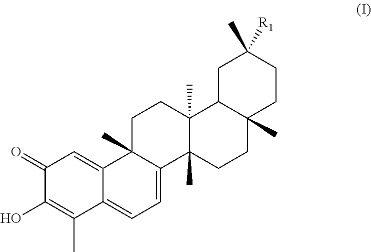

wherein, R₁ is selected from:

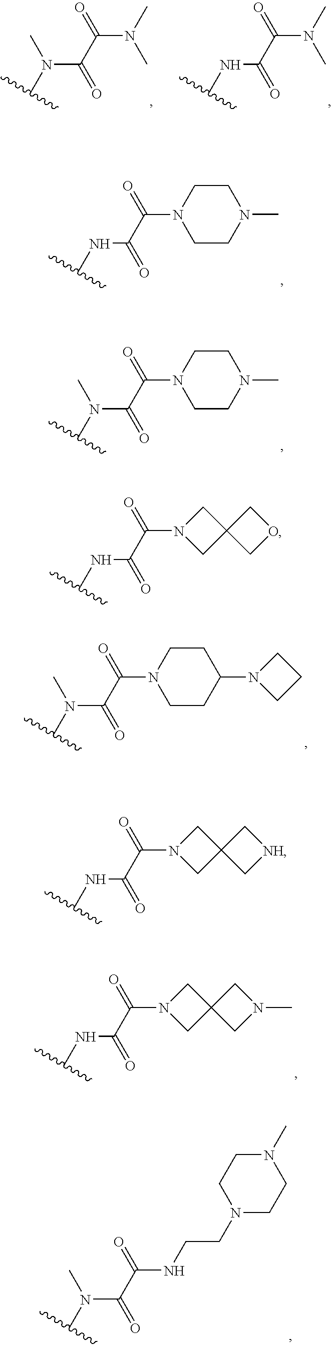

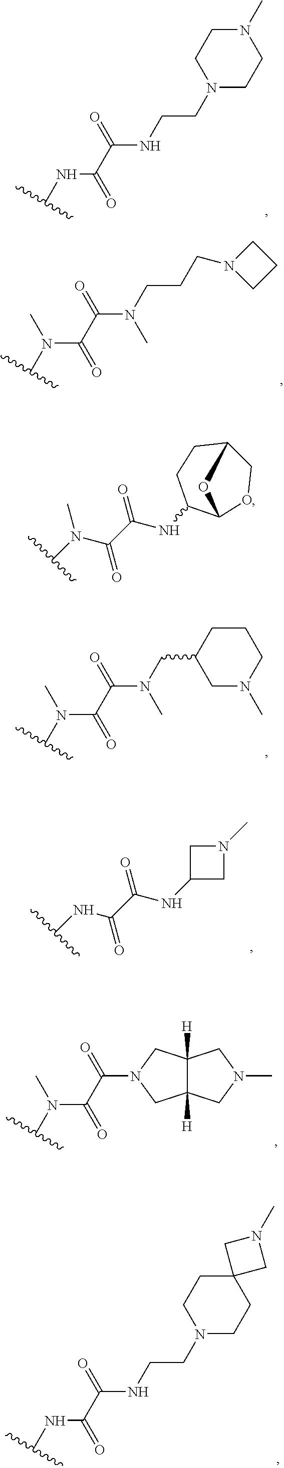

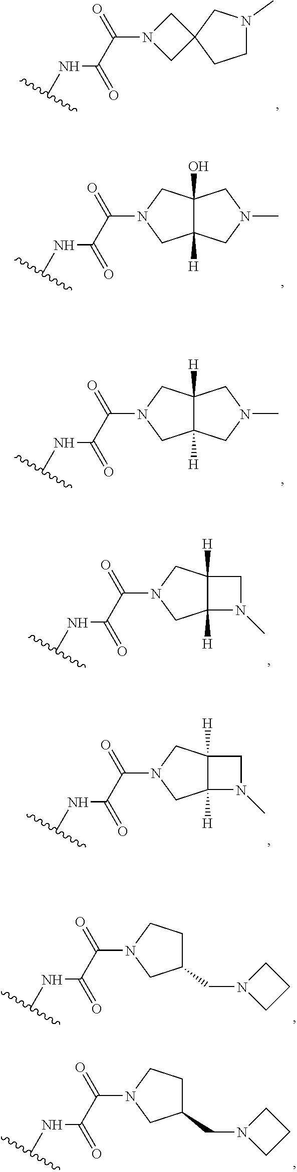

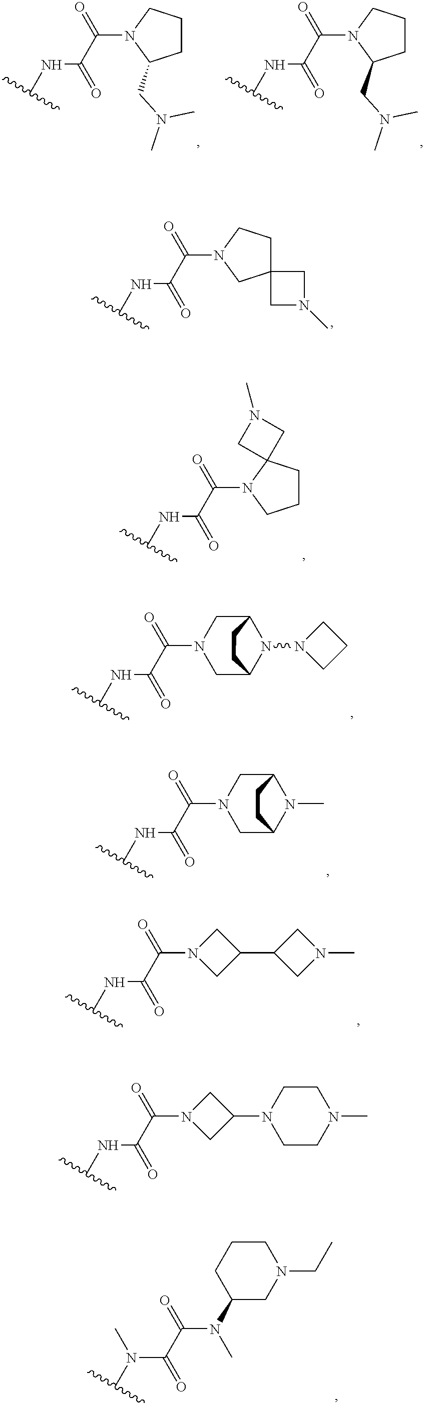

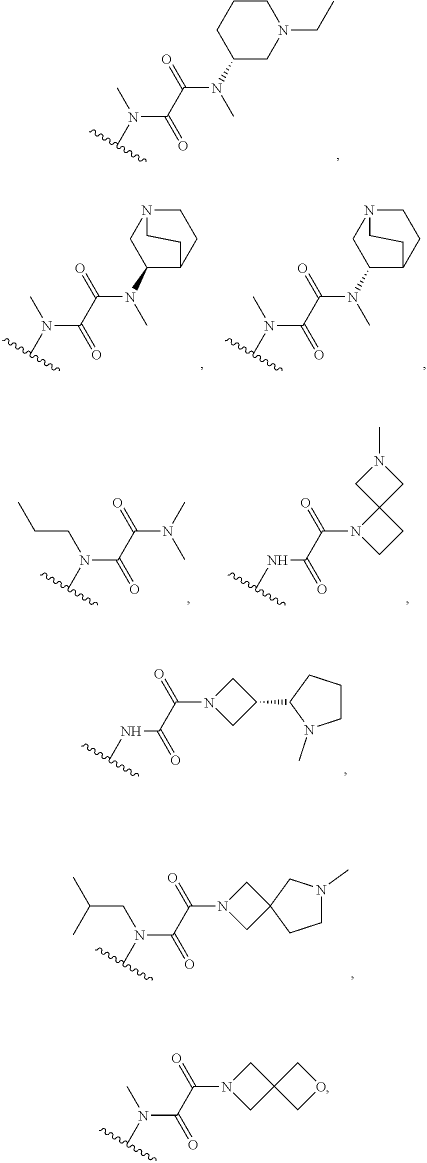

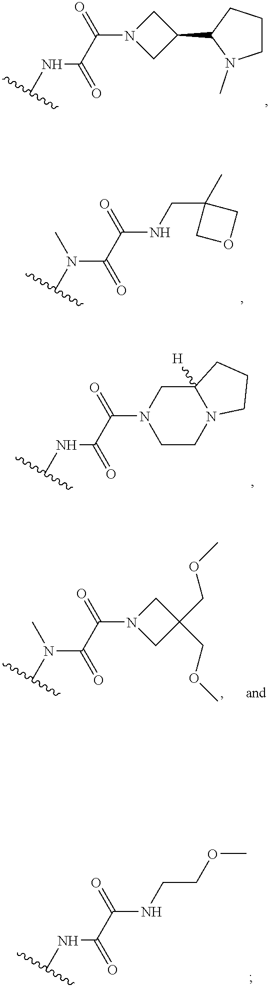

or a pharmaceutically acceptable salt, single stereoisomer, mixture of stereoisomers, tautomer, or prodrug thereof.

In at least one embodiment, the present disclosure provides a compound of structural formula (I)

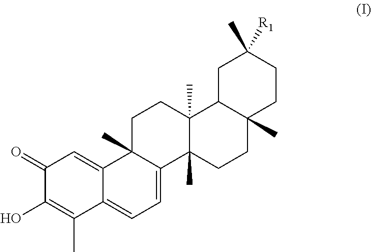

wherein
R₁ is —NH—(CO)₂—NR₂R₃, or —N(CH₃)—(CO)₂—NR₂R₃;
R₂ and R₃ are independently selected from hydrogen, an alkyl, cycloalkyl, alkoxy, heterocycloalkyl, alkylaryl, alkenyl, alkynyl, aryl, amine, or heteroaryl, optionally substituted with substituents individually selected from alkyl, alkoxy, cycloalkyl, ether, amine optionally substituted with one or more alkyl, halogen, hydroxyl, ether, cyano, nitrile, CF₃, ester, amide, cycloalkyl amide, sugar, heteroarylamide optionally substituted with alkyl and/or alkoxy, urea, carbamate, thioether, sulfate, sulfonyl, sulfonic acid carboxylic acid, and aryl; or
R₂ and R₃ taken together form a cycloalkyl, heterocycloalkyl, aryl or heteroaryl group, optionally substituted with substituents individually selected from alkyl, cycloalkyl, alkoxy, heterocycloalkyl, alkylaryl, alkenyl, alkynyl, aryl, heteroaryl, amine, halogen, hydroxyl, ether, nitrile, cyano, nitro, CF₃, ester amide, urea, carbamate, thioether, or carboxylic acid group;
or a pharmaceutically acceptable salt, single stereoisomer, mixture of stereoisomers, tautomer, or prodrug thereof.

In at least one embodiment of any of the compounds of structural formula (I) of the present disclosure, the compound is selected from the group consisting of: compound 14, compound 15, compound 16, compound 17, compound 22, compound 22-1, compound 42, compound 54, compound 55, compound 56, compound 57, compound 58, compound 59, compound 60, compound 61, compound 62, compound 63, compound 64, compound 65, compound 66, compound 67, compound 68, compound 69, compound 70, compound 71, compound 72, compound 73, compound 74, compound 75, compound 76, compound 77, compound 78, compound 79, compound 80, compound 81, compound 82, compound 83, compound 84, compound 85, compound 86, compound 87, compound 88, compound 89, compound 90, compound 91, and compound 92.

In at least one embodiment of any of the compounds of structural formula (I) of the present disclosure, the compound is characterized by having an IC₅₀ in a NF-kB reporter assay of about 1200 nM or less, about 1000 nM or less, 750 nM or less, 500 nM or less, or 300 nM or less.

In at least one embodiment, the present disclosure provides a pharmaceutical composition comprising a compound of structural formula (I) of the present disclosure, and a pharmaceutically acceptable excipient.

In at least one embodiment, the present disclosure provides a method of treating a celastrol-responsive disease or disorder in a subject suffering therefrom, the method comprising administering to the subject a therapeutically effective amount of a compound of structural formula (I) of the present disclosure, or a pharmaceutical composition of the present disclosure.

In at least one embodiment of the method, the celastrol-responsive disease or disorder is a cancer; optionally, wherein the cancer is selected from: gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme (GBM).

In at least one embodiment of the method, the celastrol-responsive disease or disorder is an inflammatory and/or autoimmune disorder; optionally, wherein the inflammatory and/or autoimmune disorder is selected from rheumatoid arthritis (RA), multiple sclerosis (MS), ankylosing spondylitis, systemic lupus erythematosus (SLE), inflammatory bowel disease, osteoarthritis (OA), acute respiratory distress syndrome (ARDS), Guillain-Barre syndrome (GBS), Sickle cell disease (SCD), allergy (e.g. Asthma), psoriasis and other inflammatory skin conditions.

In at least one embodiment of the method, the celastrol-responsive disease or disorder is a neurological disorder; optionally, wherein the neurological disorder is selected from Parkinson's disease, Huntington disease, Alzheimer's disease, amyotrophic lateral sclerosis (ALS), and Gaucher disease (GD).

In at least one embodiment of the method, the celastrol-responsive disease or disorder is an obesity-related disease or disorder; optionally, wherein the obesity-related disease or disorder is selected from obesity, pre-obesity, morbid obesity, type 2 diabetes (T2D), atherosclerosis, diabetic nephropathy, gout, cardiac fibrosis, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome, and MOMO (Macrosomia Obesity Macrocephaly Ocular abnormalities) Syndrome.

In at least one embodiment of the method, the celastrol-responsive disease or disorder is a liver-related disease or disorder; optionally, wherein the liver-related disease or disorder is selected from acute-chronic liver failure (ACLF), alcoholic liver disease, cholestatic liver disease, drug-induced liver disease, hepatocellular carcinoma, non-alcoholic fatty liver disease (NAFLD), non-alcoholic steatohepatitis (NASH), viral hepatitis, and viral liver disease.

In at least one embodiment of the method, the celastrol-responsive disease or disorder is a brain injury or brain disorder optionally, wherein the brain-related injury or disorder is selected from middle cerebral artery occlusion (MCAO)-induced brain injury, cerebral ischemia/reperfusion (I/R) injury, Vascular dementia (VD), and acute ischemic stroke-induced brain injury.

In at least one embodiment of the method, the celastrol-responsive disease or disorder is an ocular disorder or injury optionally, wherein the ocular-related injury or disorder is selected from dry eye disease, ocular inflammation, age-related macular degeneration (AMD), subconjunctival fibrosis, ocular hypertension-induced degeneration of the retina, bright light-induced degeneration, macrophage-induced corneal neovascularization, corneal allograft survival, and optic nerve crush.

In at least one embodiment of the methods of the present disclosure, the compound or pharmaceutical composition is administered in combination with another therapy. In at least one embodiment, administering comprises oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

In at least one embodiment of the methods of the present disclosure, the pharmaceutical composition is administered in a form selected from the group comprising pills, capsules, tablets, granules, powders, salts, crystals, liquid, serums, syrups, suspensions, gels, creams, pastes, films, patches, and vapors.

In at least one embodiment of the methods of the present disclosure, the subject is a mammal. In at least one embodiment, the subject is a human.

In at least one embodiment, the present disclosure provides a use of a compound of structural formula (I) of the present disclosure, or a pharmaceutical composition of the present disclosure for use in therapy, for use as a medicament, for use in treating a celastrol-responsive disease or disorder in a subject, or for the manufacture of a medicament for treating a celastrol-responsive disease or disorder in a subject.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A, FIG. 1B, and FIG. 1C show results illustrating that the celastrol derivative compound 22 (also referred to as “CLM-022”) robustly inhibits NF-κB inflammatory signaling as described in Example 14. FIG. 1A depicts plots of relative NF-κB luciferase activity (RLU) for compound 22 and compound 1 (also referred to as “CLM-001”) assayed at 8-point concentrations (10 μM, 5 μM, 2.5 M, 1.25 μM, 0.63 μM, 0.31 μM, 0.16 μM, 0.016 μM, and 0 μM). FIG. 1B depicts plots of the inhibitory concentrations (IC₅₀) for compounds 22 (CLM-022) and 1 (CLM-001). FIG. 1C shows images of western blots of BMDCs pre-treated with the compounds 1 (CLM-001) or 22 (CLM-022) at the three different concentrations (10 nM, 25 nM, and 50 nM) followed by LPS (500 ng/ml) treatment and assay for the known NF-kB target proteins, iNOS, NLRP3, Cox-2, and IL-1β.

FIG. 2A, FIG. 2B, and FIG. 2C show results illustrating that compound 22 (CLM-022) strongly inhibited IL-1β secretion via suppression of NLRP3 inflammasome in bone marrow-derived dendritic cells (BMDCs) as described in Example 15. FIG. 2A depicts western blot images showing the relative NLRP3 inflammasome activity of the compounds 22 (CLM-022) and 1 (CLM-001) by detecting the level of cellular ASC complex and the mature form of IL-1β (15 kDa) in a conditioned media of BMDCs treated with LPS (100 ng/ml) for 3 hours and then the compound at 50 nM, 100 nM, and 500 nM concentration for 30 min, followed by ATP or Nigericin. ASC monomer and β-Actin proteins were used as controls. FIG. 2B depict plots of results showing secreted IL-1β levels measured by ELISA using conditioned media of BMDC treated with the compound 22 or compound 1 at 63 nM, 125 nM, 250 nM, and 500 nM, under NLRP3 inflammasome activation induced either by LPS+ATP or LPS+Nigericin. FIG. 2C depict plots of results showing secreted TNF-α levels measured by ELISA using conditioned media of BMDC treated with the compound 22 or compound 1 at 63 nM, 125 nM, 250 nM, and 500 nM, under NLRP3 inflammasome activation induced either by LPS+ATP or LPS+Nigericin.

FIG. 3A, and FIG. 3B depict results showing compound 22 (CLM-022) inhibited NLRP3 inflammasome activity in THP1 and the cellular inflammasome complex as visualized by Amnis analysis as described in Example 16. FIG. 3A shown images of western blot analysis of THP1 cells treated with LPS, and compound 1 (CLM-001) or compound 22, followed by Nigericin for NLRP3 inflammasome activation. The inflammasome ASC complex in the control cells (LPS+Nigericin) was compared with the treatment of compound 1 (CLM-001) (50 nM, 100 nM), compound 22 (CLM-022) (50 nM, 100 nM), and MCC950 (100 nM, 200 nM). FLICA (green) represents active caspase-1, ASC (red) complex is shown as a red speckle near the nucleus membrane, and DAPI stain for nucleus (blue) captured in the AMNIS analysis. FIG. 3B depicts the cell numbers stained as ASC+ (red) and FLICA+ (green) in the AMNIS were counted in order to compare the level of NLRP3 inflammasome activity in the cells treated as indicated (left). The ASC+ and FLICA+ cells were also plotted as a percentage of total cell count (right).

FIG. 4A, and FIG. 4B depict results showing that compound 22 (CLM-022) decreased the production of pro-inflammatory cytokines in the LPS-induced septic shock mouse model as described in Example 17. Compound 1 (CLM-001) and compound 22 (CLM-022) (1 mg/kg) injected intraperitoneally 1 hour prior to septic shock induction by administering sublethal dose of LPS (25 mg/kg, i.p.), mice were sacrificed 4 hours post LPS injection, and subjected to measurement of inflammatory cytokine expression. FIG. 4A depicts plot of data showing that the level of TNF-α decreased in the serum of compound 1 and compound 22 treated mice. FIG. 4B depicts plots showing results of gene expression analysis performed by qPCR have shown that compound 22 pretreatment markedly inhibited for IL-β, IL-6, TNF-α, and iNOS inflammatory cytokines and molecule in the colon.

FIG. 5A, FIG. 5B, FIG. 5C and FIG. 5D depict results showing that compound 22 (CLM-022) outperforms the benchmark inflammasome inhibitor compound, MCC950, in inhibiting IL-1β secretion (but not TNF-α secretion) in BMDM cells and THP-1 cells activated for inflammasome activity and cytokine secretion by LPS and Nigericin treatment as described in Example 18.

FIG. 6A, FIG. 6B, and FIG. 6C depict results of the DARTS assays described in Example 19 showing that the celastrol derivative, compound 22 (CLM-022), when compared to MCC950, exhibits markedly improved protection of NLRP3 protein, but not NEK7, from Pronase-induced degradation.

FIG. 7A, FIG. 7B, and FIG. 7C depict results of a study described in Example 20, which show that the celastrol derivative, compound 22 (CLM-022), when compared to MCC950, possesses a greater capacity to inhibit inflammasome-induced pyroptosis in human THP-1 macrophages.

### DETAILED DESCRIPTION

It is to be understood that the detailed descriptions provided herein, including the drawings, are exemplary and explanatory only and are not restrictive of this disclosure. The description is not limited to the specific compounds, compositions, methods, techniques, protocols, cell lines, assays, and reagents disclosed herein, as these may vary, but is also intended to encompass known variants of these specific embodiments.

It is also to be understood that the terminology used herein is intended to describe particular embodiments and is in not intended to limit the scope as set forth in the appended claims. For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the present disclosure. The upper and lower limits of these smaller ranges may independently be included in the smaller ranges, and are also encompassed within the present disclosure, subject to any specifically excluded limit in the stated range. Where the stated range includes one or both of these limits, ranges excluding (i) either or (ii) both of those included limits are also included in the present disclosure. For example, “1 to 50,” includes “2 to 25,” “5 to 20,” “25 to 50,” “1 to 10,” etc.

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present disclosure pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### I. Definitions

“Celastrol” refers to a compound having a chemical structure of compound (1):

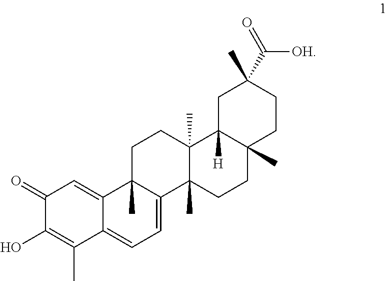

“Celastrol-responsive disease or disorder” refers to any disease or disorder for which treatment with the compound, celastrol has been proposed or shown to provide a potentially therapeutic effect, based on clinical, pre-clinical, or in vitro studies. Exemplary celastrol-responsive diseases or disorders of the present disclosure include, but are not limited to, cancer (e.g., gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme), inflammatory and/or autoimmune disorders (e.g., rheumatoid arthritis (RA) multiple sclerosis (MS) and experimental autoimmune encephalomyelitis (EAE), ankylosing spondylitis, systemic lupus erythematosus (SLE), Ulcerative colitis, inflammatory bowel disease, osteoarthritis (OA), acute respiratory distress syndrome (ARDS), Guillain-Barré syndrome (GBS), Sickle cell disease (SCD), Asthma, psoriasis and, and skin inflammation), neurological disorders (e.g., Parkinson's disease, Huntington disease, Alzheimer's disease, amyotrophic lateral sclerosis (ALS), and Gaucher disease (GD)), and obesity-related disorders (e.g., obesity, pre-obesity, morbid obesity, type 2 diabetes (T2D), atherosclerosis, diabetic nephropathy, gout, cardiac fibrosis, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome and MOMO (Macrosomia Obesity Macrocephaly Ocular abnormalities) Syndrome), brain injuries and disorders (e.g., middle cerebral artery occlusion (MCAO)-induced brain injury, cerebral ischaemia/reperfusion (I/R) injury, Vascular dementia (VD), and acute ischemic stroke-induced brain injury), and ocular disorders (e.g., dry eye disease, ocular inflammation, age-related macular degeneration (AMD), subconjunctival fibrosis, ocular hypertension-induced degeneration of the retina, bright light-induced degeneration, macrophage-induced corneal neovascularization, corneal allograft survival, and optic nerve crush).

The term “optional” or “optionally” mean that the subsequently described event or circumstance may or may not occur, and that the description includes instances where said event or circumstance occurs and instances in which it does not.

The term “substituted” includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (or “moiety”) is described herein as being attached to a second group and the site of attachment is not explicit, the given group may be attached at any available site of the given group to any available site of the second group. For example, a “lower alkyl-substituted phenyl,” where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to substituted aryl-(substituted aryl)-substituted aryl.

A compound of a given formula (e.g. the “compound of Formula (I)”) is intended to encompass the compounds of the disclosure, and the pharmaceutically acceptable salts, pharmaceutically acceptable esters, hydrates, polymorphs, and prodrugs of such compounds.

Additionally, the compounds of the disclosure may possess one or more asymmetric centers and can be produced as a racemic mixture or as individual enantiomers or diastereoisomers. The number of stereoisomers present in any given compound of a given Formula depends upon the number of asymmetric centers present (there are 2n stereoisomers possible where n is the number of asymmetric centers). The individual stereoisomers may be obtained by resolving a racemic or non-racemic mixture of an intermediate at some appropriate stage of the synthesis, or by resolution of the compound by conventional means. The individual stereoisomers (including individual enantiomers and diastereoisomers) as well as racemic and non-racemic mixtures of stereoisomers are encompassed within the scope of the present disclosure, all of which are intended to be depicted by the structures of this specification unless otherwise specifically indicated.

The term “isomers” means different compounds that have the same molecular formula. Isomers include stereoisomers, enantiomers, and diastereomers. The term “stereoisomers” means isomers that differ only in the way the atoms are arranged in space. The term “enantiomers” means a pair of stereoisomers that are non-superimposable mirror images of each other. A 1:1 mixture of a pair of enantiomers is a “racemic” mixture. The term “(±)” is used to designate a racemic mixture where appropriate. The term “diastereoisomers” means stereoisomers that have at least two asymmetric atoms, but which are not mirror-images of each other. Absolute stereochemistry is specified herein according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or levorotary) that they rotate the plane of polarized light at the wavelength of the sodium D line.

Certain compounds of the present disclosure include asymmetric atoms (optical or chiral centers) or double bonds. The present disclosure is intended to encompass within the scope of the compounds described herein, even where those compounds described by chemical structures and formulas that do not explicitly show the various stereoisomeric or diastereomeric forms, including the racemic or optically pure forms of the compounds.

Optically active (R)- and (S)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

“Tautomeric isomers” or “tautomers” are isomers that are in equilibrium with one another. For example, amide containing compounds may exist in equilibrium with imidic acid tautomers. Certain compounds of the present disclosure exist as ‘tautomeric isomers” or “tautomers.” Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers.

The term “polymorph” refers to different crystal structures of a crystalline compound. The different polymorphs may result from differences in crystal packing (packing polymorphism) or differences in packing between different conformers of the same molecule (conformational polymorphism). Certain compounds of the present disclosure may exist in multiple crystalline or amorphous forms. Generally, all physical forms are equivalent for the uses contemplated by the present disclosure and are intended to be within the scope of the disclosure.

The term “solvate” refers to a complex formed by combining a compound and a solvent. The term “hydrate” refers to the complex formed by combining a compound and water. Certain compounds of the present disclosure can exist in unsolvated forms, solvated forms, which includes hydrated forms. Generally, the solvated forms are equivalent to the unsolvated forms and are intended to be encompassed within the scope of the compounds described herein, even where those compounds are described by chemical structures and formulas that do not explicitly show the solvated form.

The term “salt” as used herein refers to ionic compounds that result from the neutralization reaction of an acid and a base. Salts are composed of related numbers of cations (positively charged ions) and anions (negative ions) so that the product is neutral (without a net charge). These component ions can be inorganic, such as chloride (Cl⁻), or organic, such as acetate (C₂H₃O₂⁻); and can be monatomic, such as fluoride (F⁻), or polyatomic, such as sulfate (SO₄²⁻).

The term “pharmaceutically acceptable salt” of a given compound refers to salts that retain the biological effectiveness and properties of the given compound, and which are not biologically or otherwise undesirable. In many cases, the compounds of this disclosure are capable of forming pharmaceutically acceptable acid and/or base salts by virtue of the presence of amino and/or carboxyl groups or groups similar thereto.

Pharmaceutically acceptable base addition salts can be prepared from inorganic and organic bases. Salts derived from inorganic bases include, by way of example only, sodium, potassium, lithium, ammonium, calcium, and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Specific examples of suitable amines include, by way of example only, isopropylamine, trimethyl amine, diethyl amine, tri(iso-propyl)amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts also may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “pharmaceutically acceptable carrier” or “pharmaceutically acceptable excipient” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Any formula or structure given herein, including Formula (I), is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as ³H, ¹³C, and ¹⁴C are incorporated. Such isotopically labelled compounds may be useful in metabolic studies, reaction kinetic studies, detection, or imaging techniques, such as positron emission tomography (PET) or single-photon emission computed tomography (SPECT) including drug or substrate tissue distribution assays, or in radioactive treatment of patients.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula (I).

The concentration of such a heavier isotope, specifically deuterium, may be defined by an isotopic enrichment factor. In the compounds of the present disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In the description, including the Examples, all temperatures are in degrees Celsius (C), unless otherwise stated, and abbreviations and acronyms have the following meanings listed in Table 1 (below).

TABLE 1

| Abbreviation | Meaning |
|---|---|
| AcOH | Acetic acid |
| BHA | Butylated hydroxy anisole |
| Boc | tert-Butoxycarbonyl |
| (Boc)₂O | Di(tert-butyl) carbonate |
| °C | Degree Celsius |
| Cbz | Benzyl carbamate |
| cm | centimeter |
| Cs₂CO₃ | Cesium carbonate |
| DBU | 1,8-Diazabicyclo[5.4.0]undec-7-ene |
| DCC | Dicyclohexyl carbodiimide |
| DCM | Dichloromethane |
| DIC | Diisopropyl carbodiimide |
| DIEA | N,N-Diisopropylethylamine |
| DIPEA | Diisopropylethylamine |
| DMAP | 4-Dimethylaminopyridine |
| DMF | Dimethylformamide |
| DMF | Dimehtylformamide |
| DMSO | Dimethylsulfoxide |
| EDTA | Ethylenediaminetetraacetic acid |
| equiv/eq | Equivalents |
| EtOAc | Ethyl acetate |
| EtOH | Ethanol |
| g | Grams |
| HATU | (1-[Bis(dimethylamino)methylene]-1H-1,2,3-triazolo[4,5-b]pyridinium |
| | 3-oxide hexafluorophosphate |
| HPLC | High-performance liquid chromatography |
| hrs/h | Hours |
| Hz | Hertz |
| IC₅₀ | The half maximal inhibitory concentration |
| IIDQ | 1-Isobutoxycarbonyl-2-isobutoxy-1,2-dihydro quinone |
| ip | Intraperitoneal |
| iv | Intravenous |
| J | Coupling constant |
| K₂CO₃ | Potassium carbonate |
| Kg | Kilogram |
| KOAc | Potassium acetate |
| KOtBu | Potassium tert-Butoxide |
| L | Liter |
| LC-MS | Liquid chromatography-mass spectrometry |
| LG | Leaving group |
| M | Molar |
| m/z | mass-to-charge ratio |
| M+ | Mass peak |
| M + H | Mass peak plus hydrogen |
| M + Na | Mass peak plus sodium |
| Me | Methyl |
| MeOH | Methanol |
| mg | Milligram |
| MHz | Megahertz |
| min | Minute |
| mL | Milliliter |
| mM | Millimolar |
| mmol | Millimole |
| MS | Mass spectroscopy |
| MTBE | Methyl tert-butyl ether |
| Na₂CO₃ | Sodium carbonate |
| NaBH₄ | Sodium borohydride |
| NAD | Nicotinamide Adenine Dinucleotide |
| NaOH | Sodium hydroxide |
| NMM | N-Methylmorpholine |
| NMP | N-Methyl-2-pyrrolidone |
| PG | Protecting group |
| Ph | Phenyl |
| q.s. | Quantity sufficient to achieve a stated function |
| RT or rt | Room temperature |
| s | Second |
| sc | Subcutaneous |
| TEA | Triethylamine |
| THF | Tetrahydrofuran |
| TLC | Thin layer chromatography |
| TMS | Trimethylsilyl |
| TMSCl | Trimethysilyl chloride |
| Tris | tris(hydroxymethyl)aminomethane |
| δ | Chemical shift |
| μg | Microgram |
| μL (or μl) | Microliter |
| μM | Micromolar |
| μmol | Micromole |

### II. Celastrol Derivative Compounds

The present disclosure provides a range of compounds having chemical structures that are derivatives of the structure of celastrol (compound (1)). Generally, the celastrol derivative compounds of the present disclosure retain the 5-ring tripterine core structure of compound (1) and replace the carboxylic acid group with a range of different substituted amine and amide groups as described in greater detail below. The celastrol derivative compounds of the present disclosure retain or improve upon the functional features of celastrol including NF-κB cell inhibition and ADME properties, thereby providing alternative molecules for use in treating celastrol-responsive diseases and disorders. Accordingly, in at least one embodiment, the celastrol derivative compounds of the present disclosure include those compounds of structural formula (I)

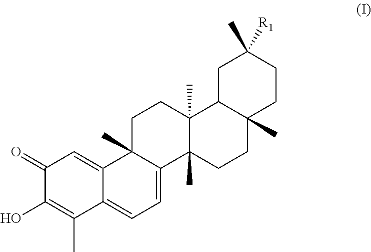

wherein the R₁ group (or moiety) of the derivative compound is the group that replaces the carboxylic acid group of compound 1. The present disclosure contemplates a range of possible chemical groups for R₁ including those listed in Table 2 (below).

TABLE 2
Exemplary Celastrol Derivative of Formula (I) R₁ Groups

| | |
|---|---|
| 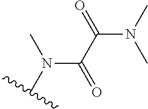 [SMILES: *N(C)C(=O)C(=O)N(C)C] | 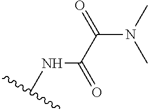 [SMILES: *NC(=O)C(=O)N(C)C] |
| 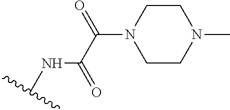 [SMILES: *NC(=O)C(=O)N1CCN(C)CC1] | 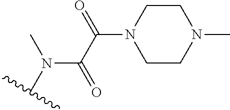 [SMILES: *N(C)C(=O)C(=O)N1CCN(C)CC1] |
| 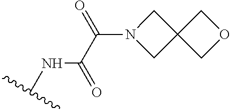 [SMILES: *NC(=O)C(=O)N1CC2(COC2)C1] | 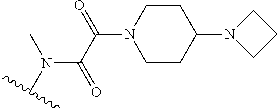 [SMILES: *N(C)C(=O)C(=O)N1CCC(N2CCC2)CC1] |
| 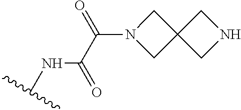 [SMILES: *NC(=O)C(=O)N1CC2(CNC2)C1] | 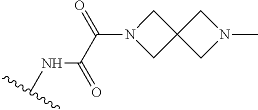 [SMILES: *NC(=O)C(=O)N1CC2(CN(C)C2)C1] |
| 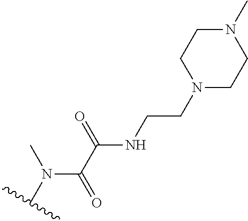 [SMILES: *N(C)C(=O)C(=O)NCCN1CCN(C)CC1] | 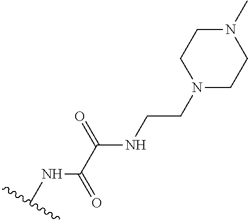 [SMILES: *NC(=O)C(=O)NCCN1CCN(C)CC1] |
| 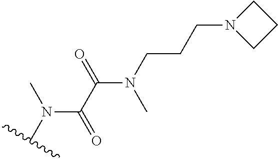 [SMILES: *N(C)C(=O)C(=O)N(C)CCCN1CCC1] | 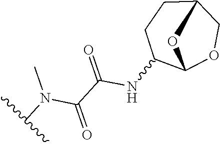 [SMILES: *N(C)C(=O)C(=O)NC1CC[C@@H]2CO[C@H]1O2] |
| 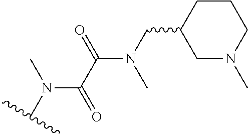 [SMILES: *N(C)C(=O)C(=O)N(C)CC1CCCN(C)C1] | 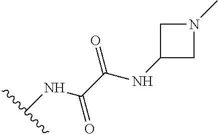 [SMILES: *NC(=O)C(=O)NC1CN(C)C1] |
| 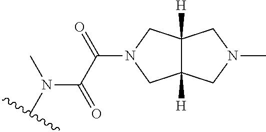 [SMILES: *N(C)C(=O)C(=O)N1C[C@H]2CN(C)C[C@H]2C1] | 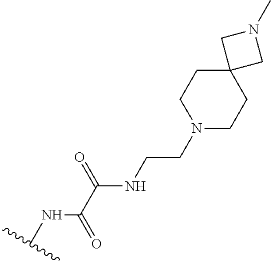 [SMILES: *NC(=O)C(=O)NCCN1CCC2(CC1)CN(C)C2] |
| 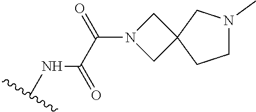 [SMILES: *NC(=O)C(=O)N1CC2(CCN(C)C2)C1] | 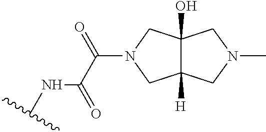 [SMILES: *NC(=O)C(=O)N1C[C@H]2CN(C)C[C@@]2(O)C1] |
| 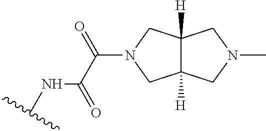 [SMILES: *NC(=O)C(=O)N1C[C@@H]2CN(C)C[C@H]2C1] | 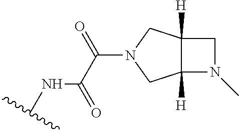 [SMILES: *NC(=O)C(=O)N1C[C@@H]2CN(C)[C@@H]2C1] |
| 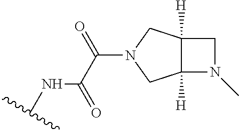 [SMILES: *NC(=O)C(=O)N1C[C@H]2CN(C)[C@H]2C1] | 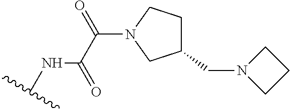 [SMILES: *NC(=O)C(=O)N1CC[C@H](CN2CCC2)C1] |
| 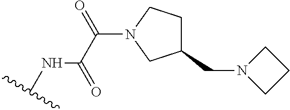 [SMILES: *NC(=O)C(=O)N1CC[C@@H](CN2CCC2)C1] | 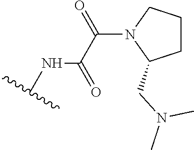 [SMILES: *NC(=O)C(=O)N1CCC[C@@H]1CN(C)C] |
| 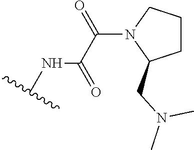 [SMILES: *NC(=O)C(=O)N1CCC[C@H]1CN(C)C] | 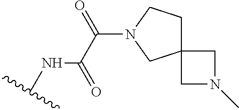 [SMILES: *NC(=O)C(=O)N1CCC2(CN(C)C2)C1] |
| 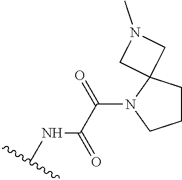 [SMILES: *NC(=O)C(=O)N1CCCC12CN(C)C2] | 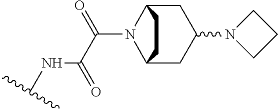 |
| 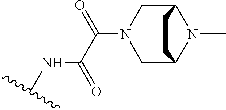 | 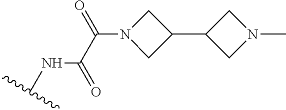 [SMILES: *NC(=O)C(=O)N1CC(C2CN(C)C2)C1] |
| 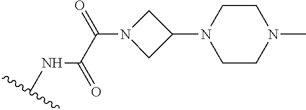 [SMILES: *NC(=O)C(=O)N1CC(N2CCN(C)CC2)C1] | 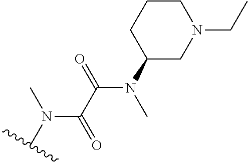 [SMILES: *N(C)C(=O)C(=O)N(C)[C@H]1CCCN(CC)C1] |
| 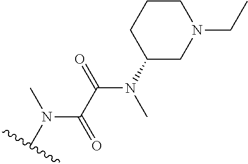 [SMILES: *N(C)C(=O)C(=O)N(C)[C@@H]1CCCN(CC)C1] | 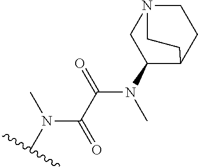 [SMILES: *N(C)C(=O)C(=O)N(C)[C@H]1CN2CCC1CC2] |
| 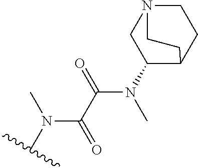 [SMILES: *N(C)C(=O)C(=O)N(C)[C@@H]1CN2CCC1CC2] | 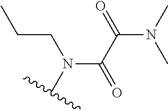 [SMILES: *N(CCC)C(=O)C(=O)N(C)C] |
| 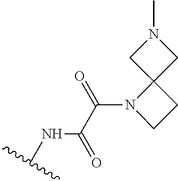 [SMILES: *NC(=O)C(=O)N1CCC12CN(C)C2] | 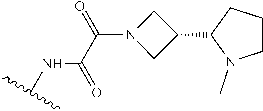 |
| 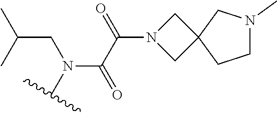 [SMILES: *N(CC(C)C)C(=O)C(=O)N1CC2(CCN(C)C2)C1] | 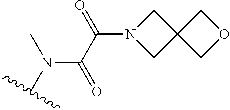 [SMILES: *N(C)C(=O)C(=O)N1CC2(COC2)C1] |
| 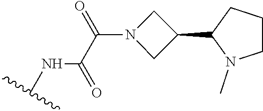 | 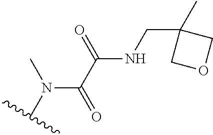 [SMILES: *N(C)C(=O)C(=O)NCC1(C)COC1] |
| 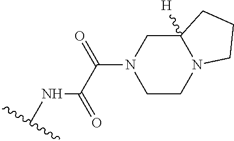 [SMILES: *NC(=O)C(=O)N1CCN2CCCC2C1] | 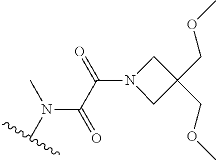 [SMILES: *N(C)C(=O)C(=O)N1CC(COC)(COC)C1] |
| 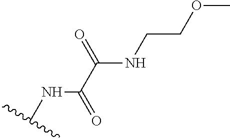 [SMILES: *NC(=O)C(=O)NCCOC] | |

The present disclosure also contemplates that the celastrol derivative compounds of structural formula (I) can include selected sub-genera of compounds. For example, in at least one embodiment, the present disclosure provides celastrol derivative compounds of structural formula (I), where the group R₁ can be —NH—(CO)₂—NR₂R₃, or —N(CH₃)—(CO)₂—NR₂R₃. In at least one embodiment of this sub-genus of compounds, the groups R₂ and R₃ of the structural formula can be independently selected from a hydrogen, an alkyl, cycloalkyl, alkoxy, heterocycloalkyl, alkylaryl, alkenyl, alkynyl, aryl, amine, or heteroaryl, optionally substituted with substituents individually selected from alkyl, alkoxy, cycloalkyl, ether, amine optionally substituted with one or more alkyl, halogen, hydroxyl, ether, cyano, nitrile, CF₃, ester, amide, cycloalkyl amide, sugar, heteroarylamide optionally substituted with alkyl and/or alkoxy, urea, carbamate, thioether, sulfate, sulfonyl, sulfonic acid carboxylic acid, and aryl. In another embodiment of this sub-genus, the groups R₂ and R₃ of the structural formula can be taken together so as to form a cycloalkyl, heterocycloalkyl, aryl or heteraryl group, optionally substituted with substituents individually selected from alkyl, cycloalkyl, alkoxy, heterocycloalkyl, alkylaryl, alkenyl, alkynyl, aryl, heteroaryl, amine, halogen, hydroxyl, ether, nitrile, cyano, nitro, CF₃, ester amide, urea, carbamate, thioether, or carboxylic acid group.

Exemplary celastrol derivative compounds of the present disclosure are provided in Table 3 below. The synthesis, characterization, and uses of these exemplary compounds are described in the Examples and elsewhere herein.

TABLE 3
Exemplary Celastrol Derivative Compounds

| | | Molecular |
|---|---|---|
| Compound | | Weight |
| Number | Structure | (m/e) |
| 14 | 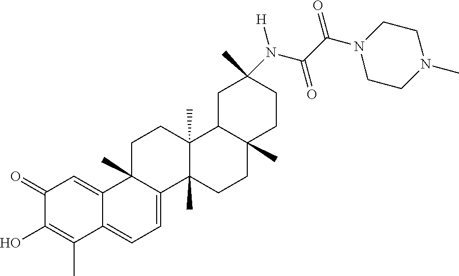 [SMILES: CC1=C(O)C(=O)C=C2C1=CC=C1[C@@]2(C)CC[C@@]2(C)C3C[C@](C)(NC(=O)C(=O)N4CCN(C)CC4)CC[C@]3(C)CC[C@]12C] | 576 |
| 15 | 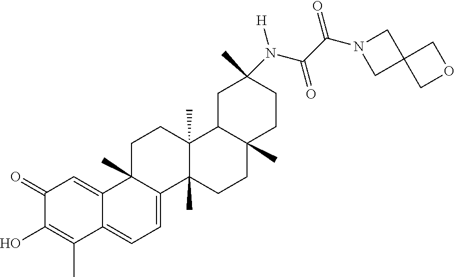 [SMILES: CC1=C(O)C(=O)C=C2C1=CC=C1[C@@]2(C)CC[C@@]2(C)C3C[C@](C)(NC(=O)C(=O)N4CC5(COC5)C4)CC[C@]3(C)CC[C@]12C] | 575 |
| 16 | 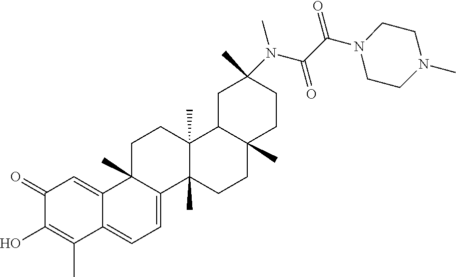 [SMILES: CC1=C(O)C(=O)C=C2C1=CC=C1[C@@]2(C)CC[C@@]2(C)C3C[C@](C)(N(C)C(=O)C(=O)N4CCN(C)CC4)CC[C@]3(C)CC[C@]12C] | 590 |
| 17 | 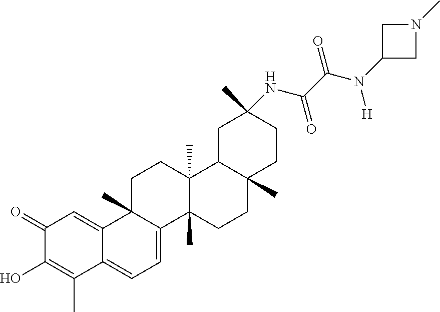 [SMILES: CC1=C(O)C(=O)C=C2C1=CC=C1[C@@]2(C)CC[C@@]2(C)C3C[C@](C)(NC(=O)C(=O)NC4CN(C)C4)CC[C@]3(C)CC[C@]12C] | 562 |
| 22 | 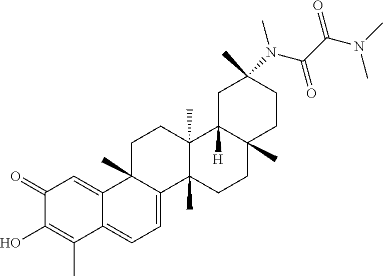 | 521 |
| 22-1 | 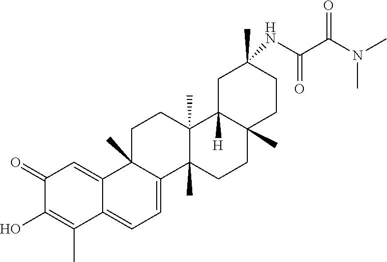 | 507 |
| 42 | 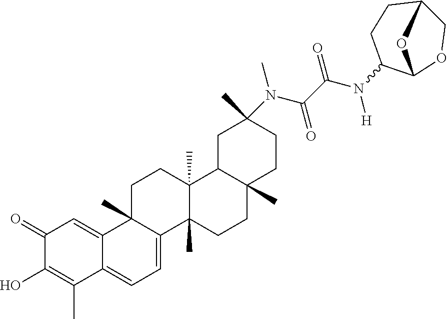 [SMILES: CC1=C(O)C(=O)C=C2C1=CC=C1[C@@]2(C)CC[C@@]2(C)C3C[C@](C)(N(C)C(=O)C(=O)NC4CC[C@@H]5CO[C@H]4O5)CC[C@]3(C)CC[C@]12C] | 619 |
| 54 | 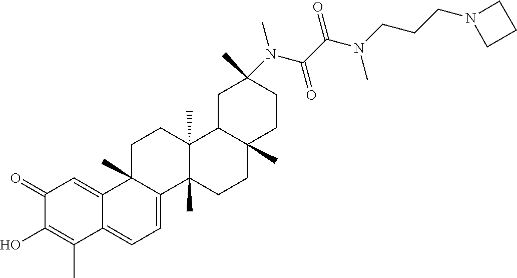 [SMILES: CC1=C(O)C(=O)C=C2C1=CC=C1[C@@]2(C)CC[C@@]2(C)C3C[C@](C)(N(C)C(=O)C(=O)N(C)CCCN4CCC4)CC[C@]3(C)CC[C@]12C] | 618 |
| 55 | 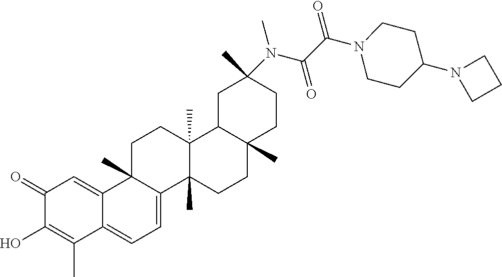 [SMILES: CC1=C(O)C(=O)C=C2C1=CC=C1[C@@]2(C)CC[C@@]2(C)C3C[C@](C)(N(C)C(=O)C(=O)N4CCC(N5CCC5)CC4)CC[C@]3(C)CC[C@]12C] | 630 |
| 56 | 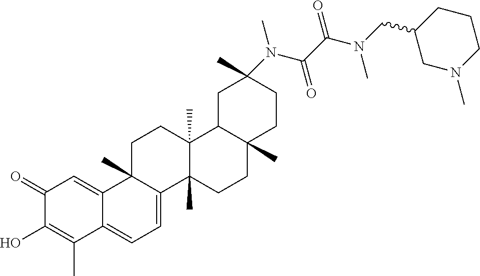 [SMILES: CC1=C(O)C(=O)C=C2C1=CC=C1[C@@]2(C)CC[C@@]2(C)C3C[C@](C)(N(C)C(=O)C(=O)N(C)CC4CCCN(C)C4)CC[C@]3(C)CC[C@]12C] | 632 |
| 57 | 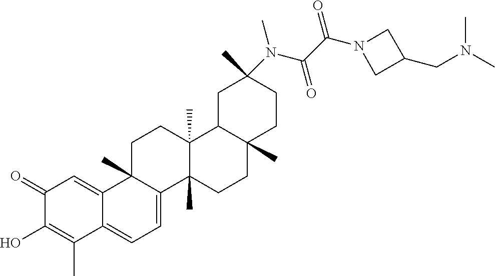 [SMILES: CC1=C(O)C(=O)C=C2C1=CC=C1[C@@]2(C)CC[C@@]2(C)C3C[C@](C)(N(C)C(=O)C(=O)N4CC(CN(C)C)C4)CC[C@]3(C)CC[C@]12C] | 604 |
| 58 | 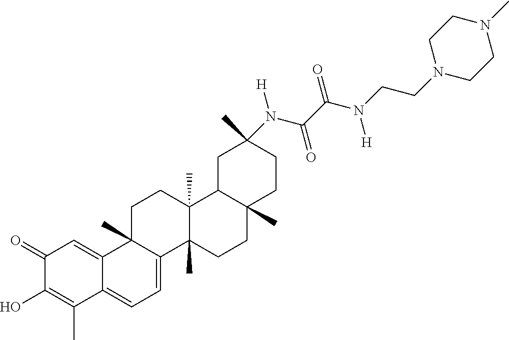 [SMILES: CC1=C(O)C(=O)C=C2C1=CC=C1[C@@]2(C)CC[C@@]2(C)C3C[C@](C)(NC(=O)C(=O)NCCN4CCN(C)CC4)CC[C@]3(C)CC[C@]12C] | 619 |
| 59 | 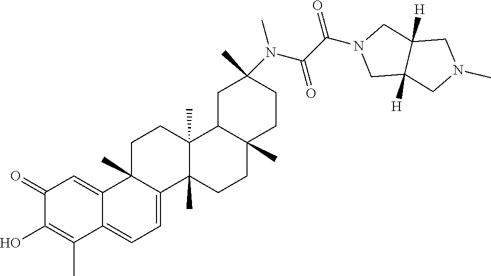 [SMILES: CC1=C(O)C(=O)C=C2C1=CC=C1[C@@]2(C)CC[C@@]2(C)C3C[C@](C)(N(C)C(=O)C(=O)N4C[C@H]5CN(C)C[C@H]5C4)CC[C@]3(C)CC[C@]12C] | 616 |
| 60 | 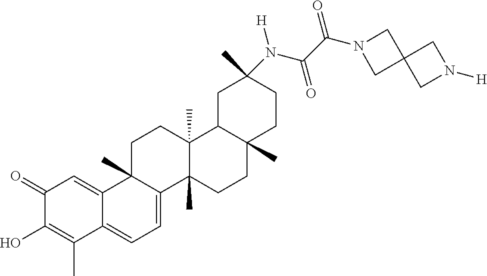 [SMILES: CC1=C(O)C(=O)C=C2C1=CC=C1[C@@]2(C)CC[C@@]2(C)C3C[C@](C)(NC(=O)C(=O)N4CC5(CNC5)C4)CC[C@]3(C)CC[C@]12C] | 574 |
| 61 | 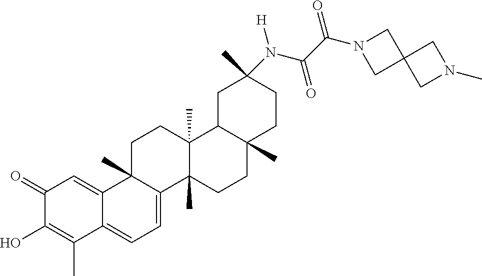 [SMILES: CC1=C(O)C(=O)C=C2C1=CC=C1[C@@]2(C)CC[C@@]2(C)C3C[C@](C)(NC(=O)C(=O)N4CC5(CN(C)C5)C4)CC[C@]3(C)CC[C@]12C] | 588 |
| 62 | 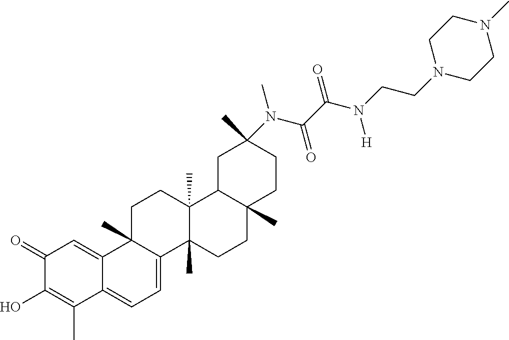 [SMILES: CC1=C(O)C(=O)C=C2C1=CC=C1[C@@]2(C)CC[C@@]2(C)C3C[C@](C)(N(C)C(=O)C(=O)NCCN4CCN(C)CC4)CC[C@]3(C)CC[C@]12C] | 633 |
| 63 | 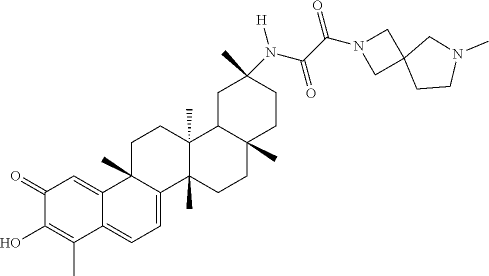 [SMILES: CC1=C(O)C(=O)C=C2C1=CC=C1[C@@]2(C)CC[C@@]2(C)C3C[C@](C)(NC(=O)C(=O)N4CC5(CCN(C)C5)C4)CC[C@]3(C)CC[C@]12C] | 602 |
| 64 | 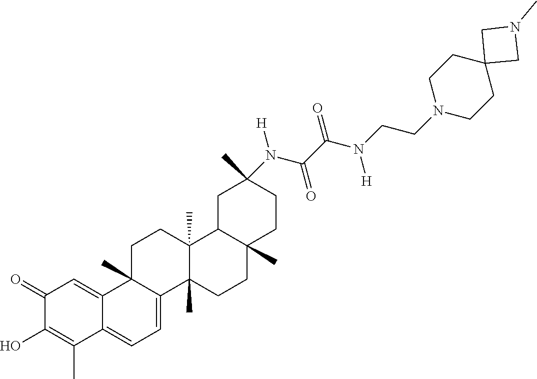 [SMILES: CC1=C(O)C(=O)C=C2C1=CC=C1[C@@]2(C)CC[C@@]2(C)C3C[C@](C)(NC(=O)C(=O)NCCN4CCC5(CC4)CN(C)C5)CC[C@]3(C)CC[C@]12C] | 659 |
| 65 | 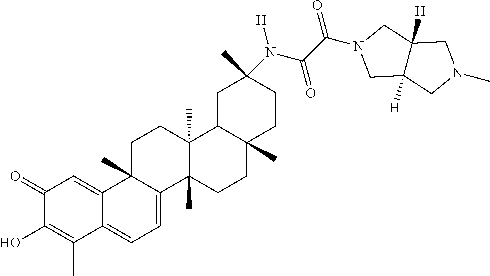 [SMILES: CC1=C(O)C(=O)C=C2C1=CC=C1[C@@]2(C)CC[C@@]2(C)C3C[C@](C)(NC(=O)C(=O)N4C[C@@H]5CN(C)C[C@H]5C4)CC[C@]3(C)CC[C@]12C] | 602 |
| 66 | 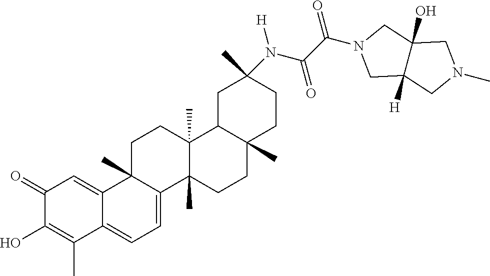 [SMILES: CC1=C(O)C(=O)C=C2C1=CC=C1[C@@]2(C)CC[C@@]2(C)C3C[C@](C)(NC(=O)C(=O)N4C[C@H]5CN(C)C[C@@]5(O)C4)CC[C@]3(C)CC[C@]12C] | 618 |
| 67 | 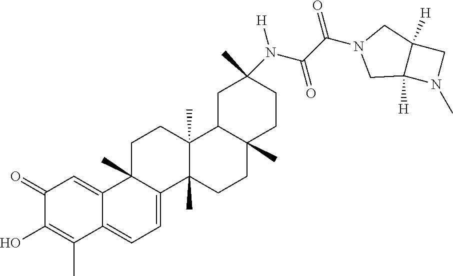 [SMILES: CC1=C(O)C(=O)C=C2C1=CC=C1[C@@]2(C)CC[C@@]2(C)C3C[C@](C)(NC(=O)C(=O)N4C[C@H]5CN(C)[C@H]5C4)CC[C@]3(C)CC[C@]12C] | 588 |
| 68 | 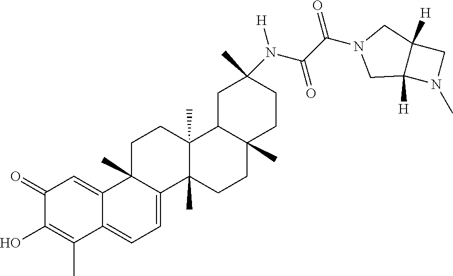 [SMILES: CC1=C(O)C(=O)C=C2C1=CC=C1[C@@]2(C)CC[C@@]2(C)C3C[C@](C)(NC(=O)C(=O)N4C[C@@H]5CN(C)[C@@H]5C4)CC[C@]3(C)CC[C@]12C] | 588 |
| 69 | 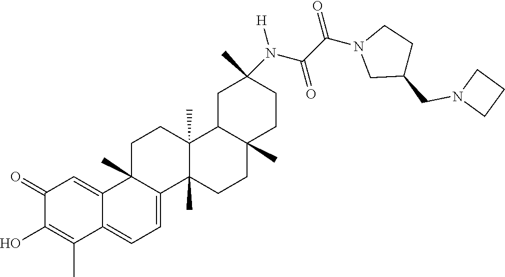 [SMILES: CC1=C(O)C(=O)C=C2C1=CC=C1[C@@]2(C)CC[C@@]2(C)C3C[C@](C)(NC(=O)C(=O)N4CC[C@@H](CN5CCC5)C4)CC[C@]3(C)CC[C@]12C] | 616 |
| 70 | 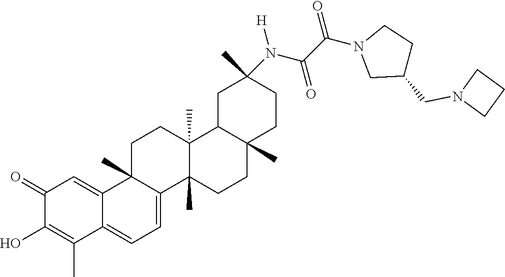 [SMILES: CC1=C(O)C(=O)C=C2C1=CC=C1[C@@]2(C)CC[C@@]2(C)C3C[C@](C)(NC(=O)C(=O)N4CC[C@H](CN5CCC5)C4)CC[C@]3(C)CC[C@]12C] | 616 |
| 71 | 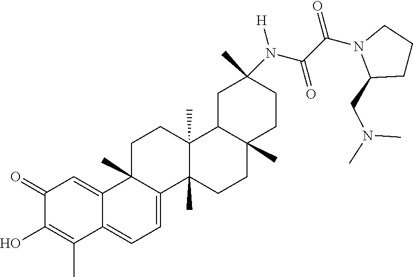 [SMILES: CC1=C(O)C(=O)C=C2C1=CC=C1[C@@]2(C)CC[C@@]2(C)C3C[C@](C)(NC(=O)C(=O)N4CCC[C@H]4CN(C)C)CC[C@]3(C)CC[C@]12C] | 604 |
| 72 | 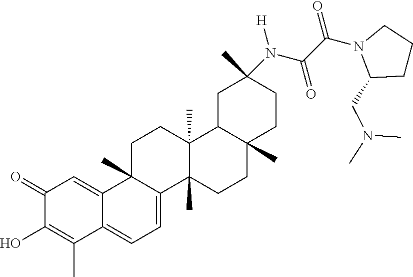 [SMILES: CC1=C(O)C(=O)C=C2C1=CC=C1[C@@]2(C)CC[C@@]2(C)C3C[C@](C)(NC(=O)C(=O)N4CCC[C@@H]4CN(C)C)CC[C@]3(C)CC[C@]12C] | 604 |
| 73 | 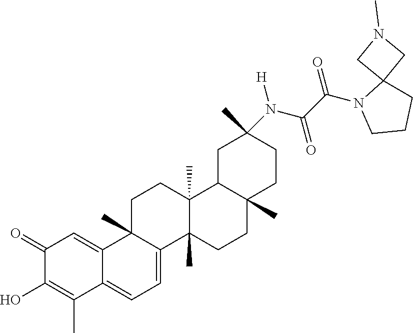 [SMILES: CC1=C(O)C(=O)C=C2C1=CC=C1[C@@]2(C)CC[C@@]2(C)C3C[C@](C)(NC(=O)C(=O)N4CCCC45CN(C)C5)CC[C@]3(C)CC[C@]12C] | 602 |
| 74 | 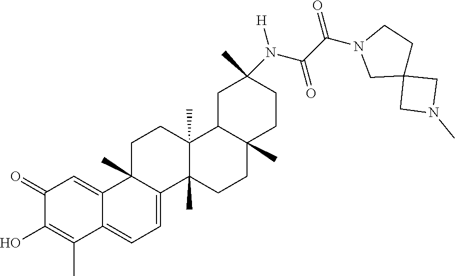 [SMILES: CC1=C(O)C(=O)C=C2C1=CC=C1[C@@]2(C)CC[C@@]2(C)C3C[C@](C)(NC(=O)C(=O)N4CCC5(CN(C)C5)C4)CC[C@]3(C)CC[C@]12C] | 602 |
| 75 | 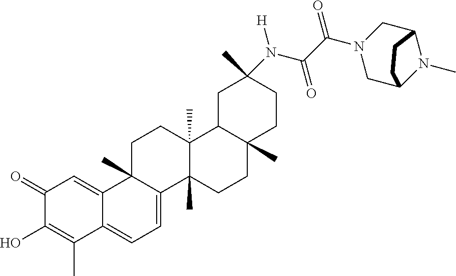 | 602 |
| 76 | 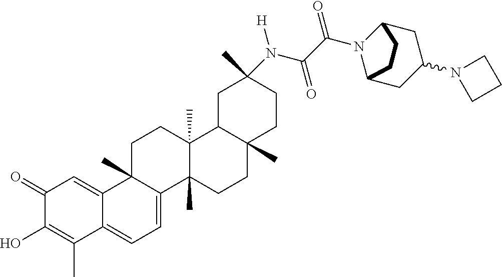 | 642 |
| 77 | 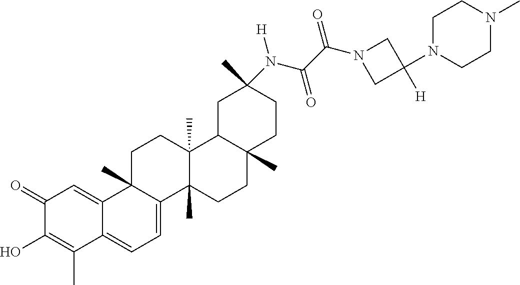 [SMILES: CC1=C(O)C(=O)C=C2C1=CC=C1[C@@]2(C)CC[C@@]2(C)C3C[C@](C)(NC(=O)C(=O)N4CC(N5CCN(C)CC5)C4)CC[C@]3(C)CC[C@]12C] | 631 |
| 78 | 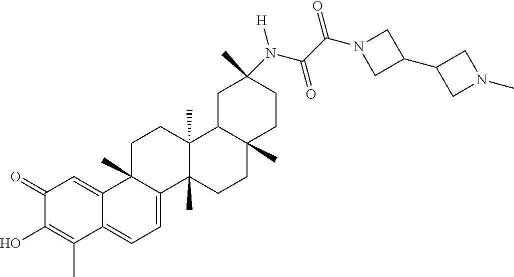 [SMILES: CC1=C(O)C(=O)C=C2C1=CC=C1[C@@]2(C)CC[C@@]2(C)C3C[C@](C)(NC(=O)C(=O)N4CC(C5CN(C)C5)C4)CC[C@]3(C)CC[C@]12C] | 602 |
| 79 | 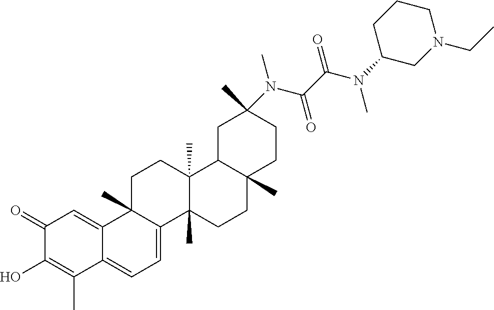 [SMILES: CCN1CCC[C@@H](N(C)C(=O)C(=O)N(C)[C@]2(C)CC[C@]3(C)CC[C@]4(C)C5=CC=C6C(=CC(=O)C(O)=C6C)[C@]5(C)CC[C@@]4(C)C3C2)C1] | 632 |
| 80 | 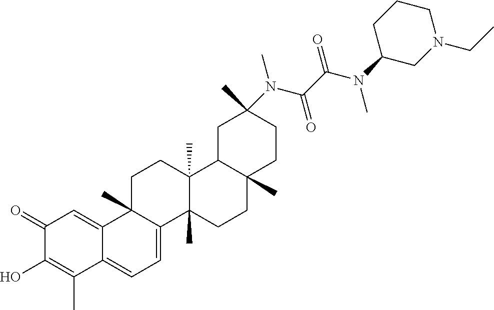 [SMILES: CCN1CCC[C@H](N(C)C(=O)C(=O)N(C)[C@]2(C)CC[C@]3(C)CC[C@]4(C)C5=CC=C6C(=CC(=O)C(O)=C6C)[C@]5(C)CC[C@@]4(C)C3C2)C1] | 632 |
| 81 | 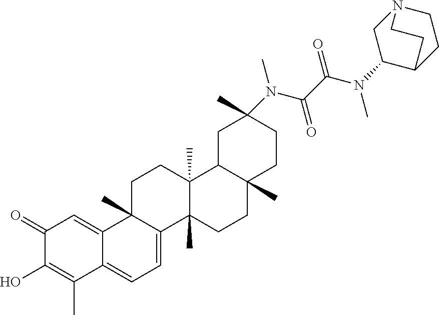 [SMILES: CC1=C(O)C(=O)C=C2C1=CC=C1[C@@]2(C)CC[C@@]2(C)C3C[C@](C)(N(C)C(=O)C(=O)N(C)[C@@H]4CN5CCC4CC5)CC[C@]3(C)CC[C@]12C] | 630 |
| 82 | 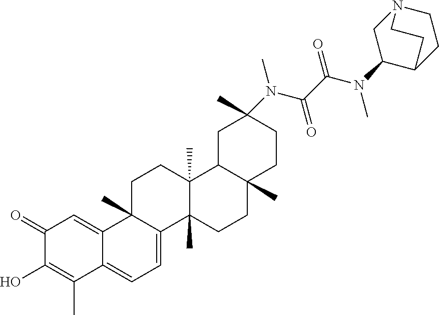 [SMILES: CC1=C(O)C(=O)C=C2C1=CC=C1[C@@]2(C)CC[C@@]2(C)C3C[C@](C)(N(C)C(=O)C(=O)N(C)[C@H]4CN5CCC4CC5)CC[C@]3(C)CC[C@]12C] | 630 |
| 83 | 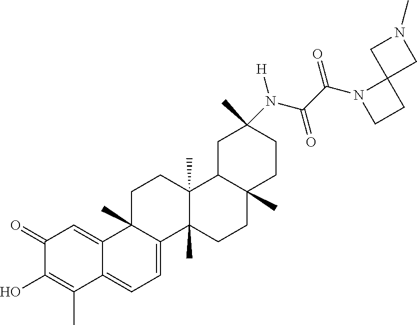 [SMILES: CC1=C(O)C(=O)C=C2C1=CC=C1[C@@]2(C)CC[C@@]2(C)C3C[C@](C)(NC(=O)C(=O)N4CCC45CN(C)C5)CC[C@]3(C)CC[C@]12C] | 588 |
| 84 | 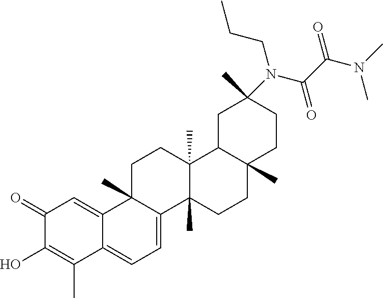 [SMILES: CCCN(C(=O)C(=O)N(C)C)[C@]1(C)CC[C@]2(C)CC[C@]3(C)C4=CC=C5C(=CC(=O)C(O)=C5C)[C@]4(C)CC[C@@]3(C)C2C1] | 563 |
| 85 | 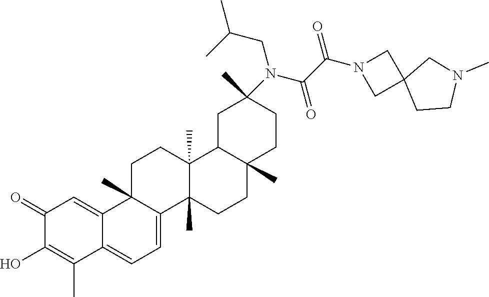 [SMILES: CC1=C(O)C(=O)C=C2C1=CC=C1[C@@]2(C)CC[C@@]2(C)C3C[C@](C)(N(CC(C)C)C(=O)C(=O)N4CC5(CCN(C)C5)C4)CC[C@]3(C)CC[C@]12C] | 658 |
| 86 | 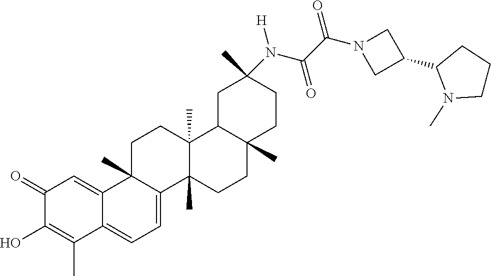 | 616 |
| 87 | 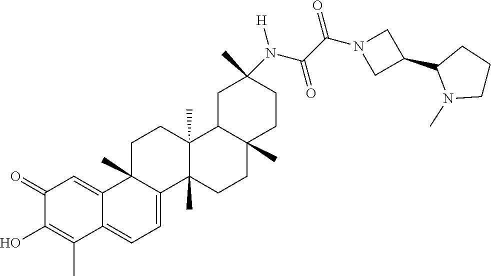 | 616 |
| 88 | 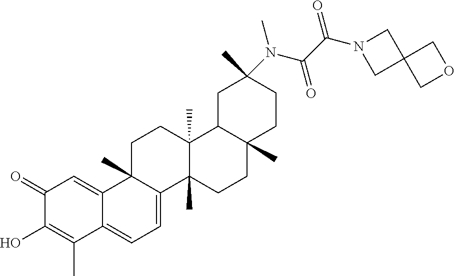 [SMILES: CC1=C(O)C(=O)C=C2C1=CC=C1[C@@]2(C)CC[C@@]2(C)C3C[C@](C)(N(C)C(=O)C(=O)N4CC5(COC5)C4)CC[C@]3(C)CC[C@]12C] | 589 |
| 89 | 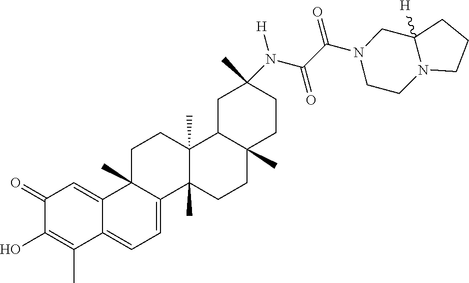 [SMILES: CC1=C(O)C(=O)C=C2C1=CC=C1[C@@]2(C)CC[C@@]2(C)C3C[C@](C)(NC(=O)C(=O)N4CCN5CCCC5C4)CC[C@]3(C)CC[C@]12C] | 602 |
| 90 | 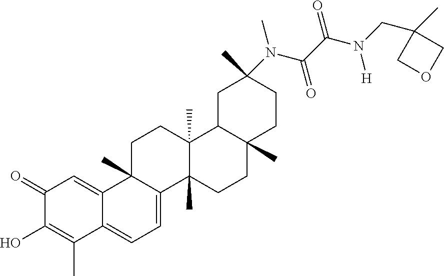 [SMILES: CC1=C(O)C(=O)C=C2C1=CC=C1[C@@]2(C)CC[C@@]2(C)C3C[C@](C)(N(C)C(=O)C(=O)NCC4(C)COC4)CC[C@]3(C)CC[C@]12C] | 591 |
| 91 | 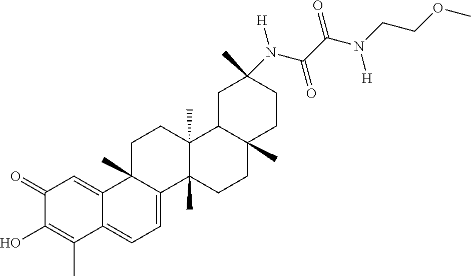 [SMILES: COCCNC(=O)C(=O)N[C@]1(C)CC[C@]2(C)CC[C@]3(C)C4=CC=C5C(=CC(=O)C(O)=C5C)[C@]4(C)CC[C@@]3(C)C2C1] | 551 |
| 92 | 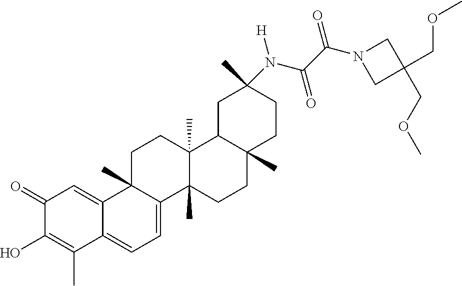 [SMILES: COCC1(COC)CN(C(=O)C(=O)N[C@]2(C)CC[C@]3(C)CC[C@]4(C)C5=CC=C6C(=CC(=O)C(O)=C6C)[C@]5(C)CC[C@@]4(C)C3C2)C1] | 621 |

As described elsewhere herein, one of ordinary skill will understand that the celastrol derivative compounds provided herein can exist in various well-known closely-related and/or equivalent forms not explicitly described by the chemical structures and formulae. It is intended that the celastrol derivative compounds of structural formula (I) of the present disclosure includes these closely-related forms of the compounds defined by the chemical structures and formulae including, but not limited to, pharmaceutically acceptable salts of the compounds, mixture of stereoisomers of the compounds, single stereoisomers of the compounds, tautomeric forms of the compounds, and/or prodrug forms of the compounds.

### III. Preparation of Celastrol Derivative Compounds

The celastrol derivative compounds of structural formula (I) can be prepared from readily available starting materials using methods and procedures known in the art. In particular, the present disclosure provides general synthetic strategies for preparing compounds of structural Formula (I). Schemes A-F presented below provide a series of six reactions starting from celastrol (compound 1) that can be used, adapted, and/or combined with well-known synthetic methods by the ordinary artisan to synthesize the celastrol derivative compounds of the present disclosure, e.g., the compounds of Table 3 (above).

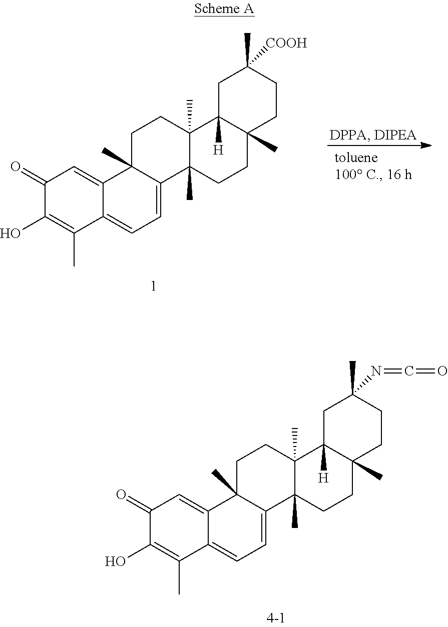

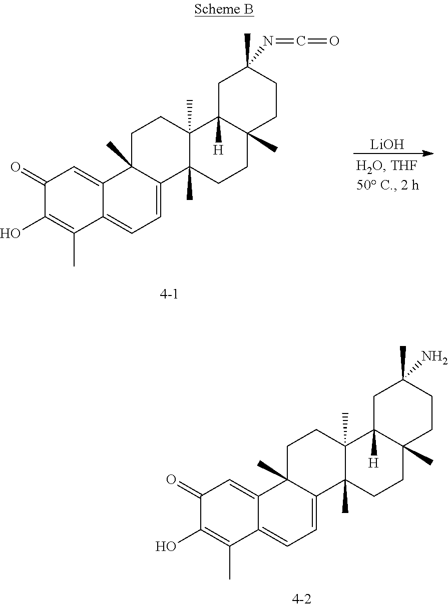

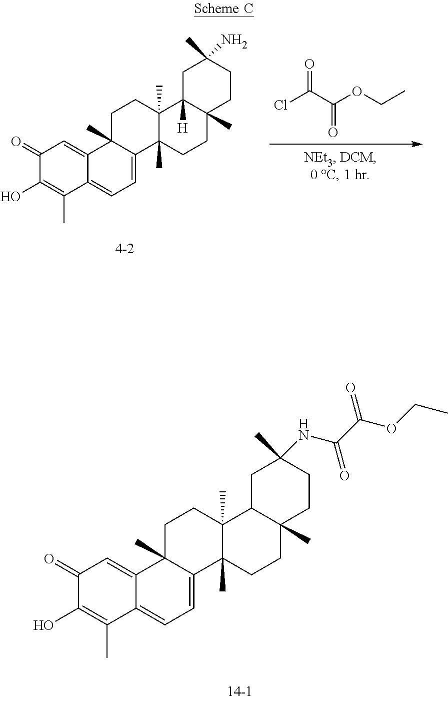

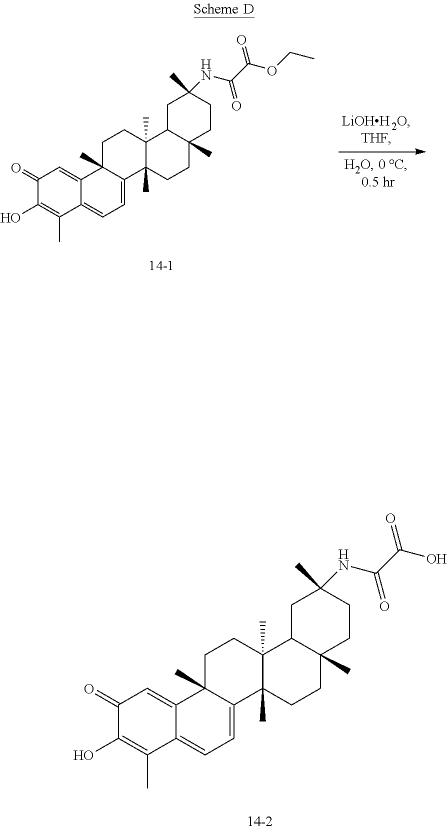

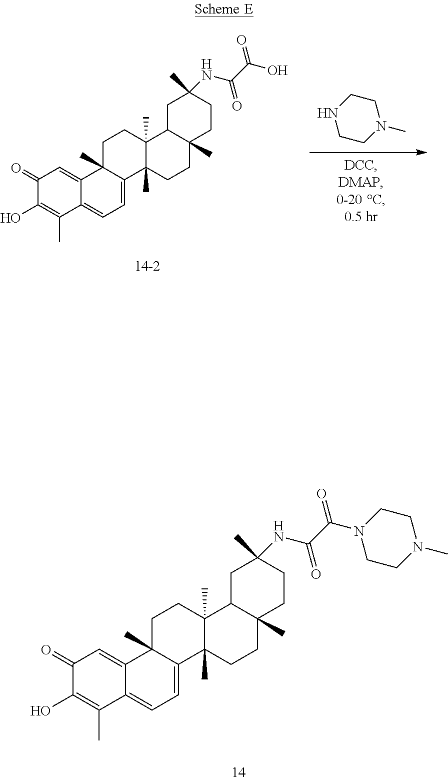

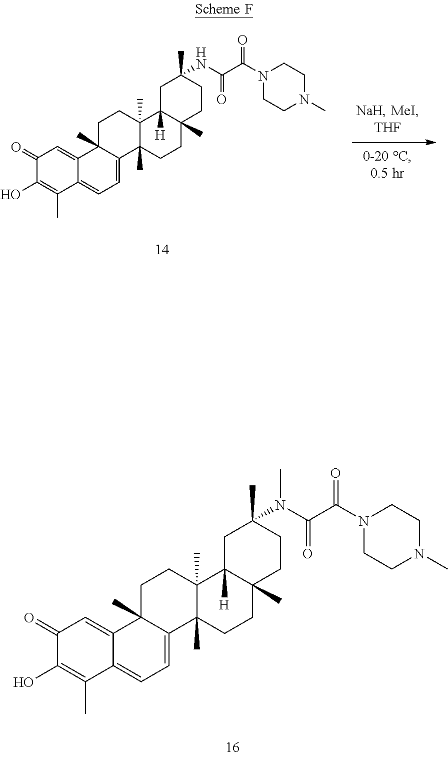

In addition to the reactions of Schemes A-F, Examples 1-7 provide specific synthesis protocols that demonstrate the preparation of the celastrol derivative compounds 14, 15, 16, 17, and 22. One of ordinary skill can use and/or adapt these synthesis protocols of Examples 1-7 for the preparation of additional celastrol derivative compounds of structural formula (I) described in the present disclosure. It will be appreciated by one of ordinary skill that in addition to the typical or preferred process conditions (i.e., reaction temperatures, times, mole ratios of reactants, solvents, pressures, etc.) described in the Examples herein, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be necessary to prevent certain functional groups from undergoing undesired reactions. For example, a protecting group may be used to allow a functional group (such as O, S, or N) to be temporarily blocked so that a reaction can be carried out selectively at another reactive site in a multifunctional compound. Protecting groups useful in syntheses of the present disclosure are well known in the art and include those described in detail in Protective Groups in Organic Synthesis, Fourth Ed., Greene, T. W. and Wuts, P. G., Eds., John Wiley & Sons, New York: 2007, the entire contents of which are hereby incorporated by reference, and references cited therein.

As noted above, the starting materials and/or reagents used in the synthetic reaction Schemes A-F are commercially available, or generally known compounds that can be prepared by known procedures or obvious modifications thereof or are disclosed in the Examples herein. For example, many of the materials/reagents are available from commercial suppliers such as Sigma-Aldrich Chemical Co. (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### IV. Uses and Methods of Treatments

As described elsewhere herein, celastrol has been proposed or shown to provide a potentially therapeutic effect, based on clinical, pre-clinical, or in vitro studies, in a number of diseases and disorders. These previously identified celastrol-responsive diseases and disorders include, but are not limited to, cancer (e.g., gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme), inflammatory and/or autoimmune disorders (e.g., rheumatoid arthritis (RA), ankylosing spondylitis, systemic lupus erythematosus (SLE), inflammatory bowel disease, osteoarthritis (OA), allergy, and skin inflammation), neurological disorders (e.g., Parkinson's disease, Huntington disease, Alzheimer's disease, and amyotrophic lateral sclerosis (ALS)), obesity-related disorders (e.g., obesity, pre-obesity, morbid obesity, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome and MOMO (Macrosomia Obesity Macrocephaly Ocular abnormalities) Syndrome), and liver-related diseases or disorders (e.g., acute-chronic liver failure (ACLF), alcoholic liver disease, cholestatic liver disease, drug-induced liver disease, hepatocellular carcinoma, non-alcoholic fatty liver disease (NAFLD), non-alcoholic steatohepatitis (NASH), viral hepatitis, and viral liver disease).

Accordingly, the present disclosure contemplates the use of a celastrol derivative compound of structural formula (I) of the present disclosure, or a pharmaceutical composition of such a compound, in a therapy, as a medicament, or in a method of treating a celastrol-responsive disease or disorder in a subject, or in the manufacture of a medicament for treating a celastrol-responsive disease or disorder in a subject.

In at least one embodiment, the present disclosure contemplates that the celastrol derivative compounds of structural formula (I) disclosed herein can be used in methods of treating a celastrol-responsive disease or disorder in a subject suffering therefrom. Generally, the method of treatment comprises administering to the subject in need thereof, a therapeutically effective amount of a compound of structural formula (I) as disclosed herein. In at least one embodiment, the administered compound of structure formula (I) can be a compound is selected from compound 14, compound 15, compound 16, compound 17, compound 22, compound 22-1, compound 42, compound 54, compound 55, compound 56, compound 57, compound 58, compound 59, compound 60, compound 61, compound 62, compound 63, compound 64, compound 65, compound 66, compound 67, compound 68, compound 69, compound 70, compound 71, compound 72, compound 73, compound 74, compound 75, compound 76, compound 77, compound 78, compound 79, compound 80, compound 81, compound 82, compound 83, compound 84, compound 85, compound 86, compound 87, compound 88, compound 89, compound 90, compound 91, and compound 92, or a pharmaceutically acceptable salt, single stereoisomer, mixture of stereoisomers, tautomer, or prodrug of any one of these compounds.

In at least one embodiment, the administered compound of structure formula (I) can be in the form of a pharmaceutical composition comprising the compound of structural formula (I) or a pharmaceutically acceptable salt or ester thereof, and one or more pharmaceutically acceptable excipients, as described elsewhere herein. In at least one embodiment, the administered pharmaceutical composition can comprise one or more pharmaceutically acceptable excipients and a compound is selected from compound 14, compound 15, compound 16, compound 17, compound 22, compound 22-1, compound 42, compound 54, compound 55, compound 56, compound 57, compound 58, compound 59, compound 60, compound 61, compound 62, compound 63, compound 64, compound 65, compound 66, compound 67, compound 68, compound 69, compound 70, compound 71, compound 72, compound 73, compound 74, compound 75, compound 76, compound 77, compound 78, compound 79, compound 80, compound 81, compound 82, compound 83, compound 84, compound 85, compound 86, compound 87, compound 88, compound 89, compound 90, compound 91, and compound 92, or a pharmaceutically acceptable salt, single stereoisomer, mixture of stereoisomers, tautomer, or prodrug of any one of these compounds.

In at least one embodiment of the use or therapeutic method of treatment of the celastrol derivative compound of structural formula (I), the celastrol-responsive disease or disorder that the subject is suffering from can be a cancer. The cancer treated by the method can include, but is not limited to, gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme (GBM).

In at least one embodiment of the use or therapeutic method of treatment of the celastrol derivative compound of structural formula (I), the celastrol-responsive disease or disorder that the subject is suffering from can be an inflammatory and/or autoimmune disorder. The inflammatory and/or autoimmune disorder treated by the method can include, but is not limited to, rheumatoid arthritis (RA), ankylosing spondylitis, systemic lupus erythematosus (SLE), inflammatory bowel disease, osteoarthritis (OA), allergy, and skin inflammation.

In at least one embodiment of the use or therapeutic method of treatment of the celastrol derivative compound of structural formula (I), the celastrol-responsive disease or disorder that the subject is suffering from can be a neurological disorder. The neurological disorder treated by the method can include, but is not limited to, Parkinson's disease, Huntington disease, Alzheimer's disease, and amyotrophic lateral sclerosis (ALS).

In at least one embodiment of the use or therapeutic method of treatment of the celastrol derivative compound of structural formula (I), the celastrol-responsive disease or disorder that the subject is suffering from can be an obesity-related disease or disorder. The obesity-related disease or disorder treated by the method can include, but is not limited to, obesity, pre-obesity, morbid obesity, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome, and MOMO (Macrosomia Obesity Macrocephaly Ocular abnormalities) Syndrome.

In at least one embodiment of the use or therapeutic method of treatment of the celastrol derivative compound of structural formula (I), the celastrol-responsive disease or disorder that the subject is suffering from can be a liver-related disease or disorder. The liver-related disease or disorder treated by the method can include, but is not limited to, acute-chronic liver failure (ACLF), acute hepatic porphyria, Alagille syndrome, alpha-1 antitrypsin deficiency, alcoholic liver disease, alcoholic hepatitis, amoebic liver, autoimmune hepatitis, benign liver tumors, biliary atresia, cardiac cirrhosis, cholestatic liver disease, congestive hepatopathy, cirrhosis, Crigler-Najjar syndrome, drug-induced liver disease, Dubin-Johnson syndrome, conjugated hyperbilirubinemia, galactosemia, Gilbert syndrome, glycogen storage disease, hemochromatosis, hepatic abscesses, hepatic cysts, hepatic encephalopathy, hepatitis, hepatorenal syndrome, intrahepatic cholestasis of pregnancy, isoniazid toxicity, jaundice, liver abscess, liver cyst, liver cancer, liver disease in pregnancy, lysosomal acid lipase deficiency (LAL-D), neonatal jaundice, non-alcoholic fatty liver disease (NAFLD), non-alcoholic steatohepatitis (NASH), primary biliary cholangitis, primary sclerosing cholangitis, progressive familial intrahepatic cholestasis, Reye syndrome, Type I glycogen storage disease, unconjugated hyperbilirubinemia, viral hepatitis, viral liver disease, and Wilson's disease. In at least one embodiment, the liver-related disease or disorder treated by the method is selected from acute-chronic liver failure (ACLF), alcoholic liver disease, cholestatic liver disease, drug-induced liver disease, hepatocellular carcinoma, non-alcoholic fatty liver disease (NAFLD), non-alcoholic steatohepatitis (NASH), viral hepatitis, and viral liver disease.

### V. Pharmaceutical Compositions and Modes of Administering

The present disclosure also provides uses and methods in which a celastrol derivative compound, such as a compound of structural formula (I), is administered to a subject in the form of a pharmaceutical composition, as described above. For example, in such embodiments, the pharmaceutical composition includes a therapeutically effective amount of the celastrol derivative compound of structural formula (I) (e.g., a compound of Table 3), or a pharmaceutically acceptable salt or ester such a compound and one or more pharmaceutically acceptable carrier. Such pharmaceutical compositions can be prepared using methods well known in the pharmaceutical art (see, e.g., Remington's Pharmaceutical Sciences, Mace Publishing Co., Philadelphia, PA 17th Ed. (1985) and Modern Pharmaceutics, Marcel Dekker, Inc. 3rd Ed. (G. S. Banker & C. T. Rhodes, Eds.). Methods of preparing pharmaceutical compositions of celastrol derivative compounds are described in the present disclosure, including the Examples disclosed herein.

Generally, the pharmaceutical compositions can be prepared by diluting the active ingredient(s) with an excipient and/or enclosing it within a carrier in the form of a capsule, sachet, paper, or other container. When the excipient serves as a diluent, it can be a solid, semi-solid, or liquid material (as above), which acts as a vehicle, carrier or medium for the active ingredient. Thus, the pharmaceutical composition(s) suitable for administering in the methods of the disclosure can be in the dosage form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

The carriers used in the preparation of the pharmaceutical compositions can include excipients such as inert solid diluents and fillers, diluents, including sterile aqueous solution and various organic solvents, permeation enhancers, solubilizers and adjuvants. Suitable excipients for use in the pharmaceutical compositions comprising a celastrol derivative of the present disclosure (e.g., compounds of Table 3) are well known in the art and include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include lubricating agents such as talc, magnesium stearate, and mineral oil; wetting agents; emulsifying and suspending agents; preserving agents such as methyl- and propylhydroxy-benzoates; sweetening agents; and flavoring agents.

In the uses and methods of treatment, it is contemplated that the pharmaceutical composition comprising the celastrol derivative compounds, such as a compound of structural Formula (I) (e.g., compounds of Table 3), can be administered either as single or multiple doses, and by any of the accepted modes of administration of active ingredients having similar utility. For example, a pharmaceutical composition comprising an celastrol derivative can be administered using a variety of different modes including oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The pharmaceutical compositions including the celastrol derivative compounds of the present disclosure (e.g., compounds of Table 3) can be used in a range of therapeutic methods of treatment and a range of dosages are contemplated for administration of a pharmaceutically effective amount. The dosage and frequency (single or multiple doses) of administration of the pharmaceutical composition to a subject can vary depending upon a range of factors, such as, the route of administration; the subject's size, age, sex, health, body mass, and/or diet; the state of the disease being treated; whether the subject is suffering from any other diseases, and any concurrent treatment being received. One of ordinary skill will understand that adjustment of established dosages (e.g., frequency and duration) to obtain the therapeutically effective amount may be required depending on the subject. Typically, the amount of a pharmaceutical composition containing a celastrol derivative compound to be administered to a subject in a therapeutic method of treatment will be determined by a physician, in view of relevant circumstances of the subject being so treated, the chosen route of administration, and of course, the age, the weight, the severity of symptoms, the response of the individual subject to the treatment, and the like.

Generally, a therapeutically effective amount is the amount sufficient for the administered composition to accomplish a desired therapeutic purpose relative to the absence of the compound. For example, the therapeutically effective amount can be the amount determined to be sufficient to contribute to the treatment, prevention, or reduction of a symptom or symptoms of a disease. Methods for determining the dosage providing a therapeutically effective amount of a compound are well-known to those of ordinary skill in the art, and typically are based on analysis of amounts determined in cellular assays and/or animal models.

For example, a dosage for administration to humans can be formulated to achieve a concentration that has been observed as therapeutically effective in an animal model. The dosage in the pharmaceutical composition for humans can further be adjusted by monitoring the effectiveness and adjusting upwards or downwards. One of ordinary skill can used methods well known in the art to adjust the dosage in a pharmaceutical composition of the present disclosure (e.g., compounds of Table 3) to achieve maximal therapeutic efficacy for humans.

Generally, methods for therapeutic treatment are developed by starting with a pharmaceutical composition containing less than the optimal dose of the celastrol derivative compound. Thereafter, the dosage of the compound is increased incrementally until optimal efficacy is attained. A key factor considered in developing the optimal dose is the ratio between the toxicity and the therapeutic efficacy of the active ingredient. This ratio, referred to as the compound's therapeutic index, is typically described as the ratio of the active ingredient's LD₅₀ (the amount of compound lethal in 50% of the population) to its ED₅₀ (the amount of compound effective in 50% of the population). Typically, a higher therapeutic index for a compound is preferred. Therapeutic index data can be obtained from cell culture assays and/or animal model studies and then used to determine a safe range of dosages of the active ingredient in a pharmaceutical composition for administration to humans. Ideally the dosage determined provides the active ingredient at its ED₅₀ level in the subject with little or no toxicity.

In some embodiments using the compounds of the present disclosure (e.g., compounds of Table 3), the pharmaceutical composition contains a dosage of the celastrol derivative compound as the active ingredient in an amount of about 0.05 to about 100 mg/kg, about 0.1 to about 0.5 mg/kg, about 0.1 to about 1 mg/kg, about 0.1 to about 5 mg/kg, about 0.1 to about 10 mg/kg, about 0.1 to about 25 mg/kg, about 1 to about 5 mg/kg, about 1 to about 25 mg/kg, about 5 to about 25 mg/kg, about 10 to about 25 mg/kg, about 10 to about 50 mg/kg, about 25 to about 50 mg/kg, about 25 to about 75 mg/kg, or about 50 to about 100 mg/kg. In some embodiments, the pharmaceutical composition comprises a dosage of the celastrol derivative compound of Formula (I) in an amount of about 0.05 mg/kg, about 0.1 mg/kg, about 1 mg/kg, about 5 mg/kg, about 10 mg/kg, about 25 mg/kg, about 50 mg/kg, about 75 mg/kg, or about 100 mg/kg.

In some embodiments it is contemplated that the pharmaceutical compositions comprising celastrol derivative compounds of the present disclosure (e.g., compounds of Table 3) can be formulated so as to provide quick, sustained, or delayed release of the active ingredient after administration. For example, controlled release drug delivery systems for oral administration are known in the art and include osmotic pump systems and dissolutional systems containing polymer-coated reservoirs or drug-polymer matrix formulations. Examples of controlled release systems are given in e.g., U.S. Pat. Nos. 3,845,770; 4,326,525; 4,902,514; and 5,616,345.

In some embodiments, it is contemplated that the pharmaceutical compositions comprising a celastrol derivative compound of the present disclosure (e.g., compounds of Table 3) can also be formulated for administration via transdermal delivery devices (e.g., “patches”). Such transdermal patches may be used to provide continuous or discontinuous infusion of the pharmaceutical compositions in controlled amounts. The construction and use of transdermal patches for the delivery of pharmaceutical compositions is well known in the art. See, e.g., U.S. Pat. Nos. 5,023,252, 4,992,445 and 5,001,139. Such patches may be constructed for continuous, pulsatile, or on demand delivery of the pharmaceutical composition(s).

In some embodiments, it is contemplated that the pharmaceutical composition of the present disclosure (e.g., compounds of Table 3) can be prepared as a solid formulation, e.g., for oral administration. Such solid formulations can be prepared by mixing the celastrol derivative compound active ingredient with a pharmaceutical excipient to form a solid pre-formulation composition containing a homogeneous mixture of the active ingredient and the excipients. When referring to these pre-formulation compositions as homogeneous, it is meant that the active ingredient is dispersed evenly throughout the composition so that the composition may be readily subdivided into equally effective unit dosage forms such as tablets, pills, and capsules. Tablets or pills may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Another exemplary mode for administering useful in the methods of the present disclosure is parenteral, particularly by injection. Pharmaceutical compositions of the present disclosure (e.g., compositions including compounds of Table 3) may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the active ingredients of the present disclosure (e.g., compounds of Table 3) in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the known methods of preparation include vacuum-drying and freeze-drying techniques which yield a powder of the active ingredient plus any additional desired ingredient from a previously sterile-filtered solution thereof.

Pharmaceutical compositions that can be administered by inhalation or insufflation include solutions and suspensions in pharmaceutically acceptable, aqueous organic solvents, or mixtures thereof, and powders. The liquid or solid compositions may contain suitable pharmaceutically acceptable excipients as described herein and as known in the art. In some embodiments, the pharmaceutical composition of the celastrol derivative (e.g., compound of Table 3) can be administered by the oral or nasal respiratory route for local or systemic effect. In some embodiments, the pharmaceutical compositions are prepared in pharmaceutically acceptable solvents which can be nebulized by use of inert gases. These nebulized solutions can be inhaled directly from the nebulizing device or the nebulizing device may be attached to a face mask tent, or intermittent positive pressure breathing machine. In some embodiments, the pharmaceutical compositions useful in the methods can be in solution, suspension, or powder compositions and can be administered, orally or nasally, from devices that deliver the formulation in an appropriate manner.

### EXAMPLES

Various features and embodiments of the disclosure are illustrated in the following representative examples, which are intended to be illustrative, and not limiting. Those skilled in the art will readily appreciate that the specific examples are only illustrative of the various embodiments of the present disclosure as described more fully in the claims which follow thereafter. Every embodiment and feature described in the application should be understood to be interchangeable and combinable with every embodiment contained within.

### Example 1: Synthesis of (6aS,6bS,8aS,11R,12aR,14aR)-11-amino-3-hydroxy-4,6a,6b,8a,11,14a-hexamethyl-7,8,9,10,12,12a,13,14-octahydropicen-2-one (Compound 4-2)

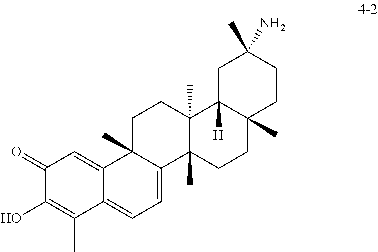

Compound 4-2 was prepared via the 2-step synthesis summarized in Scheme 1 (below).

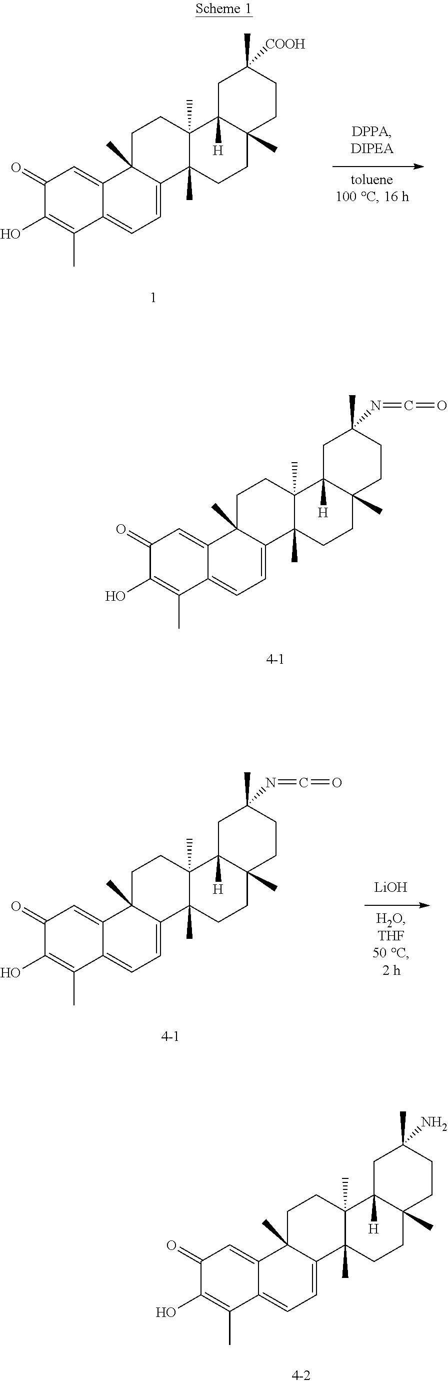

### Step 1

### Synthesis of (6aS,6bS,8aS,11R,12aR,14aR)-3-hydroxy-11-isocyanato-4,6a,6b,8a,11,14a-hexamethyl-7,8,9,10,12,12a,13,14-octahydropicen-2-one (Compound 4-1)

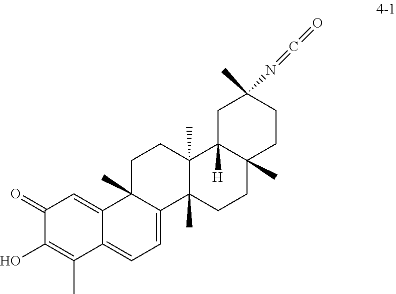

To a solution of (2R,4aS,6aR,6aS,14aS,14bR)-10-hydroxy-2,4a,6a,6a,9,14a-hexamethyl-11-oxo-1,3,4,5,6,13,14,14b-octahydropicene-2-carboxylic acid (4.0 g, 8.88 mmol) in toluene (200 mL) was added DIPEA (3.44 g, 26.6 mmol) and DPPA (3.66 g, 13.3 mmol) at 25° C. under N₂. The reaction mixture was stirred at 100° C. for 16 hours. The reaction mixture was cooled to 25° C. and poured into water (200 mL). The aqueous phase was extracted with DCM (3×100 mL). The combined organic phase was washed with brine (2×300 mL), dried overanhydrous Na₂SO₄, filtered and concentrated. The residue was purified by silica gel chromatography (Eluent of 0~50% ethyl acetate/petroleum ether) to afford (6aS,6bS,8aS,11R,12aR,14aR)-3-hydroxy-11-isocyanato-4,6a,6b,8a,11,14a-hexamethyl-7,8,9,10,12,12a,13,14-octahydropicen-2-one (compound 4-1) (3.0 g, 91.4% purity, 76% yield) and (142 mg, 100% purity) both as yellow solid.

¹H NMR: CDCl₃ 400 MHz δ=7.05 (d, J=6.8 Hz, 1H), 6.56 (s, 1H), 6.38 (d, J=7.2 Hz, 1H), 2.24-1.90 (m, 8H), 1.85-1.52 (m, 10H), 1.46 (s, 3H), 1.42 (s, 3H), 1.28 (s, 3H), 1.10-0.99 (m, 4H), 0.95 (s, 3H)

LCMS: Rₜ=2.949 min in 1.5 min chromatography, 5-95AB, purity 99%, LCMS ESI+ calcd, for C₂₉H₃₇NO₃ [M]⁺ 447.2, found [M+H]⁺ 448.2.

HPLC: Rₜ=4.74 min in 8 min chromatography, 50-100AB, purity 96%.

### Step 2

### Synthesis of (6aS,6bS,8aS,11R,12aR,14aR)-11-amino-3-hydroxy-4,6a,6b,8a,11,14a-hexamethyl-7,8,9,10,12,12a,13,14-octahydropicen-2-one (Compound 4-2)

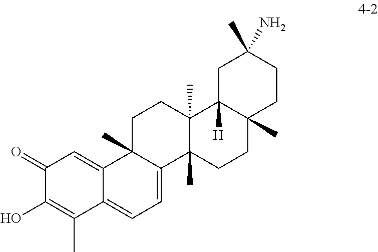

A solution of (6aS,6bS,8aS,11R,12aR,14aR)-3-hydroxy-11-isocyanato-4,6a,6b,8a,11,14a-hexamethyl-7,8,9,10,12,12a,13,14-octahydropicen-2-one (3.0 g, 6.70 mmol) in THF (120 mL) and H₂O (12 mL) was added LiOH—H₂O (1.41 g, 33.5 mmol) at 25° C. The reaction mixture was stirred at 50° C. for 2 hours. The reaction mixture was concentrated and then poured into H₂O (200 mL) and filtered to give a solid. The solid was dissolved in MeOH and concentrated in vacuum to give (6aS,6bS,8aS,11R,12aR,14aR)-11-amino-3-hydroxy-4,6a,6b,8a,11,14a-hexamethyl-7,8,9,10,12,12a,13,14-octahydropicen-2-one (3.5 g, crude) as yellow solid. The crude product (50 mg, crude) was purified by silica gel chromatography (eluent of 0~20% methanol/dichloromethane) to give (6aS,6bS,8aS,11R,12aR,14aR)-11-amino-3-hydroxy-4,6a,6b,8a,11,14a-hexamethyl-7,8,9,10,12,12a,13,14-octahydropicen-2-one (compound 4-2) (11.3 mg, 100% purity) as yellow solid.

¹H NMR: MeOD 400 MHz δ=7.22 (d, J=7.6 Hz, 1H), 6.52 (d, J=7.2 Hz, 1H), 6.47 (s, 1H), 2.34-2.15 (m, 4H), 2.05-1.61 (m, 14H), 1.61-1.13 (m, 15H), 0.85 (s, 3H).

LCMS: Rₜ=0.920 min in 1.5 min chromatography, 5-95AB, purity 100%, LCMS ESI+ calcd, for C₂₈H₃₉NO₂ [M]⁺ 421.3, found [M+H]⁺ 422.2.

HPLC: Rₜ=2.56 min in 8 min chromatography, 30-90AB, purity 100%.

### Example 2: Synthesis of (ethyl 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetate (Compound 14-1)

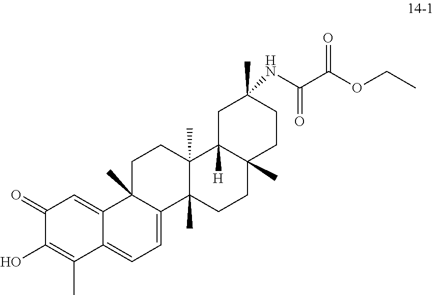

Compound 14-1 was prepared via synthesis summarized in Scheme 2 (below).

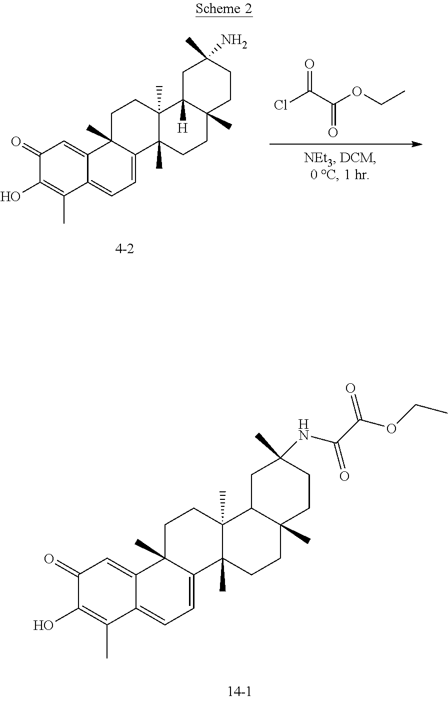

### Step 1

To a solution of (6bS,8aS,11R,12aR,12bS,14aR)-11-amino-3-hydroxy-4,6b,8a,11,12b,14a-hexamethyl-7,8,8a,9,10,11,12,12a,12b,13,14,14a-dodecahydropicen-2(6bH)-one (1.00 g, 2.37 mmol) in DCM (20 mL) was added Et₃N (720 mg, 7.12 mmol) at 0° C. Then ethyl 2-chloro-2-oxo-acetate (291 mg, 2.13 mmol) was added dropwise. The resulting mixture was stirred at 0° C. for 1 hour. The reaction mixture was poured into water (50 mL). The aqueous phase was extracted with DCM (3×100 mL). The combined organic phase was washed with brine (3×30 mL), dried over anhydrous Na₂SO₄, filtered and concentrated to give ethyl 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetate (1.2 g, 74.7% purity) as yellow solid. 100 mg of the yellow solid was purified by prep-HPLC (column: Phenomenex Gemini 150*25 mm*10 μm; mobile phase: [water (0.05% NH₃H₂O)-ACN]; B %: 62%-92%, 10 min) to give ethyl 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetate (compound 14-1) (15.25 mg, 96.7% purity) as a yellow solid.

1H NMR: CDCl3 400 MHz δ=7.08-6.78 (m, 2H), 6.63-6.26 (m, 2H), 4.42 (dd, J=4.5, 8.0 Hz, 1H), 4.39-3.95 (m, 2H), 2.66 (br d, J=15.0 Hz, 1H), 2.32-2.11 (m, 4H), 2.02-1.52 (m, 15H), 1.39-1.28 (m, 6H), 1.22-1.00 (m, 5H), 0.87 (br d, J=7.0 Hz, 2H), 0.72 (br s, 3H).

LCMS: Rt=0.595 min in 1.5 min chromatography, 50-95AB, purity 100%, LCMS ESI+ calcd, for C₃₂H₄₃NO₅ [M]+ 521.3, found [M+H]+ 522.3.

HPLC: Rt=3.101 min in 4 min chromatography, 10-80AB, purity 96.73%.

### Example 3: Synthesis of N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-(4-methylpiperazin-1-yl)-2-oxoacetamide (Compound 14)

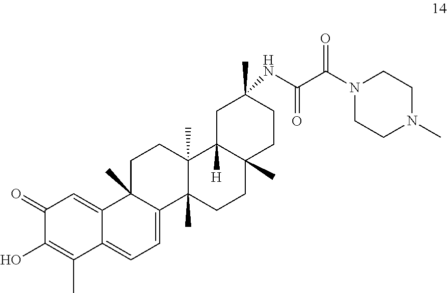

Compound 14 was prepared via the 2-step synthesis summarized in Scheme 3 (below).

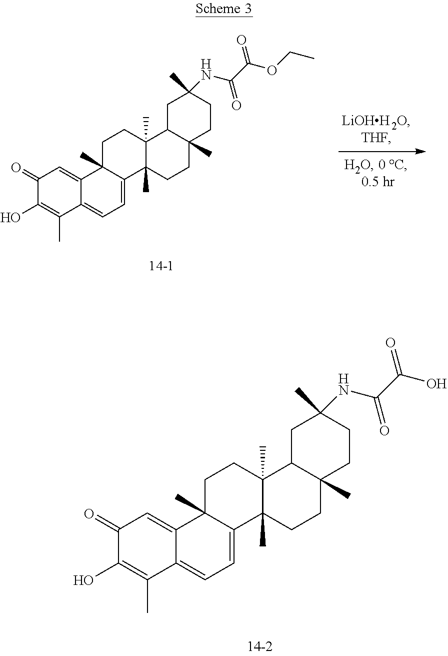

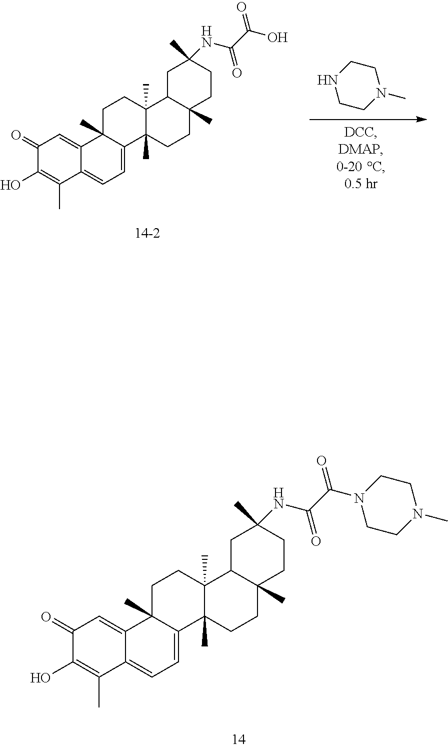

### Step 1

### Synthesis of 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetic acid (Compound 14-2)

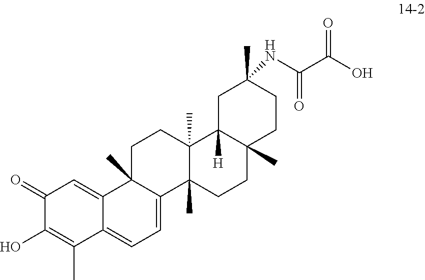

A mixture of ethyl 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetate (1.3 g, 2.49 mmol) and LiOH·H₂O (314 mg, 7.48 mmol) in THF (60 mL) and H₂O (12 mL) was stirred at 0° C. for 0.5 hour. The reaction mixture was adjusted to pH=3 by adding aq. HCl (1 M). The aqueous phase was extracted with DCM (30 mL). The combined organic phase was dried over anhydrous Na₂SO₄, filtered and concentrated under reduce pressure to give 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetic acid (compound 14-2) (1.2 g, 80.2% yield) as a yellow solid.

LCMS: Rt=0.507 min in 1.5 min chromatography, 5-95AB, purity 73.919%, LCMS ESI+ calcd, for C₃₀H₃₉NO₅ [M]+ 493.3, found [M+H]+ 494.3.

### Step 2

### Synthesis of N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-(4-methylpiperazin-1-yl)-2-oxoacetamide (Compound 14)

To a solution of 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetic acid (500 mg, 1.01 mmol) in DCM (5 mL) was added DCC (313 mg, 1.52 mmol) and stirred at 0° C. 1-methylpiperazine (152 mg, 1.52 mmol) and DMAP (61.9 mg, 506 μmol) was added at 0° C. The resulting mixture was stirred at 20° C. for 0.5 hour. The reaction mixture was poured into water (5 mL). The aqueous phase was extracted with DCM (3×10 mL). The combined organic phase was dried over anhydrous Na₂SO₄, filtered, and concentrated under reduced pressure. The residue was purified by column chromatography (dichloromethane:methanol=1/0 to 20/1 Rf=0.32) to give N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-(4-methylpiperazin-1-yl)-2-oxoacetamide (200 mg) as a brown solid. The brown solid was purified by prep-TLC (SiO₂, Dichloromethane:Methanol=10/1, Rf=0.32) to give N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-(4-methylpiperazin-1-yl)-2-oxoacetamide (Compound 14) (100 mg, 15.3% yield) as a yellow solid.

1H NMR: CDCl3 400 MHz δ=7.09-6.90 (m, 3H), 6.51 (d, J=1.4 Hz, 1H), 6.36 (d, J=7.1 Hz, 1H), 4.43-4.00 (m, 2H), 3.87-3.45 (m, 2H), 2.72 (br d, J=14.0 Hz, 1H), 2.64-2.39 (m, 4H), 2.31 (s, 3H), 2.24-2.11 (m, 4H), 2.06-1.67 (m, 11H), 1.51-1.50 (m, 1H), 1.45 (d, J=6.8 Hz, 6H), 1.34-1.23 (m, 4H), 1.13 (s, 3H), 1.06-0.98 (m, 1H), 0.75 (s, 3H).

LCMS: Rt=0.431 min in 1.5 min chromatography, 5-95AB, purity 98.137%, LCMS ESI+ calcd, for C₃₅H₄₉N₃O₄ [M]+ 575.4, found [M+H]+ 576.4.

HPLC: Rt=2.105 min in 4 min chromatography, 10-80AB, purity 95.8%.

### Example 4: Synthesis of N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-oxo-2-(2-oxa-6-azaspiro[3.3]heptan-6-yl)acetamide (Compound 15)

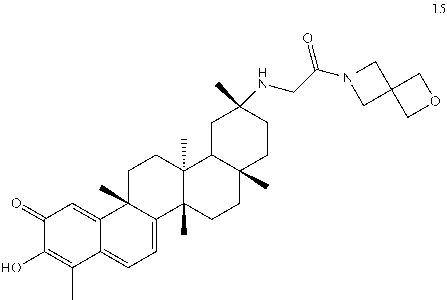 [SMILES: CC1=C(O)C(=O)C=C2C1=CC=C1[C@@]2(C)CC[C@@]2(C)C3C[C@](C)(NCC(=O)N4CC5(COC5)C4)CC[C@]3(C)CC[C@]12C]

Compound 15 was prepared via the synthesis summarized in Scheme 4 (below).

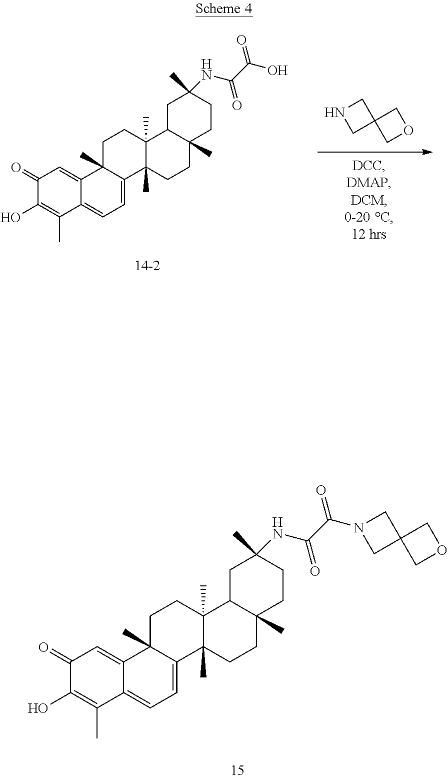

### Step 1

To a solution of 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetic acid (compound 14-2) (100 mg, 203 μmol) in DCM (1.5 mL) was added DCC (105 mg, 506 μmol) and stirred at 0° C. Then 2-oxa-6-azaspiro[3.3]heptane (22.1 mg, 223 μmol) and DMAP (37.1 mg, 304 μmol) was added. The resulting mixture was stirred at 20° C. for 12 hours. The reaction mixture was poured into water (5 mL). The aqueous phase was extracted with DCM (3×10 mL). The combined organic phase was dried over anhydrous Na₂SO₄, filtered, and concentrated under reduced pressure. The residue was purified by column chromatography (petroleum ether:ethyl acetate=1/0 to 0/1 Rf=0.26) to give a crude brown solid (60 mg). The brown solid was purified by prep-TLC to give N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-oxo-2-(2-oxa-6-azaspiro[3.3]heptan-6-yl)acetamide (compound 15) (5.5 mg, 3.11% yield) as yellow solid.

¹H NMR: CDCl₃ 400 MHz δ=7.21 (s, 1H), 7.04 (br d, J=7.0 Hz, 2H), 6.55 (s, 1H), 6.36 (d, J=7.1 Hz, 1H), 4.90-4.68 (m, 7H), 4.19 (s, 2H), 2.63 (br d, J=14.4 Hz, 1H), 2.27-2.10 (m, 4H), 2.04-1.83 (m, 4H), 1.71 (br d, J=8.4 Hz, 4H), 1.64-1.58 (m, 3H), 1.48-1.40 (m, 6H), 1.28 (s, 3H), 1.12 (s, 3H), 1.02 (br d, J=13.6 Hz, 1H), 0.69 (s, 3H).

LCMS: Rt=0.590 min in 1.5 min chromatography, 5-95AB, purity 95.249%, LCMS ESI+ calcd, for C₃₅H₄₆N₂O₅ [M]+ 574.3, found [M+H]+ 575.3.

HPLC: Rt=2.906 min in 4 min chromatography, 10-80AB, purity 98.90%.

### Example 5: Synthesis of N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-N-methyl-2-(4-methylpiperazin-1-yl)-2-oxoacetamide (Compound 16)

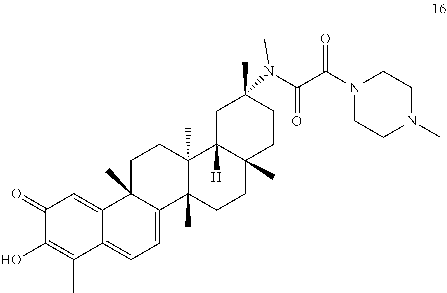

Compound 16 was prepared via the synthesis summarized in Scheme 5 (below).

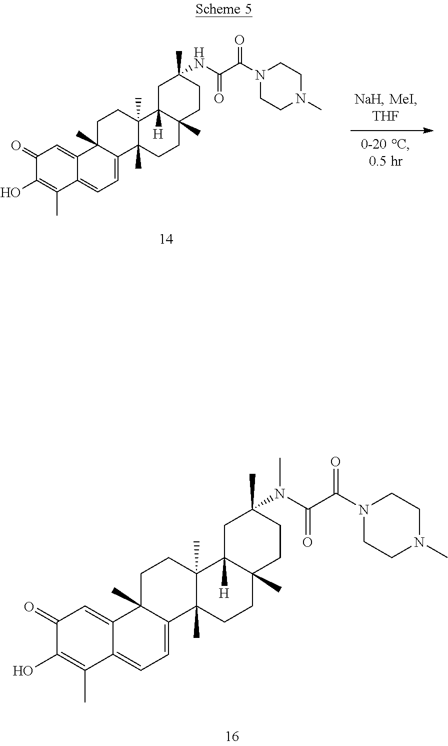

### Step 1

To a solution N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-2-(4-methylpiperazin-1-yl)-2-oxoacetamide (compound 14) (180 mg, 313 μmol) in THF (5 mL) was added Mel (44.0 mg, 312 μmol). Then NaH (75.0 mg, 1.88 mmol, 60% in mineral oil) was added at 0° C. under N2. The resulting mixture was stirred at 20° C. for 0.5 hour. The reaction mixture was poured into sat. aq. NH₄Cl solution (20 mL) slowly at 0° C., then extracted with DCM (3×20 mL). The organic phase was dried over anhydrous Na₂SO₄, filtered, and concentrated under reduced pressure. The crude product was purified by column chromatography (dichloromethane:methanol=1:0 to 50/1; Rf=0.54) to give N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-N-methyl-2-(4-methylpiperazin-1-yl)-2-oxoacetamide (compound 16) (5.75 mg, 2.86% yield) as yellow solid.

¹H NMR: CDCl₃ 400 MHz δ=7.12-6.87 (m, 2H), 6.53 (d, J=1.0 Hz, 1H), 6.36 (d, J=7.1 Hz, 1H), 5.81-5.79 (m, 1H), 3.73 (q, J=7.0 Hz, 4H), 3.41 (br dd, J=3.8, 12.9 Hz, 4H), 2.87 (s, 3H), 2.50-2.37 (m, 4H), 2.31 (s, 3H), 2.21 (s, 3H), 2.17-1.97 (m, 3H), 1.96-1.81 (m, 4H), 1.46 (s, 3H), 1.37-1.31 (m, 4H), 1.23 (s, 3H), 1.20-1.09 (m, 5H), 0.87 (s, 3H).

LCMS: Rₜ=0.492 min in 1.5 min chromatography, 5-95AB, purity 96.29%, LCMS ESI+ calcd, for C₃₆H₅₁N₃O₄ [M]+ 589.4, found [M+H]+ 590.4.

HPLC: Rₜ=2.187 min in 4 min chromatography, 10-80AB, purity 91.7%.

### Example 6: Synthesis of N1-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-N2-(1-methylazetidin-3-yl)oxalamide (Compound 17)

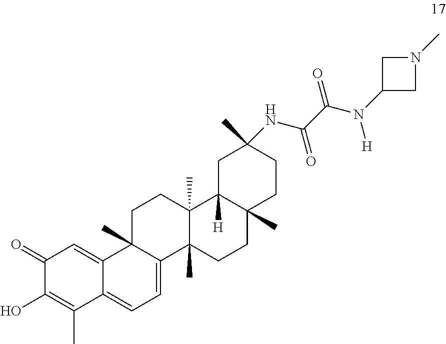

Compound 17 was prepared via the synthesis summarized in Scheme 6 (below).

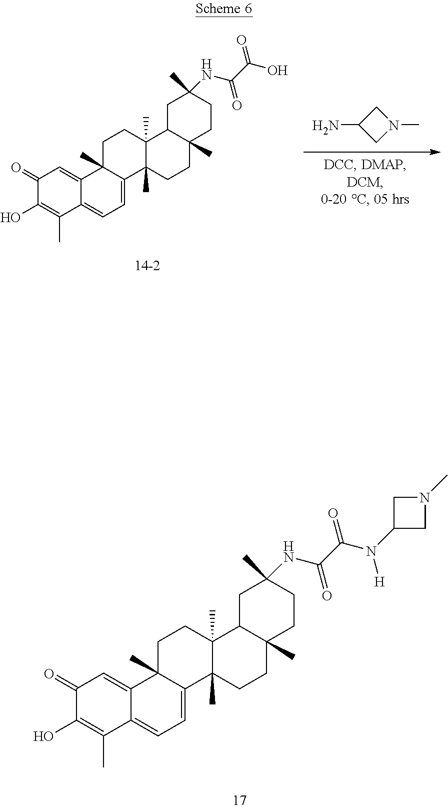

### Step 1

To a solution of 2-(((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)amino)-2-oxoacetic acid (compound 14-2) (200 mg, 405 μmol) in DCM (5 mL) was added DCC (125 mg, 608 μmol) at 0° C. Then 1-methylazetidin-3-amine (38.4 mg, 446 μmol) and DMAP (24.8 mg, 203 μmol) was added. The resulting mixture was stirred at 20° C. for 0.5 hour. The reaction mixture was poured into water (5 mL). The aqueous phase was extracted with DCM (3×3 mL). The combined organic phase was dried over anhydrous Na₂SO₄, filtered, and concentrated under reduced pressure. The residue was purified by column chromatography (dichloromethane:methanol=1/0 to 50/1, Rf=0.40) to give a crude product (100 mg). The crude product was diluted with EtOAc (10 mL) and washed with aq. HCl solution (0.02 M, 10 mL×2). The organic phase was dried over anhydrous Na₂SO₄, filtered and concentrated under reduced pressure to give N1-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahydropicen-2-yl)-N2-(1-methylazetidin-3-yl)oxalamide (compound 17) (60.0 mg, 22.2% yield) as a yellow solid.

¹H NMR: CDCl₃ 400 MHz δ=7.74 (br d, J=8.5 Hz, 1H), 7.23 (s, 1H), 7.01 (dd, J=0.9, 7.1 Hz, 1H), 6.52 (d, J=1.0 Hz, 1H), 6.35 (d, J=7.1 Hz, 1H), 4.39 (dd, J=6.6, 14.9 Hz, 1H), 3.63 (t, J=7.5 Hz, 2H), 2.95 (q, J=6.7 Hz, 2H), 2.63 (br d, J=15.4 Hz, 1H), 2.32 (s, 3H), 2.22-2.12 (m, 4H), 1.90-1.69 (m, 10H), 1.44 (d, J=11.8 Hz, 5H), 1.29-1.25 (m, 6H), 1.13 (s, 3H), 1.04 (br d, J=14.0 Hz, 1H), 0.91-0.86 (m, 1H), 0.68 (s, 3H).

LCMS: Rt=0.437 min in 1.5 min chromatography, 5-95AB, purity 92.577%, LCMS ESI+ calcd, for C₃₄H₄₇N₃O₄ [M]+ 561.4. found [M+H]+ 562.5.

HPLC: Rt=2.206 min in 4 min chromatography, 10-80AB, purity 91.886%.

### Example 7: Synthesis of N′-[(2R,4aS,6aR,6aS,14aS,14bR)-10-methoxy-2,4a,6a,6a,9,14a-hexamethyl-11-oxo-1,3,4,5,6,13,14,14b-octahydropicen-2-yl]-N,N,N′-trimethyl-oxamide (Compound 22)

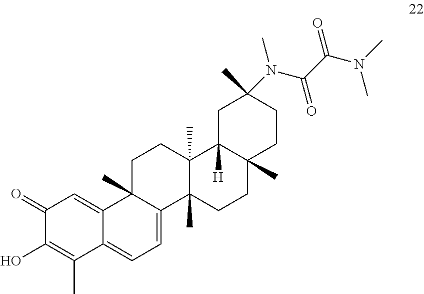

Compound 22 was prepared via the 2-step synthesis summarized in Scheme 7 (below).

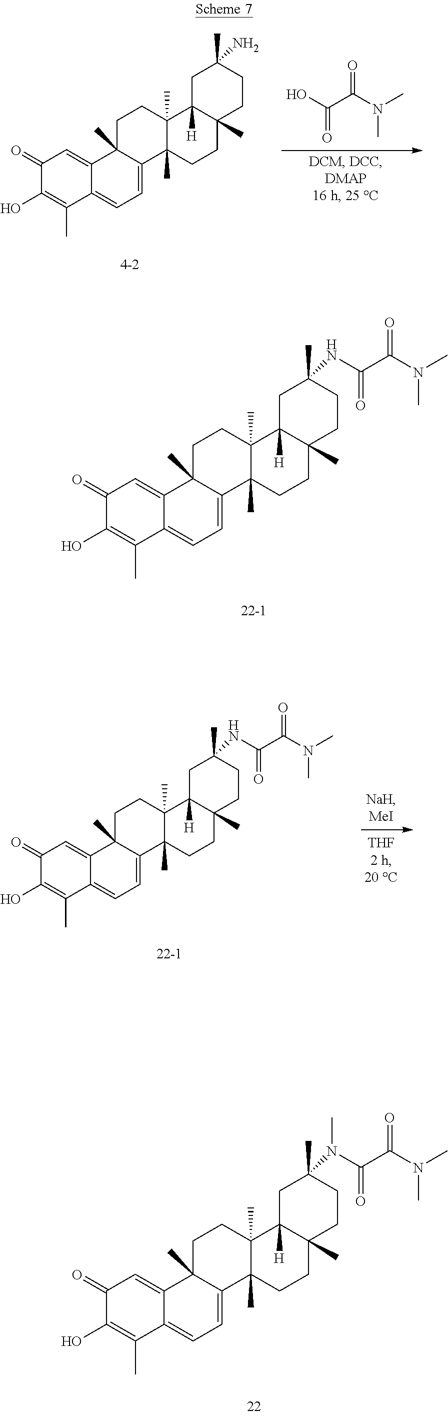

### Step 1

### Synthesis of N-[(2R,4aS,6aR,6aS,14aS,14bR)-10-hydroxy-2,4a,6a,6a,9,14a-hexamethyl-11-oxo-1,3,4,5,6,13,14,14b-octahydropicen-2-yl]-N′,N′-dimethyl-oxamide (Compound 22-1)

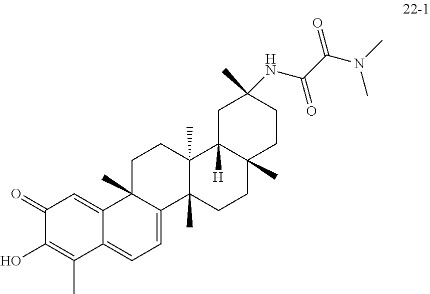

To a solution of (6aS,6bS,8aS,11R,12aR,14aR)-11-amino-3-hydroxy-4,6a,6b,8a,11,14a-hexamethyl-7,8,9,10,12,12a,13,14-octahydropicen-2-one (50 mg, 119 μmol) and 2-(dimethylamino)-2-oxo-acetic acid (18 mg, 154 μmol) in DCM (1 mL) was added DCC (36.7 mg, 178 μmol) and DMAP (7.24 mg, 59.3 μmol) at 25° C. The reaction was stirred at 25° C. for 16 hours. The residue was poured into ice-water (30 mL) and the aqueous phase was extracted with DCM (3×10 mL). The combined organic phase was washed with brine (30 mL), dried over anhydrous Na₂SO₄, filtered, and concentrated in vacuum. The residue was purified by silica gel chromatography eluted with Petroleum ether/Ethyl acetate=1:1 to give a crude (90 mg). The crude was further purified by prep-HPLC (column: Phenomenex Gemini-NX C1875*30 mm*3 μm; mobile phase: [water (0.225% FA)-ACN]; B %: 60%-90%, 7 min) to give N-[(2R,4aS,6aR,6aS,14aS,14bR)-10-hydroxy-2,4a,6a,6a,9,14a-hexamethyl-11-oxo-1,3,4,5,6,13,14,14b-octahydropicen-2-yl]-N′,N′-dimethyl-oxamide (compound 22-1) (2.0 mg, purity: 100%) as a yellow solid.

¹H NMR: CDCl₃ 400 MHz δ=7.13 (s, 1H), 7.04-6.93 (m, 2H), 6.51 (s, 1H), 6.35 (d, J=7.6 Hz, 1H), 3.37 (s, 3H), 2.93 (s, 3H), 2.77 (d, J=14.0 Hz, 1H), 2.21 (s, 3H), 2.01-1.65 (m, 10H), 1.47-1.40 (m, 7H), 1.30-0.99 (m, 9H), 0.73 (s, 3H).

LCMS: Rₜ=1.120 min in 1.5 min chromatography, 5-95AB, purity 100%, LCMS ESI+ calcd, for C₃₂H₄₄N₂O₄ [M]⁺ 520.3, found [M+H]⁺ 521.1.

HPLC: Rₜ=6.12 min in 8 min chromatography, 10-80AB, purity 100%.

### Step 2

### Synthesis of N′-[(2R,4aS,6aR,6aS,14aS,14bR)-10-methoxy-2,4a,6a,6a,9,14a-hexamethyl-11-oxo-1,3,4,5,6,13,14,14b-octahydropicen-2-yl]-N, N,N′-trimethyl-oxamide (Compound 22)

To a solution of N-[(2R,4aS,6aR,6aS,14aS,14bR)-10-hydroxy-2,4a,6a,6a,9,14a-hexamethyl-11-oxo-1,3,4,5,6,13,14,14b-octahydropicen-2-yl]-N′,N′-dimethyl-oxamide (300 mg, 576 μmol) in THF (3 mL) was added NaH (69.13 mg, 1.73 mmol, 60% in mineral oil) at 20° C. and stirred for 30 mins. Mel (81.8 mg, 576 μmol) was added at 20° C. and the reaction mixture was stirred for 2 hours. The reaction mixture was poured into H₂O (50 mL). The mixture was extracted with DCM (4×10 mL). The combined organic phase was washed with brine (30 mL), dried over anhydrous Na₂SO₄, filtered, and concentrated in vacuum. The crude product was purified by silica gel chromatography (eluted with ethyl acetate/petroleum ether=0 to 100%) to give N′-[(2R,4aS,6aR,6aS,14aS,14bR)-10-hydroxy-2,4a,6a,6a,9,14a-hexamethyl-11-oxo-1,3,4,5,6,13,14,14b-octahydropicen-2-yl]-N,N,N′-trimethyl-oxamide (compound 22) (20 mg, 6% yield) as yellow solid.

¹H NMR: CDCl₃ 400 MHz δ=7.02-6.95 (m, 2H), 6.51 (d, J=1.2 Hz, 1H), 6.34 (d, J=7.2 Hz, 1H), 3.48-3.33 (m, 1H), 2.93-2.89 (m, 6H), 2.84 (s, 3H), 2.20 (s, 3H), 2.15-1.65 (m, 11H), 1.59-1.52 (m, 2H), 1.46-1.41 (m, 6H), 1.30 (s, 3H), 1.16 (s, 3H), 1.09-1.02 (m, 1H), 0.86 (s, 3H).

LCMS: Rₜ=1.119 min in 1.5 min chromatography, 5-95AB, purity 96.0%, LCMS ESI+ calcd, for C₃₃H₄₆N₂O₄ [M]⁺ 534.3, found [M+H]⁺ 535.4.

HPLC: Rₜ=2.103 min in 4 min chromatography, 5-95AB, purity 92.0%.

### Example 8: Synthesis of Compound 14-4

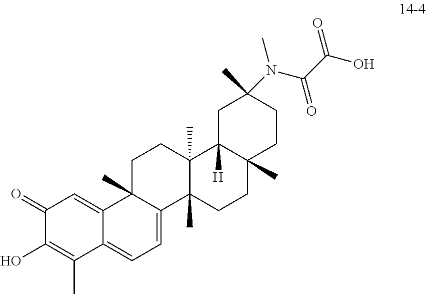

Compound 14-4 can be prepared via a 6-step synthesis as summarized in Scheme 8 (below).

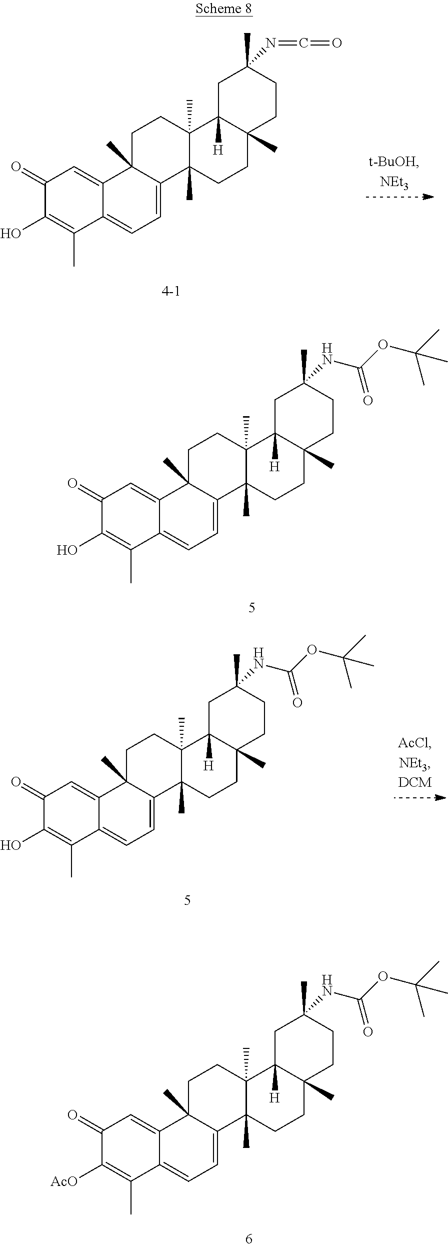

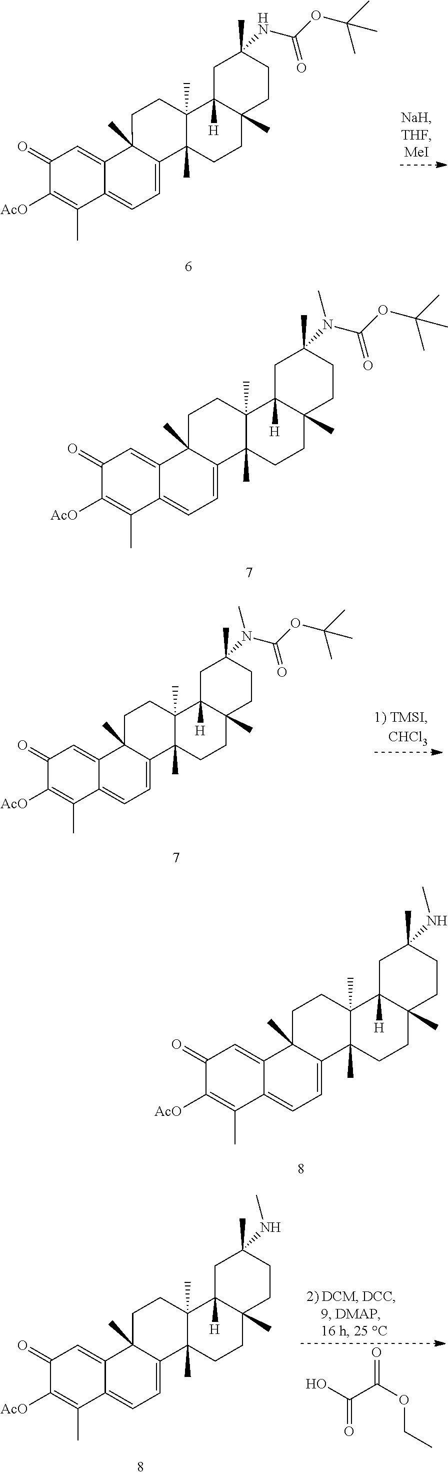

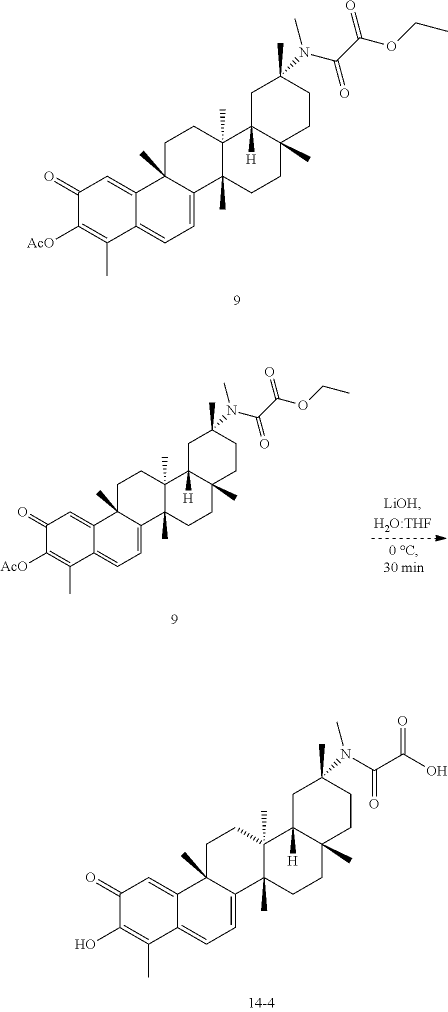

### Example 9: Synthesis of Compound 54

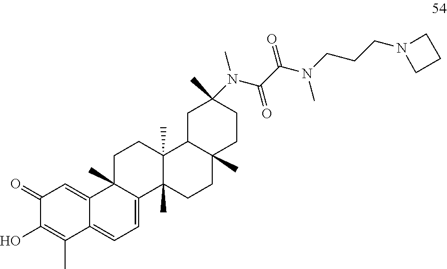 [SMILES: CC1=C(O)C(=O)C=C2C1=CC=C1[C@@]2(C)CC[C@@]2(C)C3C[C@](C)(N(C)C(=O)C(=O)N(C)CCCN4CCC4)CC[C@]3(C)CC[C@]12C]

Compound 54 can be prepared from the compound 14-4 via the synthesis summarized in Scheme 9:

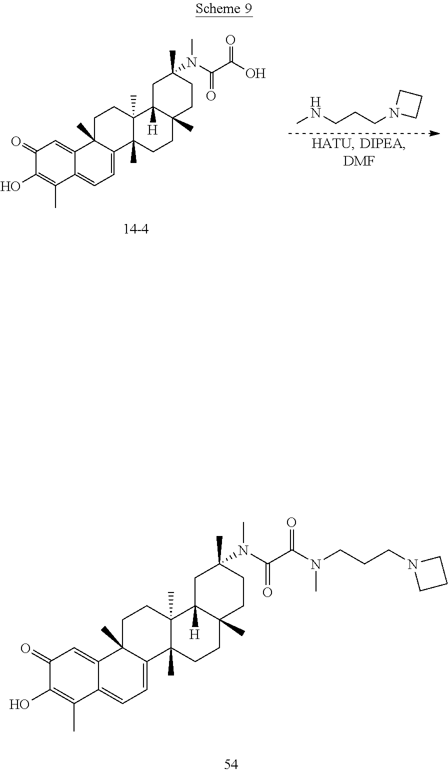

### Example 10: Synthesis of Compound 55

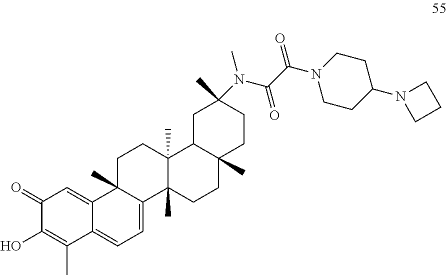 [SMILES: CC1=C(O)C(=O)C=C2C1=CC=C1[C@@]2(C)CC[C@@]2(C)C3C[C@](C)(N(C)C(=O)C(=O)N4CCC(N5CCC5)CC4)CC[C@]3(C)CC[C@]12C]

Compound 55 can be prepared from compound 14-4 via the synthesis summarized in Scheme 10:

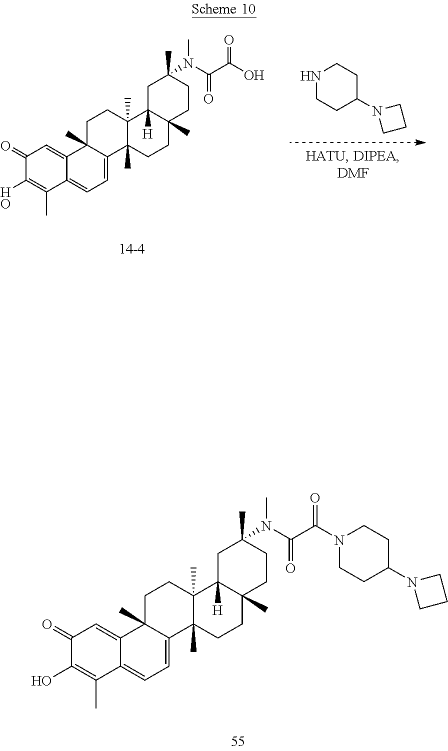

### Example 11: Synthesis of Compound 56

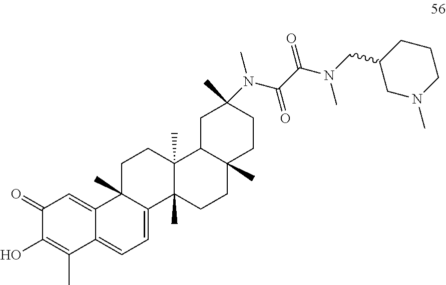 [SMILES: CC1=C(O)C(=O)C=C2C1=CC=C1[C@@]2(C)CC[C@@]2(C)C3C[C@](C)(N(C)C(=O)C(=O)N(C)CC4CCCN(C)C4)CC[C@]3(C)CC[C@]12C]

Compound 56 can be prepared from compound 14-4 via the synthesis summarized in Scheme 11:

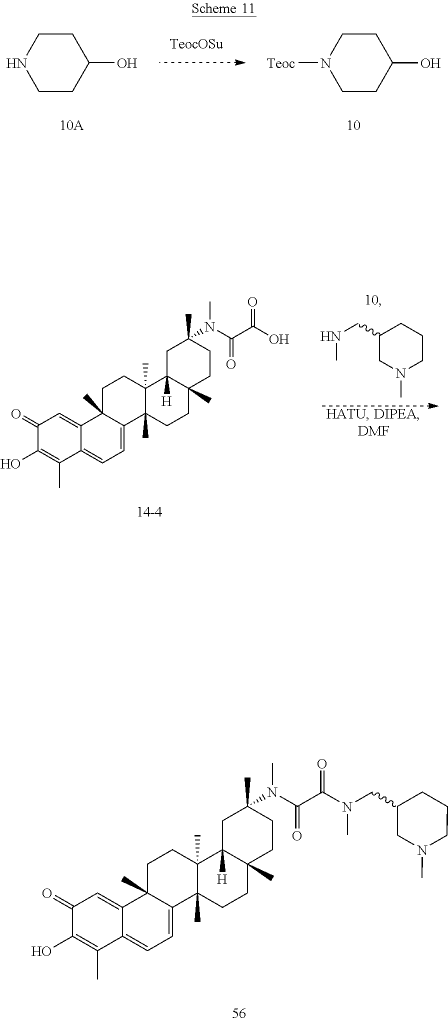

### Example 12: Synthesis of Compound 57

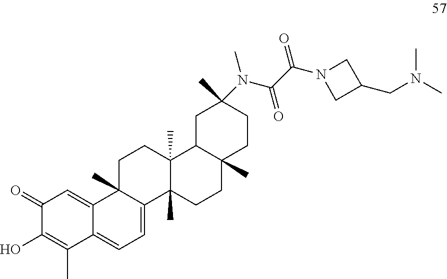 [SMILES: CC1=C(O)C(=O)C=C2C1=CC=C1[C@@]2(C)CC[C@@]2(C)C3C[C@](C)(N(C)C(=O)C(=O)N4CC(CN(C)C)C4)CC[C@]3(C)CC[C@]12C]

Compound 57 can be prepared from compound 14-4 via the synthesis summarized in Scheme 12:

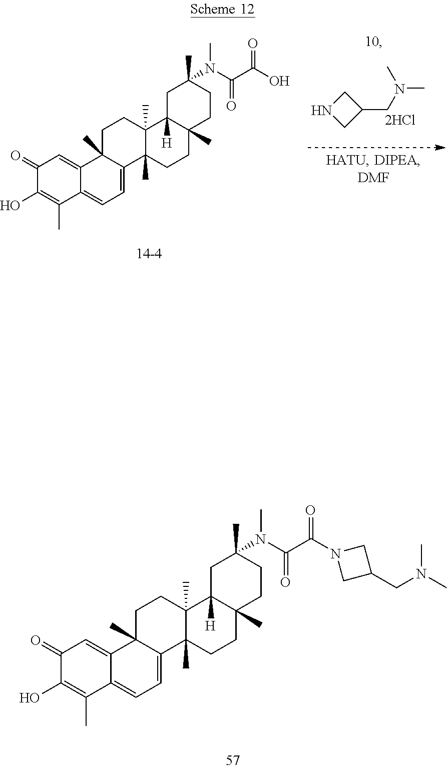

### Example 13: Inhibition of NF-kB Signaling by Celastrol Derivative Compounds

This example illustrates a study of the inhibition of NF-κB signaling by the following exemplary celastrol derivative compounds of the present disclosure: compound 14, compound 15, compound 16, compound 17, and compound 22.

### Materials and Methods

HepG2 NF-kB-Luc reporter cells stimulated with IL-1β (20 ng/ml) and the relative NF-kB luciferase activity (RLU) was determined for each of celastrol (compound 1) and each of the exemplary derivative compounds at the following eight point concentrations: 10 μM, 5 μM, 2.5 μM, 1.25 μM, 0.63 μM, 0.31 μM, 0.16 μM, 0.016 μM, and 0 μM). Assay values were plotted (not shown) and IC₅₀ inhibitory concentrations were calculated after normalization using Prizm 9.0 software.

### Results

Data from the NF-kB inhibition assays was analyzed and the resultant IC₅₀ values for the celastrol derivative compounds and their % IC₅₀ values relative to celastrol (compound 1; IC₅₀=667 nM) are summarized in Table 4 (below).

TABLE 4

| Celastrol | | % IC₅₀ relative |
|---|---|---|
| Derivative | NF-kB IC₅₀ | to |
| Compound | (nM) | Celastrol |
| 14 | 1157 | 115 |
| 15 | 942 | 93 |
| 16 | 1150 | 114 |
| 17 | 1643 | 163 |
| 22 | 268 | 51 |

### Example 14: Anti-Inflammatory Potential of a Celastrol Derivative Compound

This example illustrates a study of the anti-inflammatory potential of an exemplary celastrol derivative of the present disclosure (compound 22) using an NF-κB luciferase promoter reporter gene assay in the human liver cancer cell line, HepG2.

### Materials and Methods

NF-kB luciferase reporter assay: The celastrol derivative, compound 22 (prepared as described in Example 13) and the parent molecule, compound 1 (celastrol), were freshly dissolved in DMSO. HepG2 NF-κB reporter cells were seeded (2.5×10⁴/well) in 96-well plate and then pre-treated with either compound 22 or compound 1 at the 8-point concentrations of 10, 5, 2.5, 1.25, 0.63, 0.31, 0.16, 0.016, or 0 μM, respectively, and the NF-κB promoter was activated by adding IL-1β (20 ng/ml) to the cells. After 18 hours, the cells were harvested by adding the 1× passive cell lysis buffer (Promega) and followed by measurement of the relative NF-kB luciferase activity (RLU) using GloMax discover microplate reader (Promega). Inhibitory concentrations (IC₅₀) after normalization using Prizm 9.0 software.

LPS-induced NF-kB target protein expression: Bone marrow-derived dendritic cells (BMDCs) were pre-treated with compound 22 or compound 1 at three concentrations (10 nM, 25 nM, 50 nM) for an hour and then treated overnight with LPS (500 ng/ml). The next day, the cells were subjected to western blotting for iNOS, COX2, NLRP3, and IL-1β.

### Results

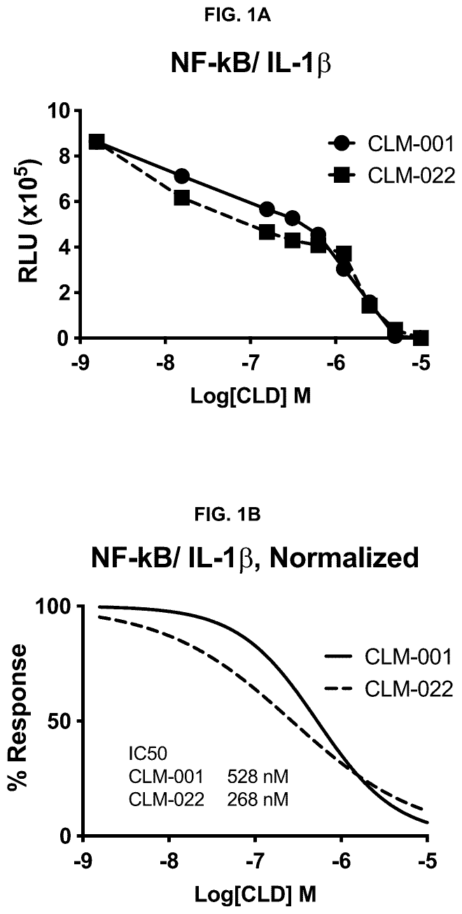

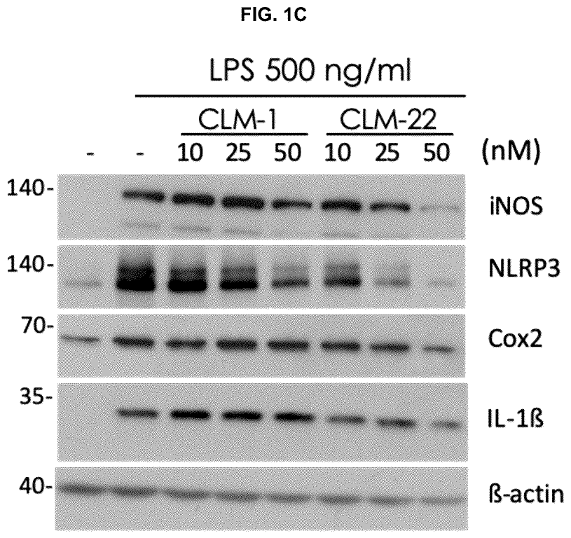

As shown by the plot of results depicted in FIG. 1A, the celastrol derivative compound 22 possesses a robust inhibitory potential for IL-1β-induced NF-kB inflammatory signaling pathway superior to celastrol (compound 1) at low concentrations. As shown by FIG. 1B, analysis by the normalized percentage response curve indicates that compound 22 has an IC₅₀ of 268 nM for NF-κB promoter activity, a value that is 50% lower than that of compound 1 (IC₅₀=528 nM). Further, as shown by the western blot images shown in FIG. 1C, the celastrol derivative compound, compound 22, markedly inhibited expression of the LPS-induced NF-kB target proteins iNOS, COX2, NLRP3, and IL-1β in a dose-dependent fashion.

### Example 15: Anti-Inflammatory Potential of a Celastrol Derivative Compound

This example illustrates a study of the anti-inflammatory potential of an exemplary celastrol derivative of the present disclosure (compound 22) using specific NLRP3 inflammasome assays that employ bone marrow-derived dendritic cells (BMDCs) activated with LPS+ATP or LPS+Nigericin.

### Materials and Methods

### NLRP3 Inflammasome Assays

1. BMDCs were treated with LPS (100 ng/ml) for 3 hours (Signal 1) to prime NLRP3 inflammasome, and then treated with celastrol (compound 1) or the celastrol derivative compound, compound 22, at concentrations of 50 nM, 100 nM, and 500 nM. After 30 minutes Nigericin (10 μM) or ATP (5 mM) was added (Signal 2) and the level of NLRP3-dependent inflammasome activity was determined by western blotting the treated cells with ASC complex (>48 kDa). Also, the level of secreted IL-1β in the conditioned media represents an active caspase-1, a downstream molecule of NLRP3 inflammasome, which cleaves pro-IL1β resulting in the secreted form of mature IL-1β.

2. The secreted form of IL-1δ, a NLRP3 inflammasome dependent cytokine, was measured by ELISA assay using the conditioned media of BMDC treated with compound 1 (CLM-1) or compound 22 (CLM-022) at concentrations of 63, 125, 250, 500 nM under NLRP3 inflammasome activation induced either by LPS+ATP or LPS+Nigericin.

3. The secreted form of TNF-α, a NLRP3 inflammasome independent cytokine, induced by LPS+ATP or LPS+Nigericin were measured by ELISA assay using the conditioned media of BMDC treated with compound 1 (CLM-1) or compound 22 (CLM-022) at concentrations of 63, 125, 250, 500 nM under NLRP3 inflammasome activation induced either by LPS+ATP or LPS+Nigericin.

### Results

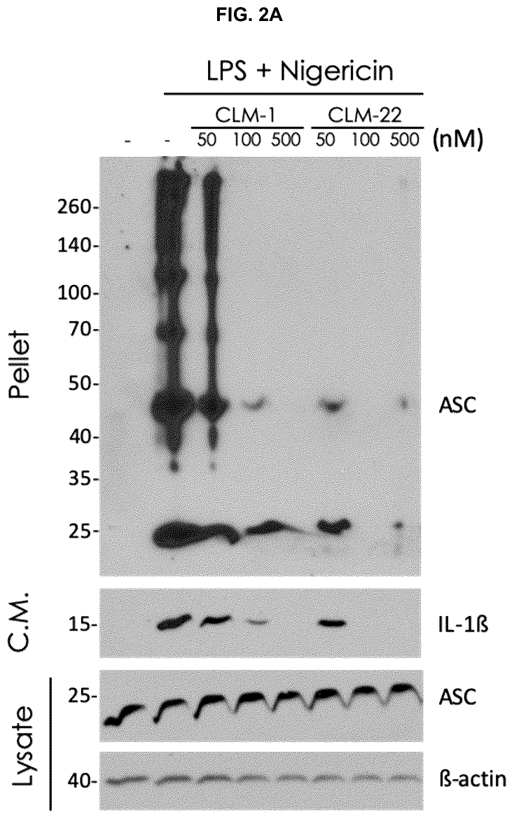

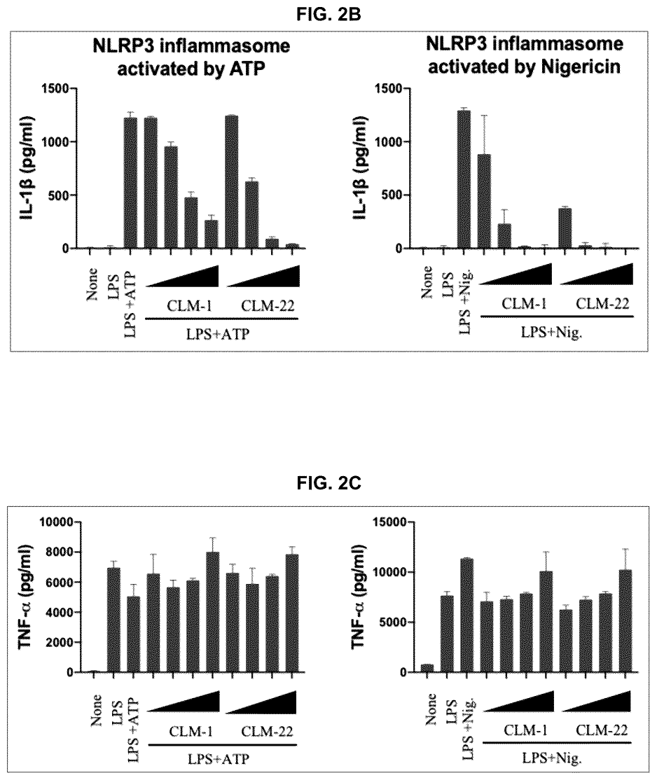

Compound 22 exhibited a robust inhibitory function on NLRP3 inflammasome activity that was better than that observed for the parental molecule celastrol (compound 1). As shown by the results depicted in FIG. 2A, compound 22 suppressed ASC complex formation more strongly than compound 1. Additionally, as shown by the plots depicted in FIG. 2B, compound 22 more strongly inhibited IL-1β secretion induced by both LPS+ATP and LPS+Nigericin. As shown in FIG. 2C, compound 22 (and compound 1) exhibited no significant suppressive effect on TNF-α inflammatory cytokine secretion under identical NLRP3 inflammasome activation. The lack of a suppressive effect on TNF-α secretion, which is independent of NLRP3 inflammasome activity, indicates specificity of compound 22 toward NLRP3 inflammasome. Therefore, compound 22 has capacity of inhibiting NF-κB inflammatory responses and also specifically suppresses IL-1β secretion induced by NLRP3 inflammasome in BMDC.

### Example 16: Inhibition of NLRP3 Inflammasome in Human THP-1 Cells by a Celastrol Derivative Compound

This example illustrates a study of the anti-inflammatory potential of an exemplary celastrol derivative of the present disclosure (compound 22) using an NLRP3 inflammasome assay in THP-1, a human monocytic cell line, activated with LPS+Nigericin in the presence of compound 22 or the known NLRP3 inhibitor compound, MCC950 (CAS 256373-96-3), as a benchmark molecule.

### Materials and Methods

### ASC Complex Formation in THP-1 Cells

Human THP-1 cells were treated with LPS+Nigericin as shown previously (FIG. 2) where increasing dose of compound 1, compound 22, or MCC950 were added as indicated. To demonstrate the relative NLRP3 inflammasome activity, the level of ASC complex formation was compared by western blotting.

### Detection of ASC Complex and Caspase-1 Activity in THP-1 Cells by Amnis

Amnis Imaging flow cytometry (Luminex Co.) is a high-throughput, single-cell, fluorescence-based image analysis method which can detect inflammasome by acquiring images of cellular distribution of ASC complex and active caspase-1. The THP-1 cells were treated with LPS+Nigericin in the presence of inhibitors. The treated cells were subjected to ASC staining and visualization of active caspase-1 using FLICA, a caspase-1 substrate exerting fluorescence upon cleavage. Finally, the ASC+/FLICA+ cells were counted as THP-1 cells with active NLRP3 inflammasome complex.

### Results

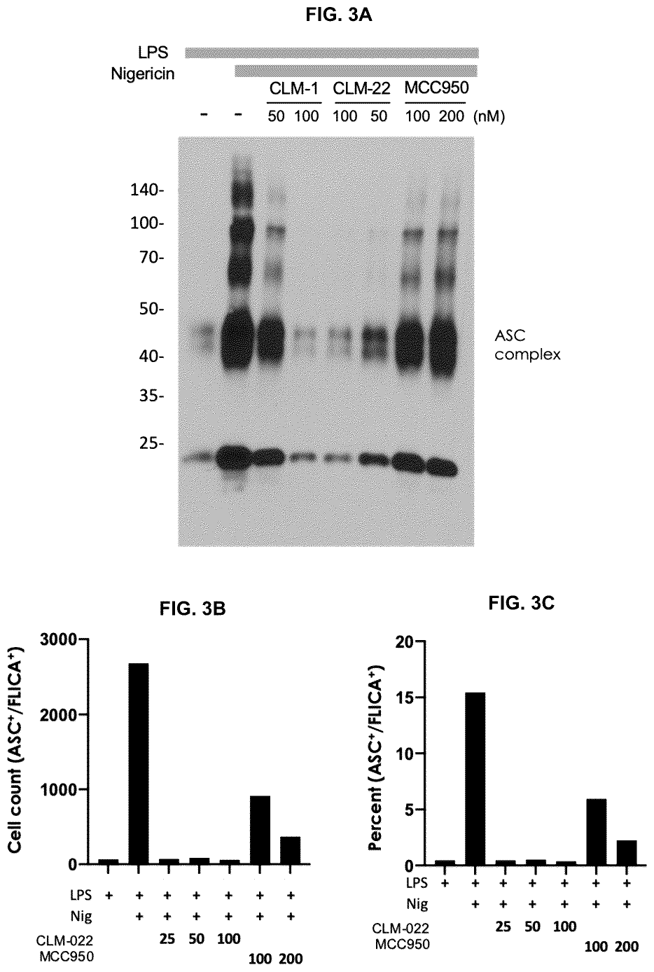

THP-1 human monocytic cells form a strong ASC complex in response to LPS+Nigericin treatment. As shown by the western blots of FIG. 3A, the presence of compound 22 at 50 nM and 100 nM concentration strongly inhibited this ASC complex formation with an inhibitory capacity that was stronger than that observed for celastrol (compound 1) and MCC950, the benchmark molecule. As shown by the Amnis Imaging results of FIG. 3B, the strong inhibitory effect observed for compound 22 was verified further by counting of THP-1 cells carrying cellular ASC+FLICA+ specks. As shown by the plots of FIG. 3C, compound 22 was observed to exert a strong inhibitory function at concentrations as low as 25 nM, while the benchmark molecule, MCC950 displayed 5~10-fold lesser inhibitory potency at 100 nM concentration.

### Example 17: Inhibition of Inflammation by a Celastrol Derivative Compound in an LPS-Induced Septic Shock Mouse Model

This example illustrates a study of the inhibitory function of an exemplary celastrol derivative of the present disclosure (compound 22) against LPS-induced acute inflammation in vivo, we set up LPS-induced septic shock mouse model (Yu et al., 2017) and detected the expression levels of inflammatory cytokines in colon.

### Materials and Methods

Mice (n=3 per group) were administered intraperitoneally (i.p.) with control vehicle, compound 1 (1 mg/kg), or compound 22 (1 mg/kg). After 1 hour, mice were administered by i.p. injection a sublethal dose of LPS (25 mg/kg). Four hours later, the mice were subjected to blood collection and harvesting colonic mucosa by scraping colon. Cytokine expression was measured by conducting ELISA (Cat #900-T54, PeproTech) for TNF-α on blood serum samples and qPCR for the cytokines, IL-1 (TaqMan, Mm00434228), IL-6 (TaqMan, Mm00446190), TNF-α (TaqMan, 00443258), iNOS (TaqMan, 00440502), on colonic mucosa samples.

### Results

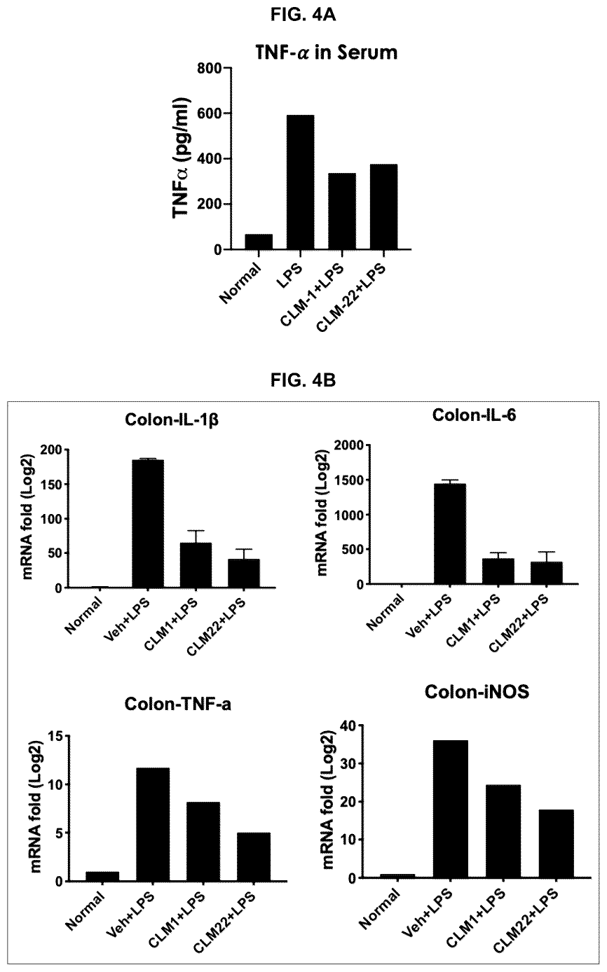

As shown by the results depicted in the plots of FIGS. 4A and 4B, the celastrol derivative compound, compound 22 successfully inhibited LPS-induced acute inflammation responses in the mouse septic-shock model which efficiently suppressed expression of an inflammatory mediator and the inflammatory cytokines including IL-1, IL-6, TNF-α, and INOS in the blood serum and colon mucosa to levels comparable to or lower than the levels observed in the group administered celastrol (Compound 1).

### Example 18: Comparative NLRP3 Inflammasome Inhibitory Activity of CLM-022 and MCC950 in Mouse and Human Macrophages

This example illustrates a comparative study of CLM-022 and MCC950 in inhibiting NLRP3 inflammasome activity in mouse and human macrophages.

### Materials and Methods

### A. BMDM Cell Study

Bone marrow-derived macrophages (BMDMs) were treated with LPS (100 ng/ml) for 3 hours to induce the expression of inflammatory genes, including TNF-α, NLRP3, and pro-IL-1β. Subsequently, the BMDMs were treated with CLM-022 or MCC950 in a dose range of 0-10 μM for 30 minutes. Then, to fully activate NLRP3-dependent inflammasome activity and cytokine secretion, Nigericin (10 μM) was added to the BMDMs for an additional 30 minutes.

### B. THP-1 Cell Study

To confirm the inhibitory effect of CLM-022 in human macrophages, THP-1 cells, a human leukemia monocytic cell line, were differentiated into macrophages using phorbol 12-myristate-13-acetate (PMA, 100 ng/ml) treatment for one day. The THP-1 macrophages were then treated with LPS (100 ng/ml) for 3 hours to induce the expression of inflammatory genes. Subsequently, the THP-1 cells were treated with CLM-022 or MCC950 in a dose range of 0-10 μM for 30 minutes. Then, to fully activate NLRP3-dependent inflammasome activity, Nigericin (10 μM) was added to the THP-1 cells for an additional 60 min.

### Results

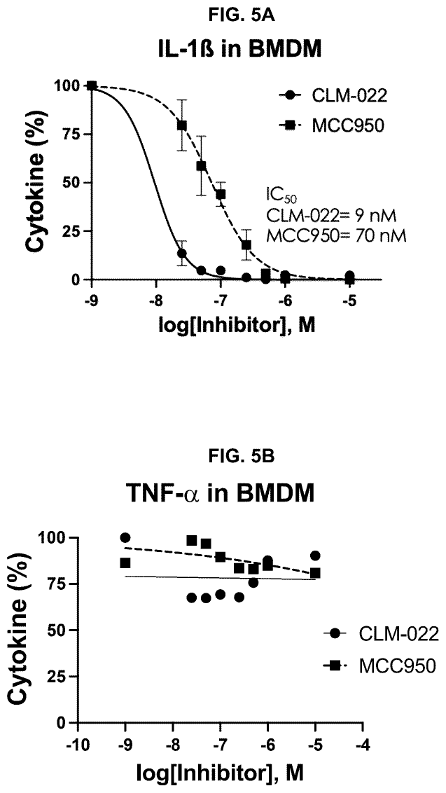

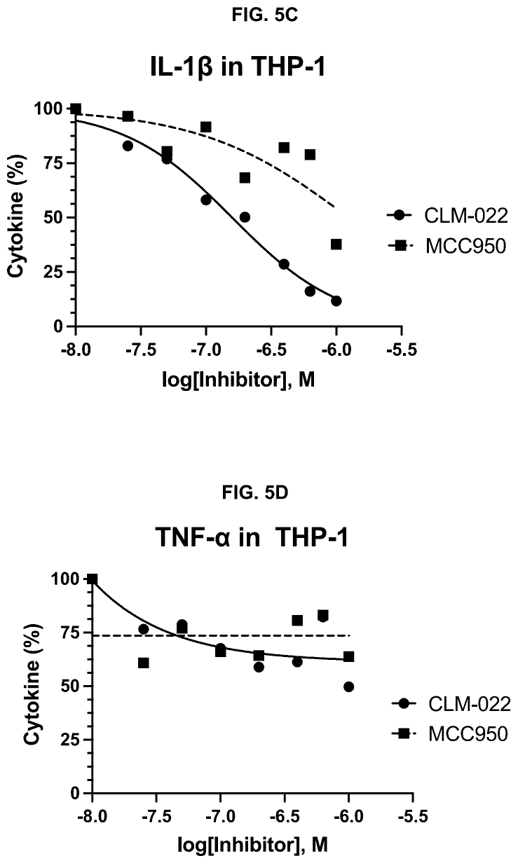

As shown by the results depicted in FIG. 5A, in a dose-dependent manner, CLM-022-treated BMDMs exhibited significant inhibition of IL-1β secretion. The normalized IC₅₀ calculation demonstrated that CLM-022 displayed greater potential in inhibiting IL-1β compared to MCC950 (IC₅₀: CLM-022=9 nM vs. MCC950=70 nM). However, as shown in FIG. 5B, neither CLM-022 nor MCC950 suppressed TNF-α secretion, as TNF-α is not regulated by NLRP3 inflammasome. As shown by the results depicted in FIG. 5C and FIG. 5D, similar to the results obtained with BMDMs, in human THP-1 macrophages CLM-022 robustly inhibited IL-1β secretion but not TNF-α. CLM-022 exhibited a more potent inhibitory potential than MCC950: (IC₅₀: CLM-022=158.9 nM vs. MCC950=1248 nM). These results clearly indicate that CLM-022 possesses superior inflammasome inhibitor potential compared to MCC950, the most advanced NLRP3 inflammasome inhibitor.

### Example 19: DARTS Assay of CLM-022 Protection of NLRP3

The NLRP3 inflammasome is composed of a multi-protein complex and the exact molecular mode of NLRP3 activation remains unclear. Currently, there are no NLRP3 inflammasome inhibitors in the clinic, and it is important to identify the mechanism of action and molecular target of potential small molecules that interact with NLRP3. This example illustrates a study of the ability of CLM-022 to protect the NLRP3 molecule as determined using a drug affinity responsive target stability (DARTS) assay. DARTS assay is a robust method to identify small molecule target protein. In case of a drug-target interaction, the small molecule protects target protein from degradation by proteinase (Pronase) whereas free-proteins may be degraded very efficiently in the given condition.

### Materials and Methods

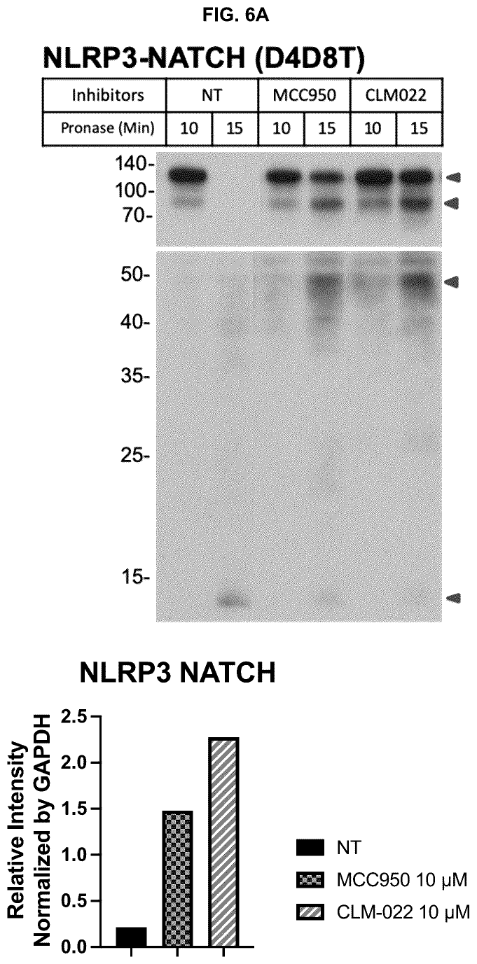

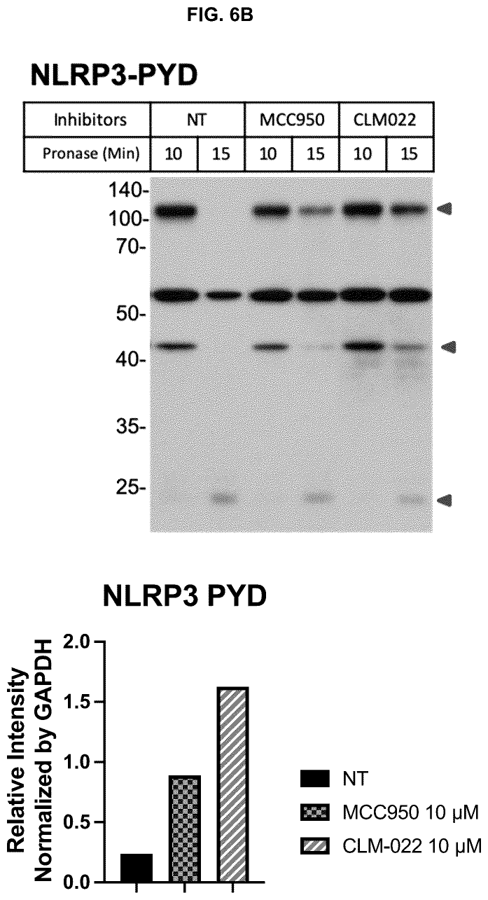

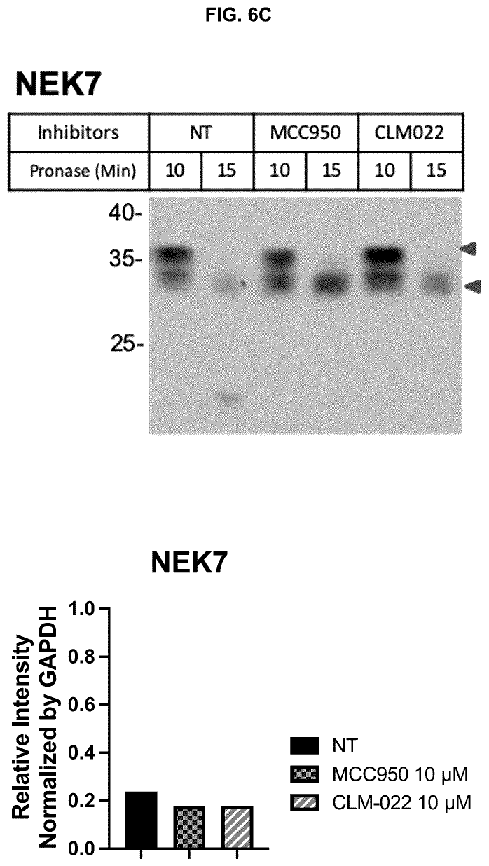

The DARTS assay was performed using THP-1 lysates pretreated with LPS and Nigericin to induce activation of NLRP3 inflammasome. THP-1 cells (1×10⁵/mL) were treated with LPS (100 ng/ml) for 3 hours, followed by 10 μM Nigericin for an additional 30 minutes. Pronase (50-500 ng/μg of protein) was added to the lysate with CLM-022 (10 μM) or MCC950 (10 μM) for the indicated time (10-15 min) at room temperature. The protein components of NLRP3 inflammasome were visualized using antibody recognizing NLRP3-Natch domain (FIG. 6A), NLRP-PYD domain (FIG. 6B), or NEK7 protein (FIG. 6C). The relative band intensities were normalized by GAPDH after densitometry scanning.

### Results

As shown by the results depicted in FIG. 6A, FIG. 6B, and FIG. 6C, CLM-022 markedly protected NLRP3 protein from degradation which was obviously more efficient than that of MCC950. CLM-022, however, did not protect NEK7 from Pronase-induced degradation. This result highlights that CLM-022 would directly interact with NLRP3 protein in a target-specific manner.

### Example 20: Protection of THP-1 Cells from Inflammasome-Induced Pyroptosis

Pyroptosis, a pro-inflammatory form of cell death, is triggered by the activation of NLRP3 inflammasome components (GSDMD and Caspase-1), leading to the release of pro-inflammatory cytokines IL-1β and IL-18, as well as damage-associated molecular patterns (DAMPs). This process elicits a cascade of inflammatory responses, which can persist if dysregulated in affected tissues. Given the significant inhibitory potential of CLM-022 against NLRP3 inflammasome and IL-1β secretion, it was proposed that CLM-022 could effectively enhance cell viability by inhibiting pyroptosis in the context of inflammation and inflammasome activation. This example illustrates a comparative study of the abilities of CLM-022 and MCC950 to protect THP-1 cells from inflammasome-induced pyroptosis.

### Materials and Methods

After LPS and Nigericin treatment, THP-1 cells were treated with either CLM-022 or MCC950 over a dosage range of 0 μM to 10 μM and cell viability was determined by impermeant live/dead staining dye and flow cytometric analysis. Additionally, NLRP3 inflammasome-induced cytotoxicity was investigated by measuring the level of LDH in the conditioned media after the CLM-022 and MCC950 treatments. Further, Western blotting of samples from the THP-1 cell viability assays was carried out to determine levels of the pyroptosis-mediating molecules, GSDMD and Caspase-1.

### Results

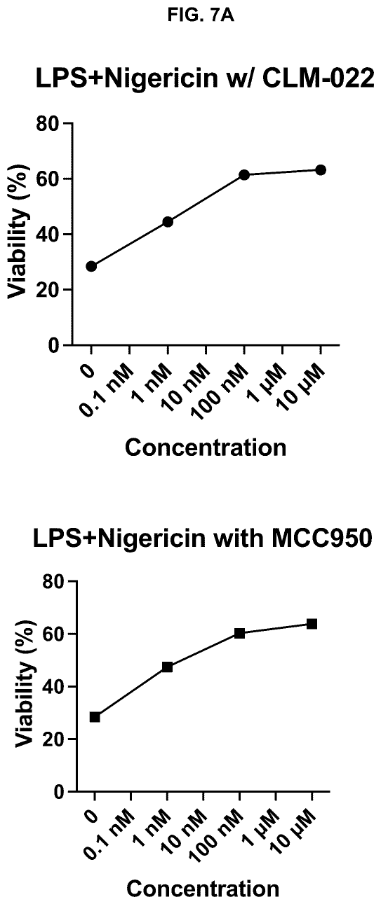

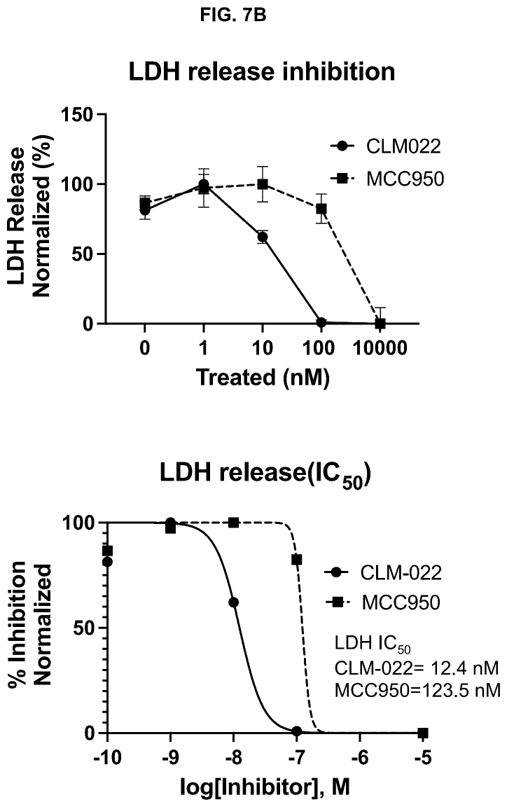

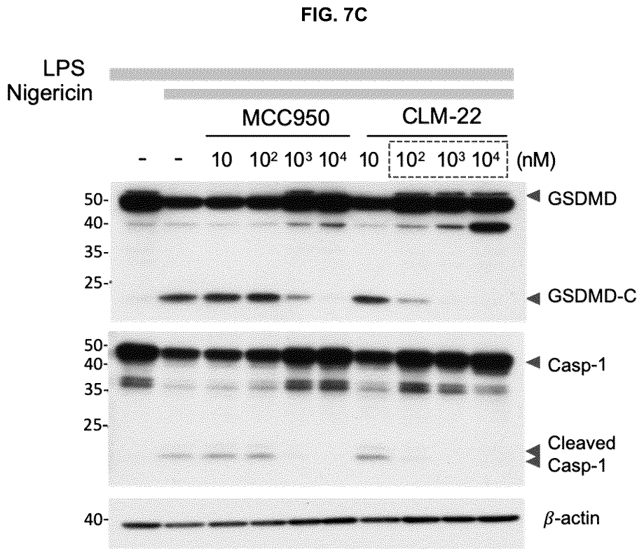

As shown by the results depicted in FIG. 7A, when the THP-1 cells were treated within the dose-range of 0 μM to 10 μM with CLM-022, the cell viability increased by up to 60%. A similar effect was observed with cells treated with MCC950 (FIG. 7A).

As shown by the results depicted in FIG. 7B, consistent with the cell viability assay results, both CLM-022 and MCC950 significantly inhibited release of LDH. Notably, CLM-022 exhibited an inhibitory potential superior to MCC950 even at lower concentrations (FIG. 7B, left). Normalized IC₅₀ values calculated from these LDH release results clearly demonstrated that CLM-022 relative to MCC950 exhibits a 10-fold higher potency (IC₅₀: CLM-022=12.4 nM vs. MCC950=123.5 nM) in preventing pyroptosis.

As shown by the Western blot images depicted in FIG. 7C, CLM-022 inhibited GSDMD and Caspase-1 cleavage at concentrations as low as 100 nM, while MCC950 required 1000 nM for inhibition.

Overall, these comparative analyses unequivocally demonstrated that CLM-022 possesses a greater capacity than the benchmark molecule, MCC950, to inhibit inflammasome-induced pyroptosis in human THP-1 macrophages.

While the foregoing disclosure of the present disclosure has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the disclosure are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

1. A compound of structural formula (I) 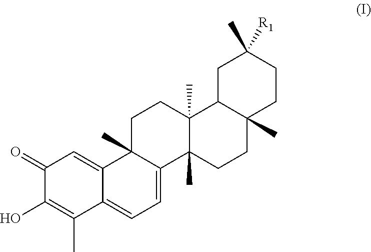
wherein, R₁ is selected from: 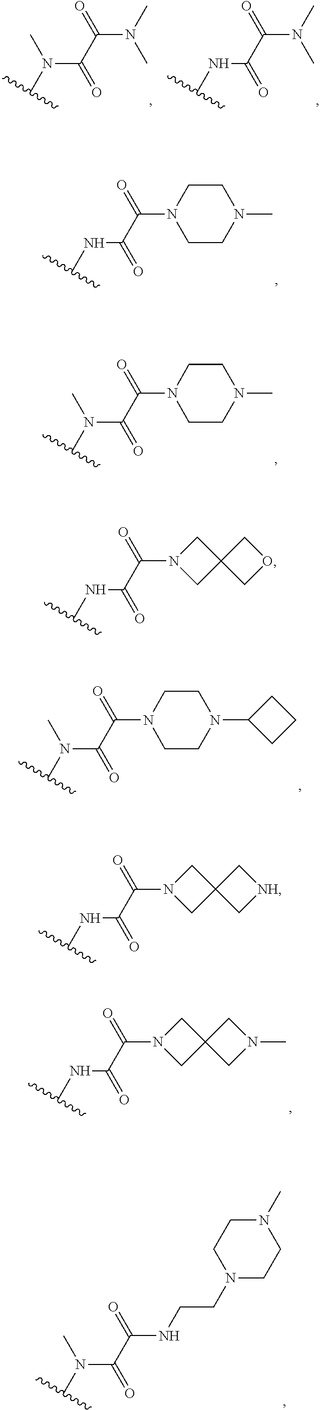 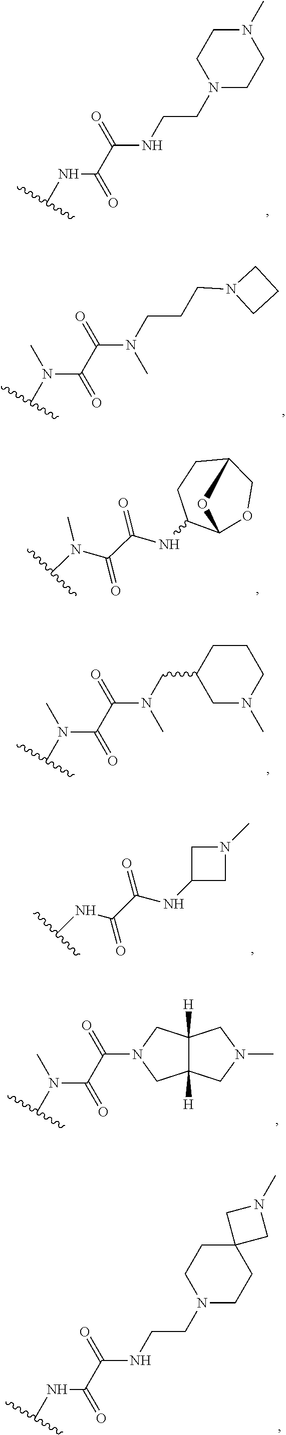 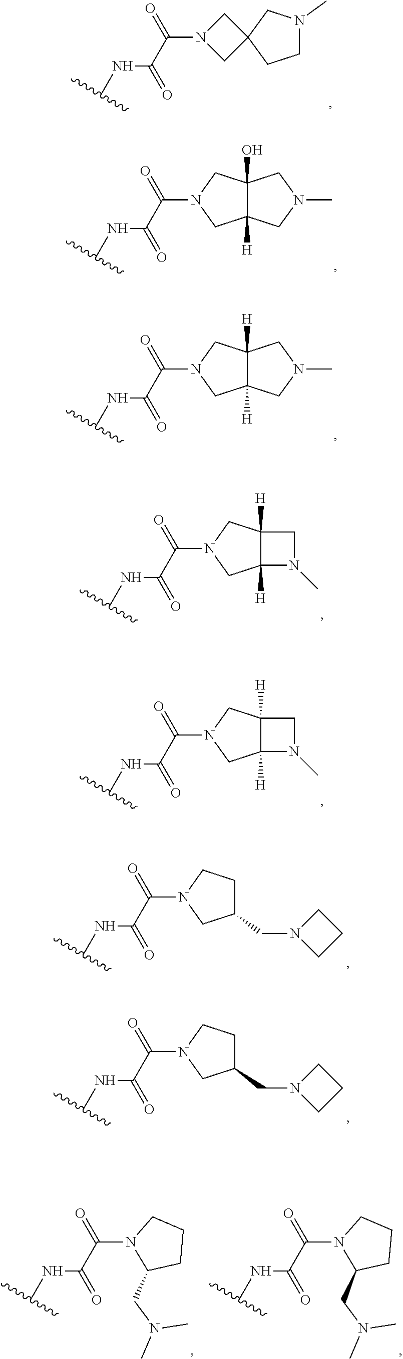 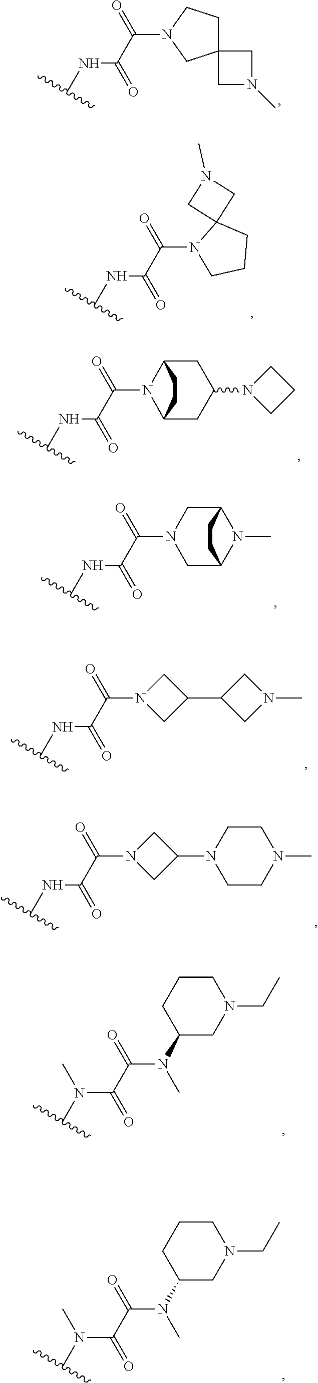 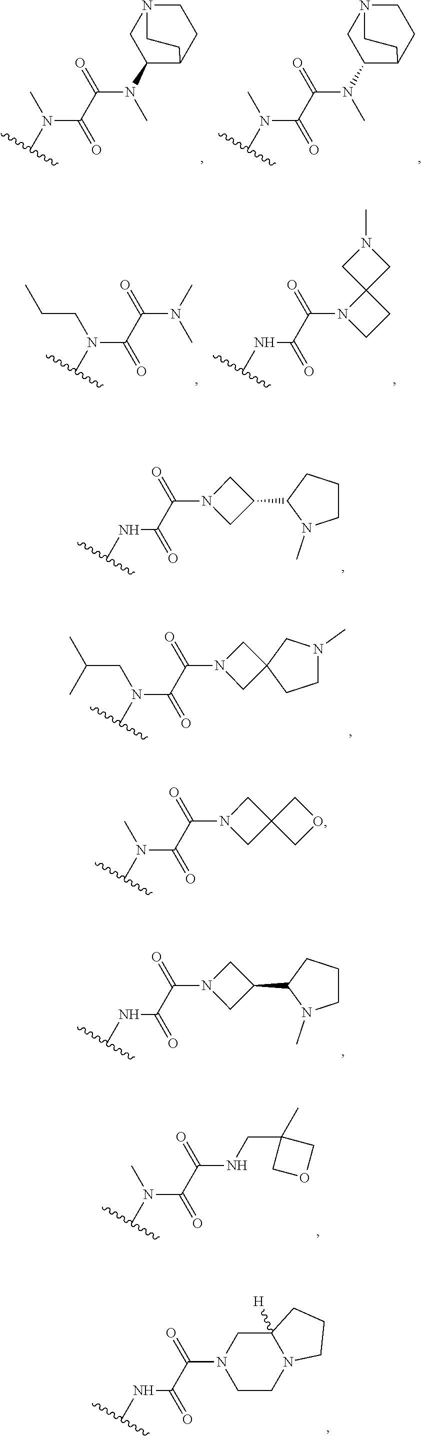 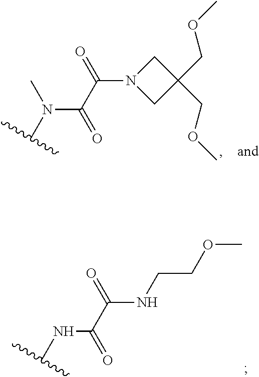 [SMILES: *N(C)C(=O)C(=O)N1CC(COC)(COC)C1 ; *NC(=O)C(=O)NCCOC]
or a pharmaceutically acceptable salt, single stereoisomer, mixture of stereoisomers, tautomer, or prodrug thereof.

2. The compound of claim 1, wherein R₁ is selected from:

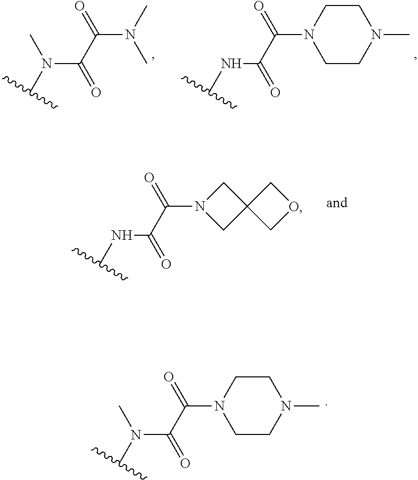

3. A compound of structural formula (I) 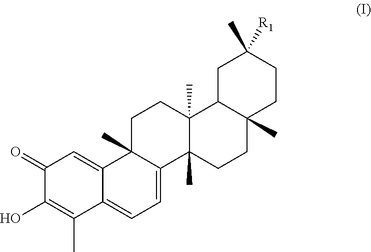
wherein,
R₁ is —NH—(CO)₂—NR₂R₃, or —N(CH₃)—(CO)₂—NR₂R₃;
R₂ and R₃ are independently selected from a hydrogen, an alkyl, a cycloalkyl, an alkoxy, a heterocycloalkyl, an alkylaryl, an alkenyl, an alkynyl, an aryl, an amine, or a heteroaryl, wherein each of the alkyl, cycloalkyl, alkoxy, heterocycloalkyl, alkylaryl, alkenyl, alkynyl, aryl, amine, or heteroaryl is optionally substituted with substituents individually selected from alkyl, alkoxy, cycloalkyl, ether, amine, optionally substituted with one or more alkyl, halogen, hydroxyl, cyano, nitrile, CF₃, ester, amide, cycloalkyl amide, sugar, heteroarylamide, optionally substituted with alkyl and/or alkoxy, urea, carbamate, thioether, sulfate, sulfonyl, sulfonic acid, carboxylic acid, and aryl; or
R₂ and R₃ taken together form a cycloalkyl, heterocycloalkyl, aryl, or heteraryl group, wherein each of the cycloalkyl, heterocycloalkyl, aryl, or heteraryl is optionally substituted with substituents individually selected from alkyl, cycloalkyl, alkoxy, heterocycloalkyl, alkylaryl, alkenyl, alkynyl, aryl, heteroaryl, amine, halogen, hydroxyl, ether, nitrile, cyano, nitro, CF₃, ester, amide, urea, carbamate, thioether, or carboxylic acid group;
or a pharmaceutically acceptable salt, single stereoisomer, mixture of stereoisomers, tautomer, or prodrug thereof.

4. The compound of claim 3, wherein R₁ is selected from:

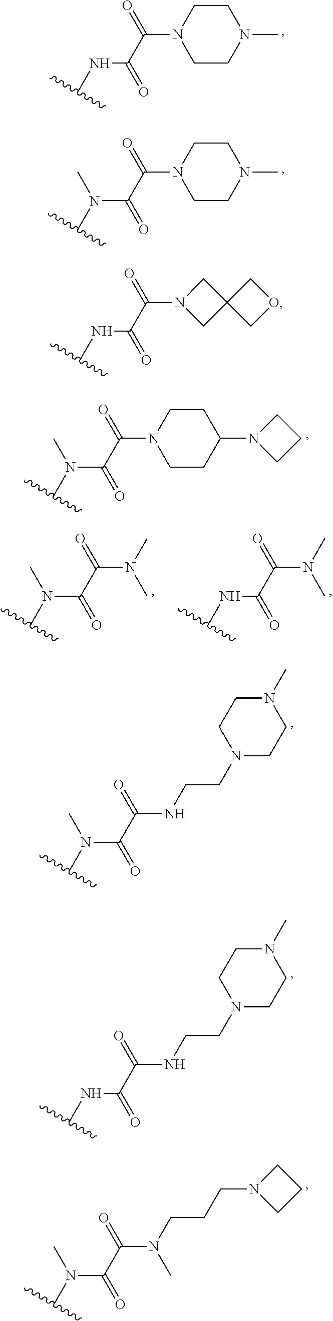

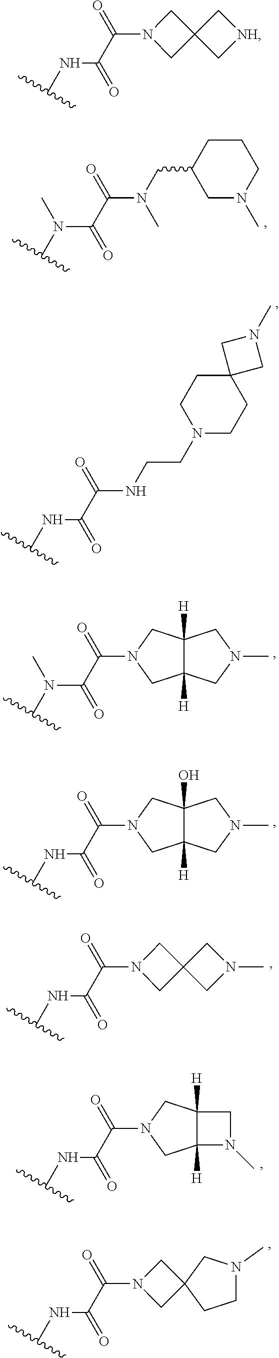

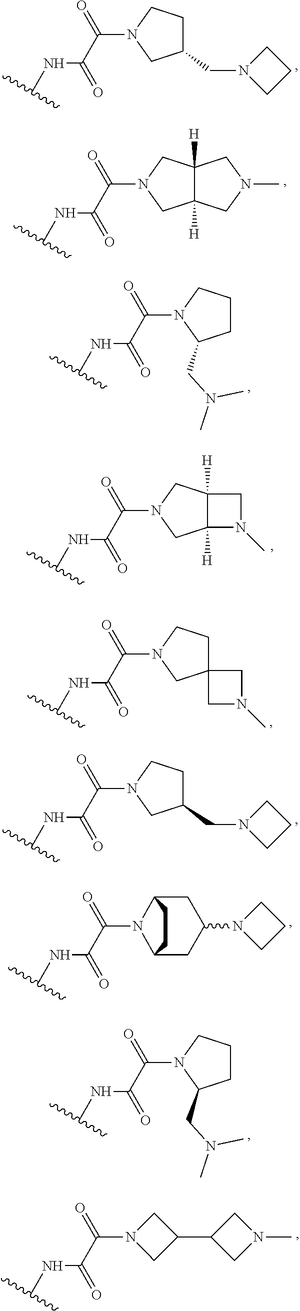

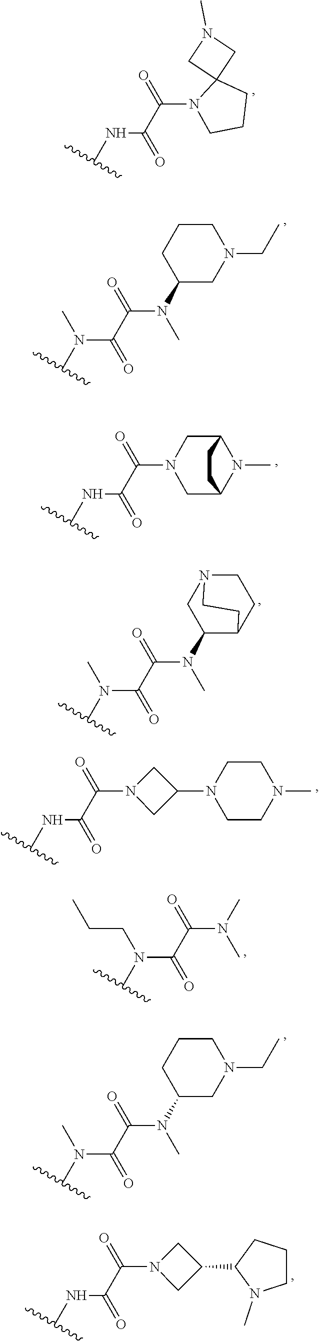

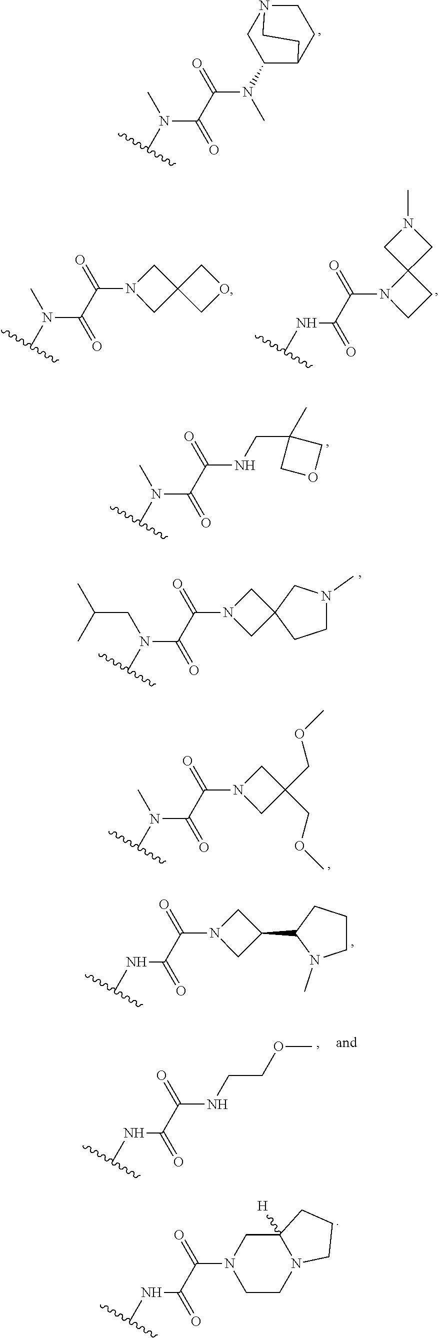

5. The compound of claim 1, wherein the compound is selected from compound 14, compound 15, compound 16, compound 17, compound 22, and compound 22-1 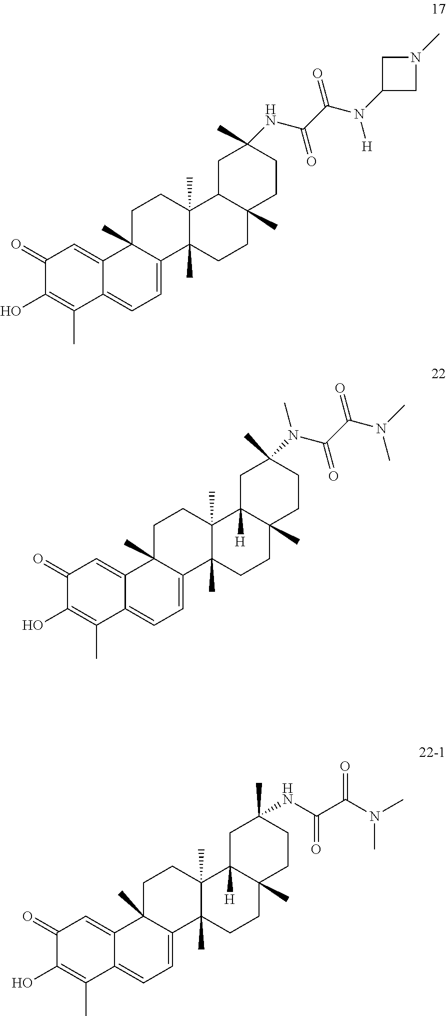
or a pharmaceutically acceptable salt, single stereoisomer, mixture of stereoisomers, tautomer, or prodrug thereof.

6. The compound of claim 1, wherein the compound is selected from compound 42, compound 54, compound 55, compound 56, compound 57, compound 58, compound 59, compound 60, compound 61, compound 62, compound 63, compound 64, compound 65, compound 66, compound 67, compound 68, compound 69, compound 70, compound 71, compound 72, compound 73, compound 74, compound 75, compound 76, compound 77, compound 78, compound 79, compound 80, compound 81, compound 82, compound 83, compound 84, compound 85, compound 86, compound 87, compound 88, compound 89, compound 90, compound 91, and compound 92 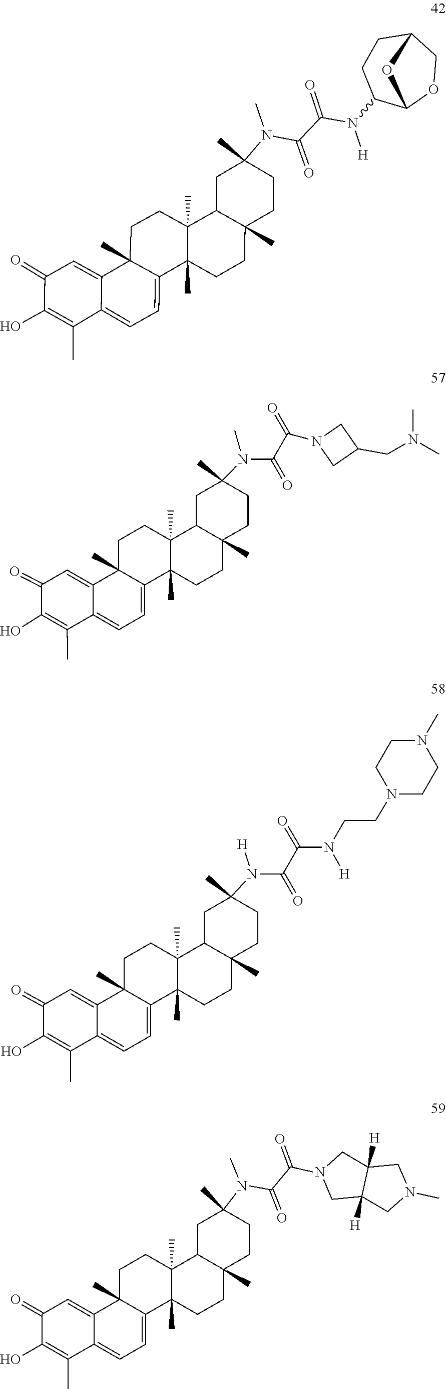 [SMILES: CC1=C(O)C(=O)C=C2C1=CC=C1[C@@]2(C)CC[C@@]2(C)C3C[C@](C)(N(C)C(=O)C(=O)NC4CC[C@@H]5CO[C@H]4O5)CC[C@]3(C)CC[C@]12C ; CC1=C(O)C(=O)C=C2C1=CC=C1[C@@]2(C)CC[C@@]2(C)C3C[C@](C)(N(C)C(=O)C(=O)N4CC(CN(C)C)C4)CC[C@]3(C)CC[C@]12C ; CC1=C(O)C(=O)C=C2C1=CC=C1[C@@]2(C)CC[C@@]2(C)C3C[C@](C)(NC(=O)C(=O)NCCN4CCN(C)CC4)CC[C@]3(C)CC[C@]12C ; CC1=C(O)C(=O)C=C2C1=CC=C1[C@@]2(C)CC[C@@]2(C)C3C[C@](C)(N(C)C(=O)C(=O)N4C[C@H]5CN(C)C[C@H]5C4)CC[C@]3(C)CC[C@]12C] 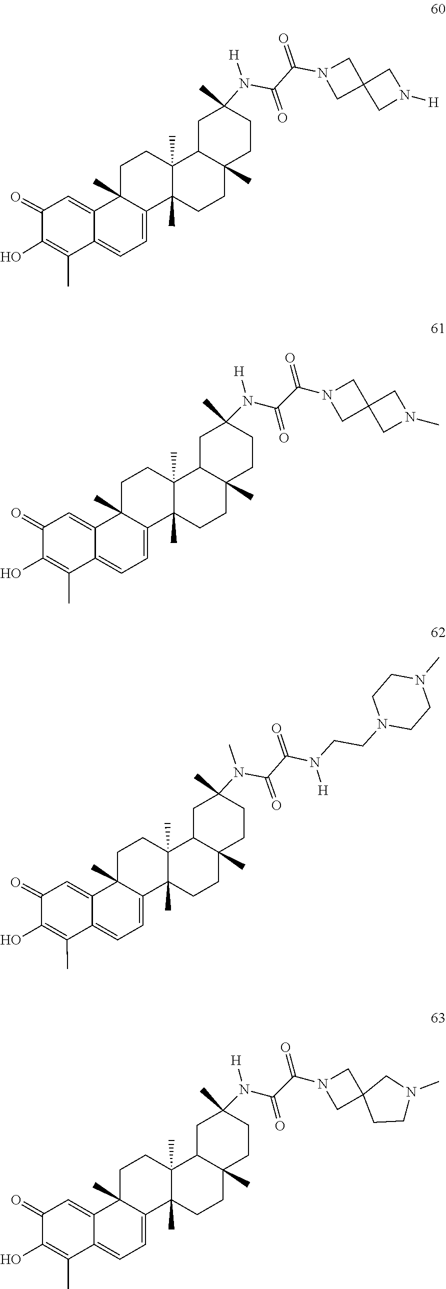 [SMILES: CC1=C(O)C(=O)C=C2C1=CC=C1[C@@]2(C)CC[C@@]2(C)C3C[C@](C)(NC(=O)C(=O)N4CC5(CNC5)C4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4CC5(CN(C)C5)C4)CC[C@]3(C)CC[C@]12C ; CC1=C(O)C(=O)C=C2C1=CC=C1[C@@]2(C)CC[C@@]2(C)C3C[C@](C)(N(C)C(=O)C(=O)NCCN4CCN(C)CC4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4CC5(CCN(C)C5)C4)CC[C@]3(C)CC[C@]12C] 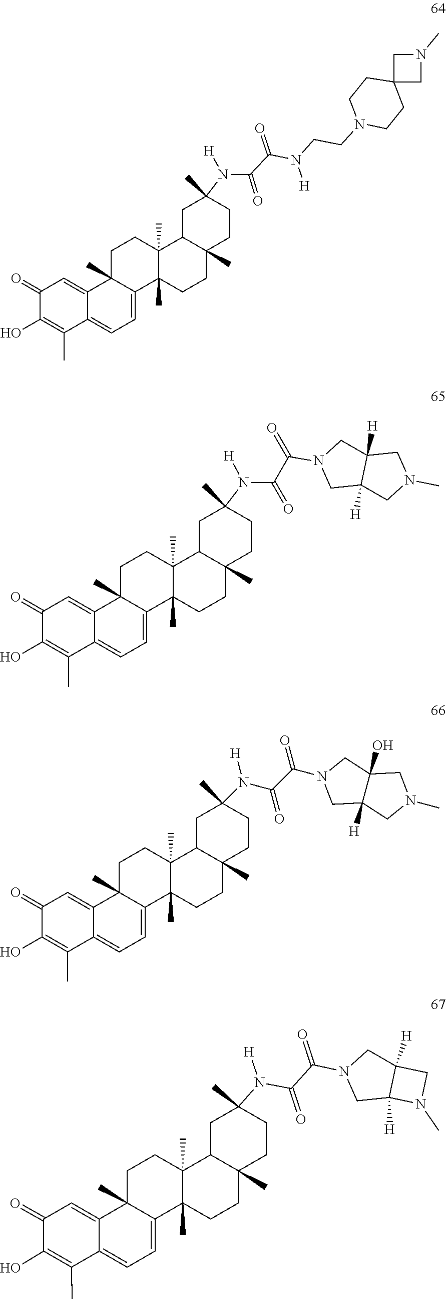 [SMILES: CC1=C(O)C(=O)C=C2C1=CC=C1[C@@]2(C)CC[C@@]2(C)C3C[C@](C)(NC(=O)C(=O)NCCN4CCC5(CC4)CN(C)C5)CC[C@]3(C)CC[C@]12C ; CC1=C(O)C(=O)C=C2C1=CC=C1[C@@]2(C)CC[C@@]2(C)C3C[C@](C)(NC(=O)C(=O)N4C[C@@H]5CN(C)C[C@H]5C4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4C[C@H]5CN(C)C[C@@]5(O)C4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4C[C@H]5CN(C)[C@H]5C4)CC[C@]3(C)CC[C@]12C] 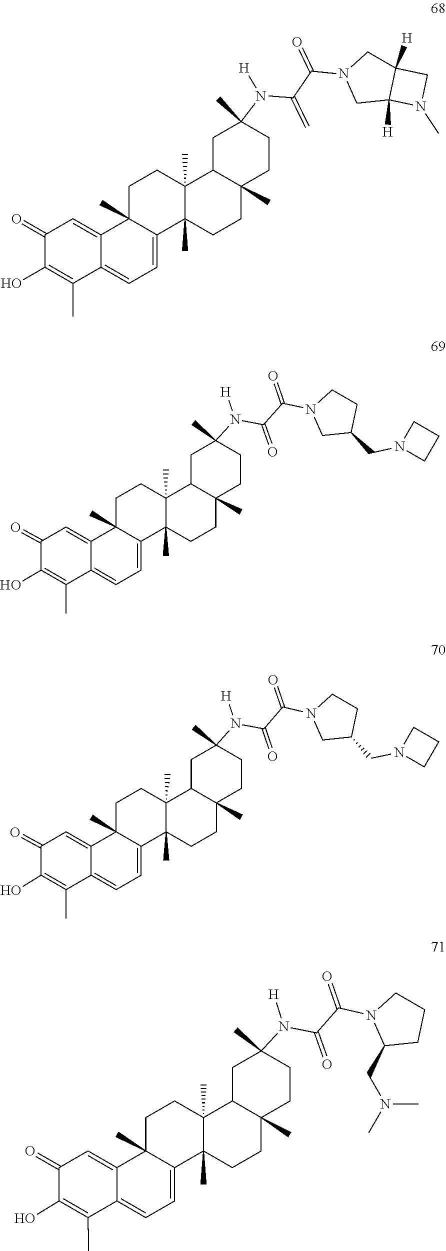 [SMILES: C=C(N[C@]1(C)CC[C@]2(C)CC[C@]3(C)C4=CC=C5C(=CC(=O)C(O)=C5C)[C@]4(C)CC[C@@]3(C)C2C1)C(=O)N1C[C@@H]2CN(C)[C@@H]2C1 ; CC1=C(O)C(=O)C=C2C1=CC=C1[C@@]2(C)CC[C@@]2(C)C3C[C@](C)(NC(=O)C(=O)N4CC[C@@H](CN5CCC5)C4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4CC[C@H](CN5CCC5)C4)CC[C@]3(C)CC[C@]12C ; CC1=C(O)C(=O)C=C2C1=CC=C1[C@@]2(C)CC[C@@]2(C)C3C[C@](C)(NC(=O)C(=O)N4CCC[C@H]4CN(C)C)CC[C@]3(C)CC[C@]12C] 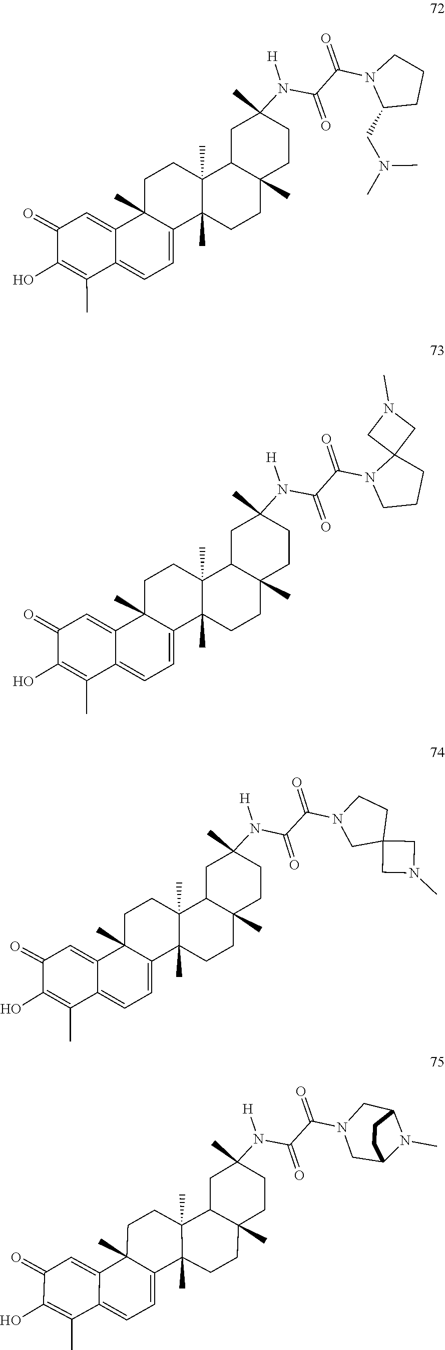 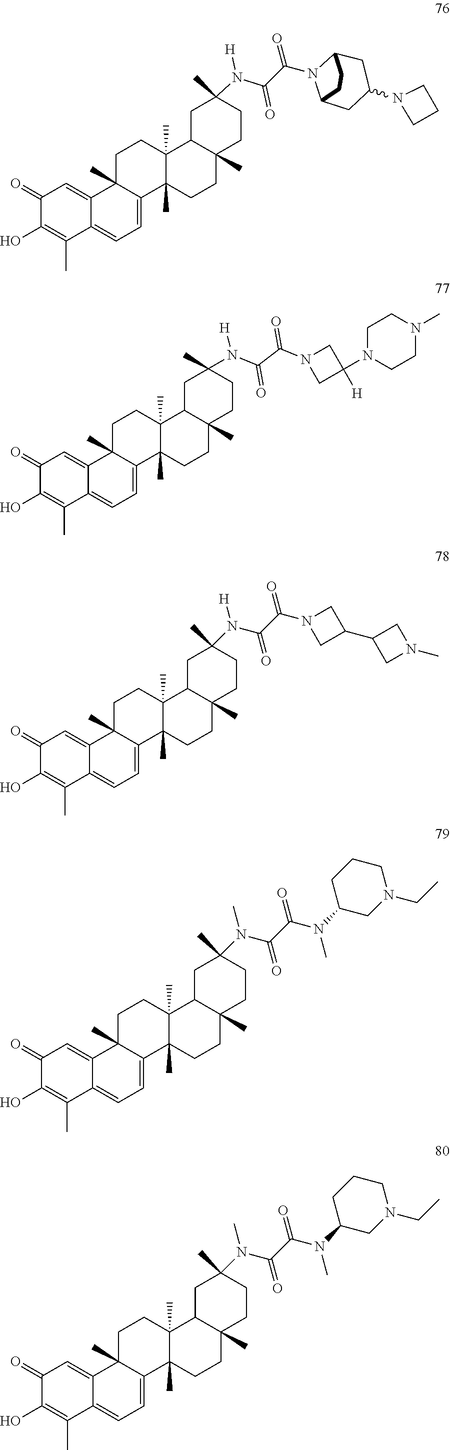 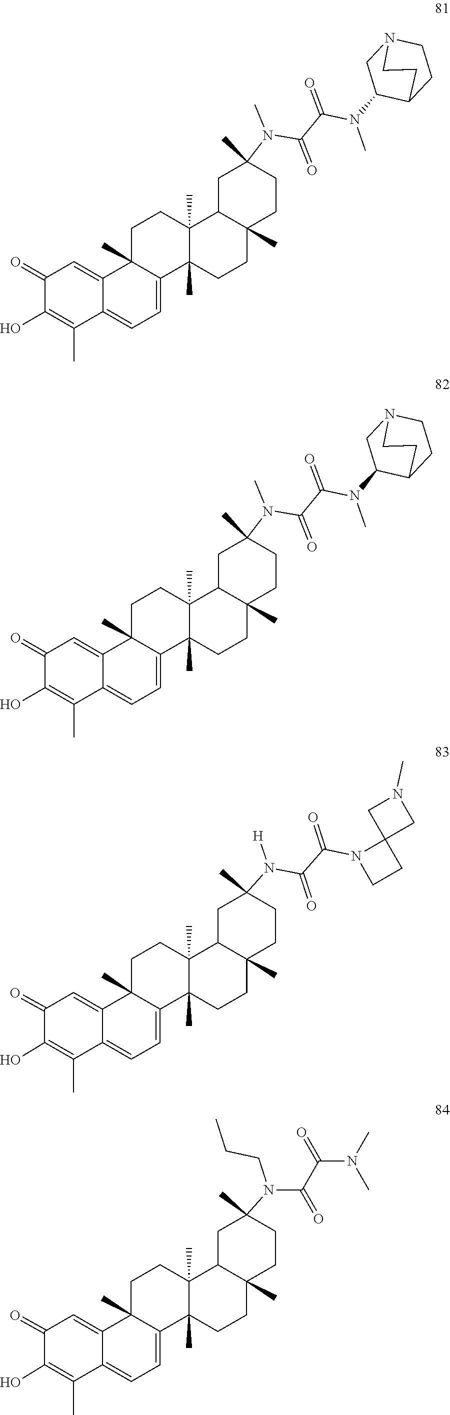 [SMILES: CC1=C(O)C(=O)C=C2C1=CC=C1[C@@]2(C)CC[C@@]2(C)C3C[C@](C)(N(C)C(=O)C(=O)N(C)[C@@H]4CN5CCC4CC5)CC[C@]3(C)CC[C@]12C ; CC1=C(O)C(=O)C=C2C1=CC=C1[C@@]2(C)CC[C@@]2(C)C3C[C@](C)(N(C)C(=O)C(=O)N(C)[C@H]4CN5CCC4CC5)CC[C@]3(C)CC[C@]12C ; CC1=C(O)C(=O)C=C2C1=CC=C1[C@@]2(C)CC[C@@]2(C)C3C[C@](C)(NC(=O)C(=O)N4CCC45CN(C)C5)CC[C@]3(C)CC[C@]12C ; CCCN(C(=O)C(=O)N(C)C)[C@]1(C)CC[C@]2(C)CC[C@]3(C)C4=CC=C5C(=CC(=O)C(O)=C5C)[C@]4(C)CC[C@@]3(C)C2C1] 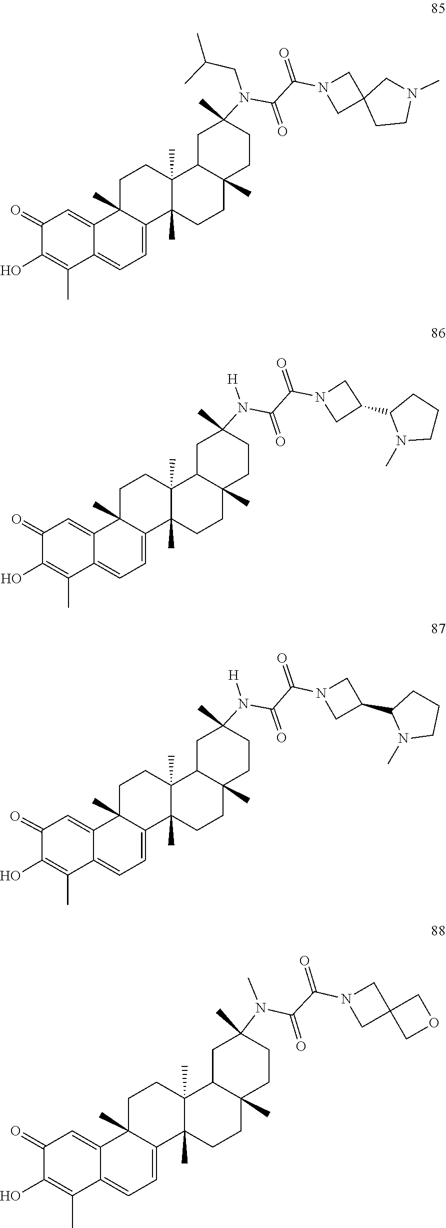 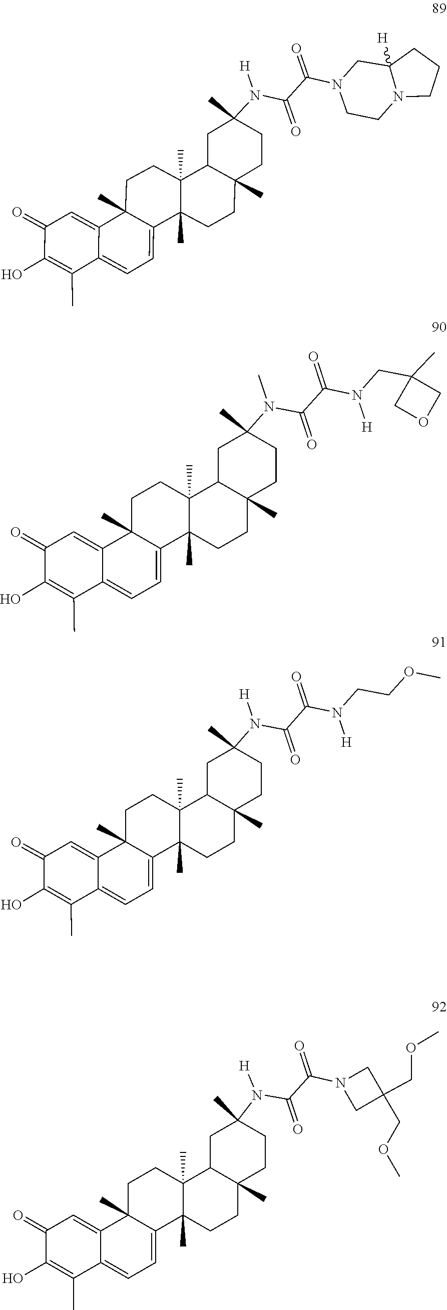 [SMILES: CC1=C(O)C(=O)C=C2C1=CC=C1[C@@]2(C)CC[C@@]2(C)C3C[C@](C)(NC(=O)C(=O)N4CCN5CCCC5C4)CC[C@]3(C)CC[C@]12C ; CC1=C(O)C(=O)C=C2C1=CC=C1[C@@]2(C)CC[C@@]2(C)C3C[C@](C)(N(C)C(=O)C(=O)NCC4(C)COC4)CC[C@]3(C)CC[C@]12C ; COCCNC(=O)C(=O)N[C@]1(C)CC[C@]2(C)CC[C@]3(C)C4=CC=C5C(=CC(=O)C(O)=C5C)[C@]4(C)CC[C@@]3(C)C2C1 ; COCC1(COC)CN(C(=O)C(=O)N[C@]2(C)CC[C@]3(C)CC[C@]4(C)C5=CC=C6C(=CC(=O)C(O)=C6C)[C@]5(C)CC[C@@]4(C)C3C2)C1]
or a pharmaceutically acceptable salt, single stereoisomer, mixture of stereoisomers, tautomer, or prodrug thereof.

7. The compound of claim 1, wherein the compound is characterized by having an IC₅₀ in a NF-kB reporter assay of about 1200 nM or less, about 1000 nM or less, 750 nM or less, 500 nM or less, or 300 nM or less.

8. A pharmaceutical composition comprising a compound of claim 1, and a pharmaceutically acceptable excipient.

9. A method of treating a celastrol-responsive disease or disorder in a subject suffering therefrom comprising administering to the subject a therapeutically effective amount of a compound of claim 1.

10. The method of claim 9, wherein the celastrol-responsive disease or disorder is a cancer; optionally, wherein the cancer is selected from: gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme (GBM).

11. The method of claim 9, wherein the celastrol-responsive disease or disorder is an inflammatory and/or autoimmune disorder; optionally, wherein the inflammatory and/or autoimmune disorder is selected from rheumatoid arthritis (RA), multiple sclerosis (MS), ankylosing spondylitis, systemic lupus erythematosus (SLE), inflammatory bowel disease, osteoarthritis (OA), acute respiratory distress syndrome (ARDS), Guillain-Barré syndrome (GBS), Sickle cell disease (SCD), allergy (e.g. Asthma), psoriasis and other inflammatory skin conditions.

12. The method of claim 9, wherein the celastrol-responsive disease or disorder is a neurological disorder; optionally, wherein the neurological disorder is selected from Parkinson's disease, Huntington disease, Alzheimer's disease, and amyotrophic lateral sclerosis (ALS), and Gaucher disease (GD).

13. The method of claim 9, wherein the celastrol-responsive disease or disorder is an obesity-related disease or disorder; optionally, wherein the obesity-related disease or disorder is selected from obesity, pre-obesity, morbid obesity, type 2 diabetes (T2D), atherosclerosis, diabetic nephropathy, gout, cardiac fibrosis, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome, and MOMO (Macrosomia Obesity Macrocephaly Ocular abnormalities) Syndrome.

14. The method of claim 9, wherein the celastrol-responsive disease or disorder is a liver-related disease or disorder; optionally, wherein the liver-related disease or disorder is selected from acute-chronic liver failure (ACLF), alcoholic liver disease, cholestatic liver disease, drug-induced liver disease, hepatocellular carcinoma, non-alcoholic fatty liver disease (NAFLD), non-alcoholic steatohepatitis (NASH), viral hepatitis, and viral liver disease.

15. The method of claim 9, wherein the celastrol-responsive disease or disorder is a brain injury or brain disorder; optionally, wherein the brain-related injury or disorder is selected from middle cerebral artery occlusion (MCAO)-induced brain injury, cerebral ischemia/reperfusion (I/R) injury, Vascular dementia (VD), and acute ischemic stroke-induced brain injury.

16. The method of claim 9, wherein the celastrol-responsive disease or disorder is an ocular disorder or injury; optionally, wherein the ocular-related injury or disorder is selected from dry eye disease, ocular inflammation, age-related macular degeneration (AMD), subconjunctival fibrosis, ocular hypertension-induced degeneration of the retina, bright light-induced degeneration, macrophage-induced corneal neovascularization, corneal allograft survival, and optic nerve crush.

17. The method of claim 9, wherein the compound of pharmaceutical composition is administered in combination with another therapy.

18. The method of claim 9, wherein administering comprises oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

19. The method of claim 9, wherein the compound or pharmaceutical composition is administered in a form selected from the group comprising pills, capsules, tablets, granules, powders, salts, crystals, liquid, serums, syrups, suspensions, gels, creams, pastes, films, patches, and vapors.

20. The method of claim 9, wherein is the subject is a mammal.

21. The method of claim 9, wherein the subject is a human.

22-25. (canceled)
